(19) United States
(12) Patent Application Publication
(10) Pub. No.: US 2025/0256085 A1
(43) Pub. Date: Aug. 14, 2025

(54) SYSTEMS AND METHODS FOR A DESCENDING AORTA PERISTALISIS HEART ASSIST PUMP

(71) Applicant: PHAP MEDICAL, LLC, Trabuco Canyon, CA (US)

(72) Inventors: Michael Saveliev, Trabuco Canyon, CA (US); Steve Smith, Trabuco Canyon, CA (US); Brian Babson, Trabuco Canyon, CA (US); Babek Kahmenian, Trabuco Canyon, CA (US)

(21) Appl. No.: 19/196,584

(22) Filed: May 1, 2025

Related U.S. Application Data
(63) Continuation-in-part of application No. 18/671,350, filed on May 22, 2024.
(60) Provisional application No. 63/641,266, filed on May 1, 2024.
(60) Provisional application No. 63/503,561, filed on May 22, 2023.

(51) Int. Cl. A61M 60/139; A61M 60/157; A61M 60/295; A61M 60/34; A61M 60/468; A61M 60/497; A61M 60/515; A61M 60/843

(52) U.S. Cl. CPC A61M 60/139; A61M 60/157; A61M 60/295; A61M 60/34; A61M 60/468; A61M 60/497; A61M 60/515; A61M 60/843; A61M 2205/3331; A61M 2205/50; A61M 2210/127

(57) ABSTRACT

A bidirectional intravascular blood pump system is disclosed for deployment in the descending aorta to improve perfusion in both upper and lower extremities. In some embodiments, the system includes a flexible stent housing containing an inflatable pump chamber and selectively inflatable proximal and distal check valves. In some embodiments, a controller sequences inflation and deflation of the pump components in coordination with the cardiac cycle. In some embodiments, the system capable of dynamically providing flow in opposite directions within the descending aorta to optimize systemic and cerebral perfusion. In some embodiments, the system can direct blood flow in either direction through the aorta by an inflation sequence of the check valves and pump chamber. In some embodiments, one or more bypasses in a check element, stent, and/or formed from selective inflation, enable pressurization of the aortic arch when timed with the closing of the aortic valve.

### CROSS-REFERENCE TO RELATED APPLICATION

This application is a continuation in part of non-provisional application Ser. No. 18/671,350, filed May 22, 2024, which claims the benefit of and priority to U.S. Provisional Patent Application No. 63/503,561, filed May 22, 2023, the entire content of each of which is incorporated by reference herein. This application is also a non-provisional application which claims the benefit of and priority to U.S. Provisional Patent Application No. 63/641,266, filed May 1, 2024, the entire content of each of which is incorporated by reference herein.

### BACKGROUND

Intravascular circulatory support devices such as intra-aortic balloon pumps (IABPs) and impeller-based systems have been used for supporting patients with acute cardiac conditions. However, these unidirectional pumps primarily assist either left ventricular unloading or augment downstream perfusion without regard for balanced systemic distribution.

In particular, balloon-based pumps inflate during diastole to push blood downstream, but their directional rigidity and inability to alternate flow reduce their efficacy in certain aortic pathologies and in supporting upstream territories. Moreover, fixed flow direction may contribute to perfusion imbalance, potentially under-perfusing critical organs.

Therefore, there is a need for a pump system capable of dynamically alternating flow direction within the descending aorta, as well as control system to synchronize with the cardiac cycle, to address such limitations and to optimize systemic and cerebral perfusion.

### SUMMARY

In some embodiments, the present disclosure is directed to a pump configured for intravascular placement and blood propulsion within a descending aorta. In some embodiments, the pump includes a stent, a pumping chamber located within the stent, one or more pump elements positioned within the pumping chamber, and at least a first check element configured to regulate fluid flow through a chamber end. In some embodiments, the pumping chamber includes a chamber first end and a chamber second end, and the one or more pump elements are configured to force fluid through both the chamber first end and the chamber second end simultaneously, thereby enabling bidirectional perfusion.

In some embodiments, the first check element is configured to prevent backflow through the chamber first end, and in some embodiments, a second check element is provided and configured to regulate flow through the chamber second end. In some embodiments, one or more of the check elements include passive valve structures, such as multi-leaf check valves, or active elements such as inflatable balloon check valves. In some embodiments, one or more check elements include bypass features, such as one or more flow channels or orifices configured to permit a controlled degree of backflow for the purpose of pressurizing the aortic arch or perfusing upper extremities.

In some embodiments, the pump includes a controller configured to cause the one or more pump elements to inflate in synchronization with a cardiac cycle, for example when the aortic valve is closed, enabling coordinated augmentation of native heart function. In some embodiments, the one or more pump elements include a first pump element configured to direct fluid through the chamber first end and a second pump element configured to direct fluid through the chamber second end. In some embodiments, an inflatable chamber separator is positioned between the first pump element and the second pump element, and/or is configured to isolate the respective chamber regions during pump operation.

In some embodiments, the pump further includes one or more inflatable stent shields positioned circumferentially around an outer surface of the stent. In some embodiments, the one or more inflatable stent shields are configured to contact a wall of an aorta and provide a soft interface to reduce abrasion and promote anchoring. In some embodiments, the one or more inflatable stent shields include flow channels, such as longitudinal grooves, and/or are formed as a plurality of spaced inflatable segments configured to allow bypass flow between the stent and the aortic wall, thereby promoting branch perfusion and minimizing stagnation.

In some embodiments, the pump is configured for percutaneous delivery through a catheter having a diameter between 10 to 12 French. In some embodiments, one or more portions of the stent include a first tapered region and a second tapered region configured to house the first check element and second check element, respectively. In some embodiments, the stent, pump elements, and check elements are configured to be compacted for delivery and self-expand or otherwise deploy upon release from a catheter sheath.

These and other embodiments disclosed herein enable bidirectional, regulated blood flow through the descending aorta while preserving branch perfusion, synchronizing with the cardiac cycle, and minimizing vascular trauma.

### DRAWINGS DESCRIPTION

FIG. 1 illustrates a system overview, including graphical user interfaces, controllers, element fluid pumps, pistons, heart assist pumps, and stents, according to some embodiments.

FIG. 2 shows a non-limiting example of a graphical user interface (GUI) configured to display system parameters and accept user inputs, according to some embodiments.

FIG. 3 depicts an element filling operation executed by the system in a tubular element arrangement, showing stages of inflation and deflation, according to some embodiments.

FIG. 4 illustrates a heart assist pump with a stent arrangement comprising one or more elements and stents, according to some embodiments.

FIG. 5 shows the operation of the stent arrangement, according to some embodiments.

FIG. 6 depicts a first filling step and a first deflating step in a balloon cycle, according to some embodiments.

FIG. 7 illustrates a second filling step and a second deflating step, according to some embodiments.

FIG. 8 shows a third filling step and a third deflating step, according to some embodiments.

FIG. 9 is a continuation of the inflation and deflation illustrations of FIG. 8, according to some embodiments.

FIG. 10 is a continuation of the inflation and deflation illustrations of FIG. 9, according to some embodiments.

FIG. 11 depicts a final inflation step and a final deflation step, according to some embodiments.

FIGS. 12 and 13 show non-limiting renderings of a single element configuration, according to some embodiments.

FIG. 14 illustrates a plural balloon system comprising two elements, showing their sequential inflation and deflation, according to some embodiments.

FIG. 15 depicts a three-balloon arrangement, according to some embodiments.

FIG. 16 shows a sequential element arrangement comprising a plurality of elements, according to some embodiments.

FIG. 17 illustrates a tapered-shaped element, according to some embodiments.

FIG. 18 depicts the collapse of the cone-shaped pocket during inflation, according to some embodiments.

FIG. 19 shows one or more check elements, including multi-leaf configurations and their interaction with inflating balloons, according to some embodiments.

FIG. 20 illustrates a heart assist pump with a mandrel flow chamber, according to some embodiments.

FIG. 21 depicts a multi-element fluid pump arrangement, according to some embodiments.

FIG. 22 illustrates steps implemented by the system, including inflation and deflation sequences, manual and automatic modes, and sensor-based controls, according to some embodiments.

FIG. 23 shows a computer system enabling or comprising the systems and methods, including processors, interfaces, and software modules, according to some embodiments.

FIG. 24 depicts a pleated element arrangement in a partially inflated configuration, according to some embodiments.

FIG. 25 illustrates the directional inflation of a pleated element, according to some embodiments.

FIG. 26 shows a non-limiting example of an inlet check element in an inflated configuration, according to some embodiments.

FIG. 27 depicts side and front views of a check element and a directional pumping element, according to some embodiments.

FIG. 28 illustrates an inflated bi-directional pumping element, according to some embodiments.

FIG. 29 shows an hourglass configuration for the heart assist pump, according to some embodiments.

FIG. 30 depicts non-limiting example dimensions for the LV pumping element, aorta pumping element, and aorta check valve, according to some embodiments.

FIG. 31 illustrates non-limiting example dimensions for the stent and mandrel, including ranges for major diameters, according to some embodiments.

FIG. 32 shows a deployment configuration of the heart assist pump, according to some embodiments.

FIG. 33 depicts a flow chamber filling cycle, according to some embodiments.

FIG. 34 illustrates a flow chamber sealing cycle, according to some embodiments.

FIG. 35 shows a left ventricle flow chamber pump cycle, according to some embodiments.

FIG. 36 depicts an aortic flow chamber, according to some embodiments.

FIG. 37 illustrates an aortic seal and pump cycle, according to some embodiments.

FIG. 38 shows a completed pump cycle, according to some embodiments.

FIG. 39 depicts another configuration of the heart assist pump with an additional check valve, according to some embodiments.

FIG. 40 illustrates placement of pump sections in the ascending aorta and left ventricle, according to some embodiments.

FIG. 41 shows a first rendered sectional view of an installed system, according to some embodiments.

FIG. 42 depicts a second rendered sectional view of the installed system, according to some embodiments.

FIG. 43 illustrates a third rendered sectional view of the installed system, according to some embodiments.

FIG. 44 shows a fourth rendered sectional view of the installed system, according to some embodiments.

FIG. 45 depicts a fifth rendered sectional view of the installed system, according to some embodiments.

FIG. 46 illustrates a sixth rendered sectional view of the installed system, according to some embodiments.

FIG. 47 shows the system deployed as a descending aorta pump, according to some embodiments.

FIG. 48 depicts an inflation and deflation sequence for intra-aortic pump actuation, according to some embodiments.

FIG. 49 illustrates a descending aorta pump for bidirectional blood flow control, according to some embodiments.

FIG. 50 depicts a descending aorta pump, according to some embodiments.

FIG. 51 illustrates a descending aorta pump, according to some embodiments.

FIG. 52 shows a descending aorta pump, according to some embodiments.

FIG. 53 depicts a descending aorta pump, according to some embodiments.

FIG. 54 illustrates a descending aorta pump, according to some embodiments.

FIG. 55 shows a descending aorta pump shield configuration, including inflatable stent shields for vascular protection, according to some embodiments.

FIG. 56 illustrates an end view of FIG. 55, according to some embodiments.

FIG. 57 depicts a directional flow control configuration, according to some embodiments.

FIG. 58 shows various features of a non-limiting stent arrangement, according to some embodiments.

### DETAILED DESCRIPTION

In some embodiments, the disclosure is directed to a system for pumping blood that is configured to minimize or lessen damage to blood cells. In some embodiments, the system is configured for deployment in various anatomical positions, including fully within the descending aorta. In some embodiments, the system comprises one or more element fluid pumps, one or more element tubes, one or more elements, and/or one or more stents. In some embodiments, the system is shaped or contoured to conform to the interior wall of the aorta and is configured to generate bidirectional flow to support perfusion in multiple vascular directions. In some embodiments, the one or more elements are housed within the stent. In some embodiments, the one or more elements are configured to receive a fluid (e.g., gas) from the one or more element tubes. In some embodiments, the one or more element pumps are configured to execute an inflation and/or a deflation of the one or more elements via the one or more element tubes.

In some embodiments, the system includes one or more element housings. In some embodiments, the one or more elements are housed in the one or more element housings. In some embodiments, the one or more element housings include the stent. In some embodiments, the one or more element housings include a tube and/or catheter. In some embodiments, the one or more elements are configured to pump a liquid through the one or more element housings as a result of the inflation and/or the deflation of the one or more elements. In some embodiments, the system is configured to operate with directional inflation in either or both axial directions. In some embodiments, the directional inflation is configured to force a liquid (e.g., blood) in one direction, including toward the aortic arch when deployed in the descending aorta.

In some embodiments, each of the one or more elements comprises an element inlet end and an outlet end. In some embodiments, each of the one or more elements is configured to enable an element inlet end to expand before an outlet end expands. In some embodiments, this configuration promotes efficient unidirectional flow, even when positioned away from the heart.

In some embodiments, at least one of the one or more elements includes a tubular shape. In some embodiments, the tubular shape includes an element inlet end, an element outlet end, and an element hollow center portion. In some embodiments, the tubular shape is configured to enable a liquid to flow around the element, preventing damage to the blood vessels. In some embodiments, the at least one element is configured to maintain a compact device profile during insertion.

In some embodiments, the system further includes a mandrel. In some embodiments, the mandrel is positioned within a center portion of the one or more element housings in the stent. In some embodiments, the one or more elements are coupled to the mandrel. In some embodiments, the one or more elements are configured to execute an inflation to cause the elements to expand from the mandrel toward the one or more element housings. In some embodiments, the expansion is configured to cause liquid to be pumped out of the housing in a single direction. In some embodiments, when used in descending aorta applications, the system is configured to deliver fluid in the proximal direction toward the aortic arch, where the controller is configured to time the fluid delivery while the aortic valve is closed.

In some embodiments, a tube and/or a mandrel is configured to support both positive pressure and vacuum ranges in the same structure. In some embodiments, the mandrel includes one or more upstream pressure sensors and/or one or more downstream pressure sensors. In some embodiments, the one or more pressure sensors are configured to sense liquid (e.g., blood) pressure. In some embodiments, references to a tube and/or mandrel are interchangeable when defining the metes and bounds of the system, and the term tube may also be used to refer to internal structures such as fill lumens that carry element fluid. In some embodiments, a tube and/or mandrel is configured to support gas volume consistent with cycle times by balancing inflation and deflation times. In some embodiments, a tube and/or a mandrel is configured to support a minimum of 500,000 cycles. In some embodiments, a tube and/or a mandrel includes a flexible material configured to be inserted through the vasculature and positioned without passing through the aortic valve. In some embodiments, a tube and/or a mandrel is configured to be abrasion resistant to a minimum of 500,000 cycles. In some embodiments, a tube and/or a mandrel is configured to be compatible with blood and other medical fluids added to a patient's blood. In some embodiments, a tube and/or a mandrel includes a Food and Drug Administration (FDA) compliant material.

In some embodiments, one or more elements are shaped, formed, and/or configured to directionally inflate. In some embodiments, one or more elements include a variable material thickness. In some embodiments, one or more elements include variable material durometers. In some embodiments, one or more elements include a material compatible with blood and/or medical fluids. In some embodiments, one or more elements include an FDA compliant material. In some embodiments, one or more elements include Pellethane® and/or similar materials. In some embodiments, one or more elements are abrasion resistant to a minimum of 600,000 cycles. In some embodiments, one or more elements are configured to inflate and deflate a minimum of 600,000 cycles. In some embodiments, the pump system includes element that project away from the outer surface of a stent, where the projecting elements are configured to engage with the inner wall of the descending aorta to form a shield, protecting the aortic wall and extending the operational life of the system.

In some embodiments, the one or more elements include a first element and a second element. In some embodiments, the first element comprises a first inflated volume. In some embodiments, the second element comprises a second inflated volume. In some embodiments, the first inflated volume is less than the second inflated volume. In some embodiments, the first element is positioned before the second element in the one or more housings relative to pumping direction. In some embodiments, inflation of the first element is configured to close a housing inlet end into the one or more housings. In some embodiments, inflation of the second element is configured to pump liquid from a first outlet end of the first element to a second outlet end of the second element.

In some embodiments, the system further comprises a third element. In some embodiments, the third element comprises a third inflated volume. In some embodiments, the third inflated volume is less than the second inflated volume. In some embodiments, the third element is positioned after the second element in the one or more housings relative to pumping direction. In some embodiments, inflation of the third element is configured to close a housing outlet end out of the one or more housings. In some embodiments, when deployed in a segment of the aorta, such as the descending thoracic or abdominal portion, the relative positioning of the elements is configured to generate a proximal-to-distal or distal-to-proximal flow path, depending on pump sequencing.

In some embodiments, the system further comprises one or more of a controller, a first element, and a second element. In some embodiments, the one or more elements include the first element and the second element. In some embodiments, the controller is configured to execute a first inflation of the first element before executing a second inflation of the second element. In some embodiments, the system includes a third element. In some embodiments, the one or more elements include the third element. In some embodiments, the controller is configured to execute a third inflation of the third element after executing the second inflation of the second element. In some embodiments, the inflation pattern and/or element fill percentage is selected to force flow against natural circulation pathways, for example, toward the aortic arch.

In some embodiments, the system includes a graphical user interface (GUI). In some embodiments, the GUI is configured to enable a user to execute a first pumping sequence configured to pump liquid in a first direction. In some embodiments, the GUI is configured to enable a user to execute a second pumping sequence configured to pump liquid in at least a second direction. In some embodiments, the controller is configured to execute a first deflation of the first element after executing a second deflation of the second element. In some embodiments, the controller is configured to execute a third deflation of the third element before executing a second deflation of the second element. In some embodiments, the controller is configured to execute an overlapping inflation of one or more elements. In some embodiments, the controller is configured to execute an overlapping deflation of one or more elements. In some embodiments, these configurations support bidirectional flow operation and are adapted for augmenting perfusion of target vascular regions including the coronary and upper body circulation.

In some embodiments, the first pumping element is configured to directionally inflate. In some embodiments, the first pumping element is configured to at least partially seal an inlet of the heart assist pump when partially inflated. In some embodiments, the second pumping element is configured to directionally inflate. In some embodiments, the second pumping element is configured to at least partially seal an outlet of the reduced cross-section when partially inflated.

In some embodiments, the heart assist pump further comprises one or more check elements. In some embodiments, a check element is configured to prevent fluid flow from an outlet of the heart assist pump into a second pump section. In some embodiments, the second pump section comprises the second pumping element. In some embodiments, the check element is inflatable. In some embodiments, the check element is not inflatable. In some embodiments, the check element is configured to prevent fluid flow from an inlet of the heart assist pump into a first pump section.

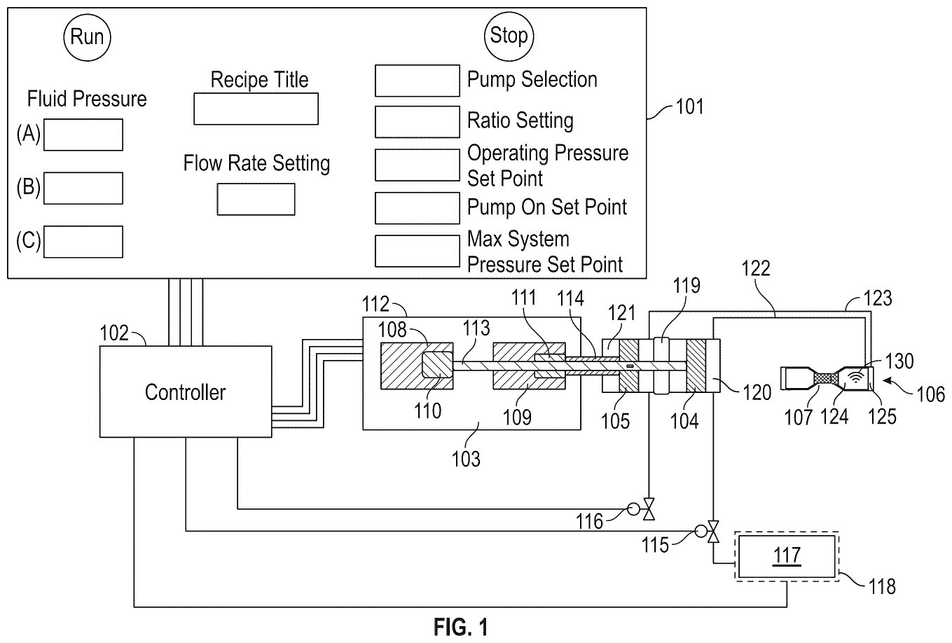

As shown in FIG. 1, in some embodiments, the peristalsis heart assist pump (PHAP) system (hereafter the “system”) includes one or more: graphical user interfaces 101 (GUIs), controllers 102, element fluid pumps 103, pistons 104, 105, heart assist pumps 106, and/or stents 107. As used herein, the term “element fluid” refers to the medium used to inflate and/or deflate a pumping element, which may be a liquid or a gas according to some embodiments. The term “liquid” refers to the medium that is being pumped by the heart assist pump 106, which includes blood as a non-limiting example according to some embodiments. In some embodiments, the heart assist pump 106 is configured to be used as a peristalsis heart assist pump. In some embodiments, the heart assist pump 106 comprises the one or more elements 124, 125.

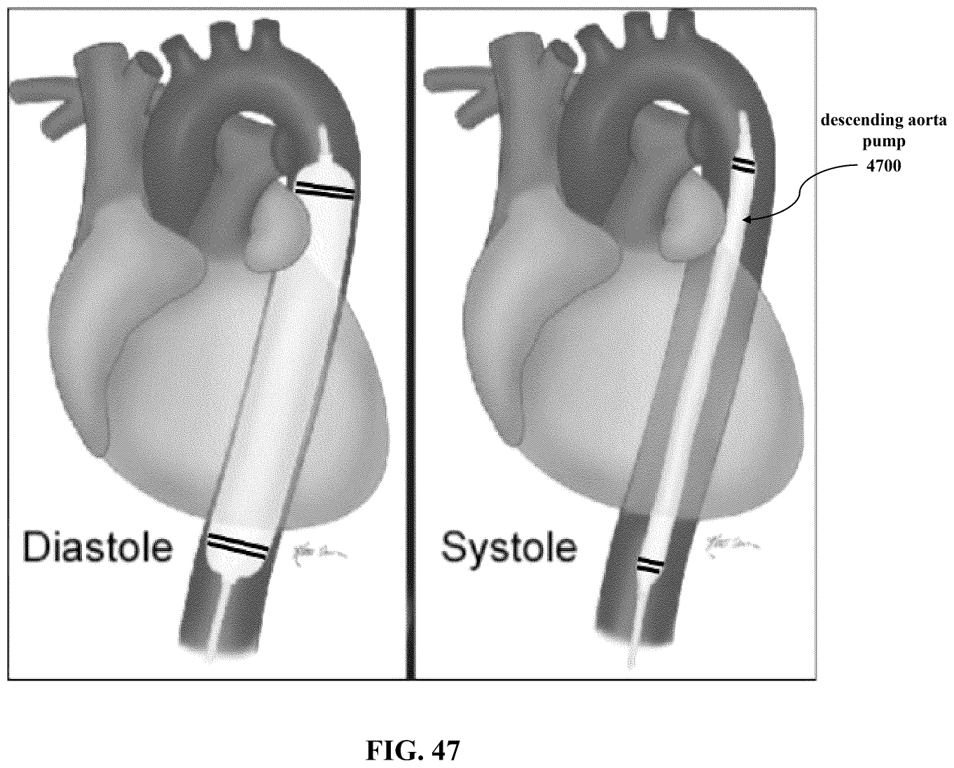

The system includes various configurations of structures and elements for the heart assist pump 106, such as those shown in FIGS. 47-58. While this detailed description of some embodiments of pump 106 are described herein, they are not taking to be limiting. For example, any of the elements shown in FIGS. 47-58 can be replaced with and/or be augmented with any element described in relation to any of the figures to achieve a desired function for a particular application, which may include areas outside the heart or in pumping applications that do not include a cardiovascular system.

In some embodiments, the one or more elements 124, 125 include one or more balloons 124, 125. As used herein, any reference to a balloon is also a reference to the broader genus (pumping) element, where the terms are interchangeable for the purposes of defining the metes and bounds of the system. As used herein, a balloon (element) includes any elastic material that is configured to expand when a fluid pressure is supplied and contract when the fluid pressure is removed. In some embodiments, the elastic material includes medical grade material suitable for insertion into the human body.

In some embodiments, one or more elements 124, 125 are configured to control the flow of liquid. In some embodiments, the one or more elements 124, 125 are inflatable and/or collapsible under pressure and/or vacuum, respectively. In some embodiments, non-limiting example elements include inflatable elements, collapsible elements, check elements, umbrella elements, flexible elements, and/or magnetic elements, where the modifier (e.g., inflatable) serves to describe the element's function and/or structure.

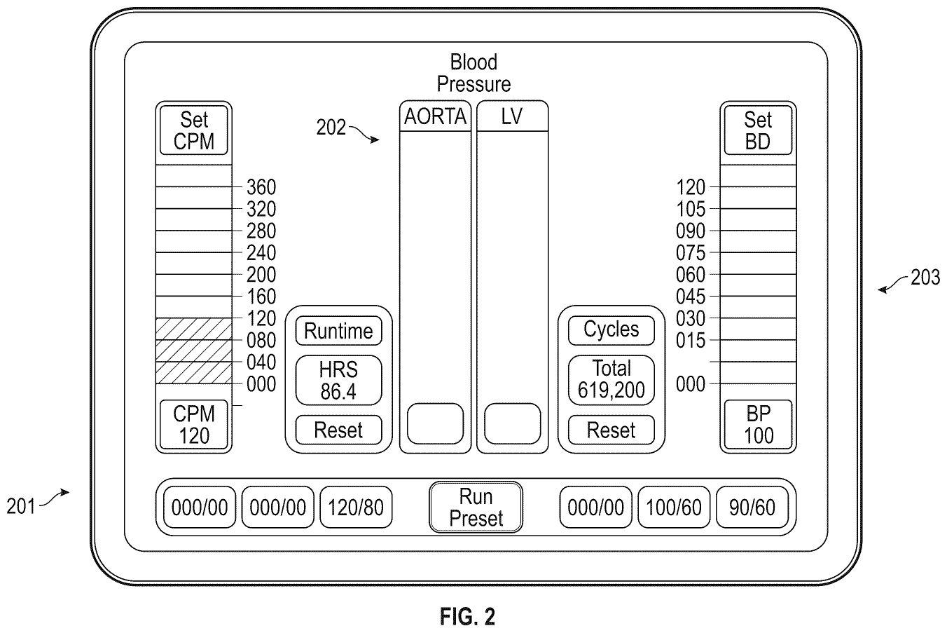

The system includes a graphical user interface 101 (GUI) configured to accept one or more user inputs and/or display one or more system settings, according to some embodiments. FIG. 2 shows a non-limiting example graphical user interface 101 according to some embodiments. In some embodiments, the graphical user interface 101 is electronically coupled (e.g., wired or wirelessly) to a controller 102 which is configured to actuate one or more system components including one or more element fluid pumps 103.

In some embodiments, the one or more element fluid pumps 103 comprise one or more linear motors. In some embodiments, the one or more linear motors each include one or more coils 108, 109 and/or one or more cores 110, 111. In the non-limiting example shown in FIG. 1, the system includes a novel dual coil linear motor 103 that comprises a first coil 108 and a second coil 109 arranged along the same axis within a motor housing 112. In some embodiments, positioned within the first coil 108 and second coil 109 is a first core 110 and second core 111, respectively. In some embodiments, the first coil 108 is configured to move the first core 110 along a hollow axis of the first coil 108 through the generation of a first magnetic field. In some embodiments, the second coil 109 is configured to move the second core 111 along a hollow axis of the second coil 109 through the generation of a second magnetic field.

In some embodiments, the dual coil linear motor 103 is configured to actuate more than one piston 104, 105 (substantially) simultaneously. In some embodiments, a first drive shaft 113 with a first outer diameter is connected to the first core 110 and a first piston 104. In some embodiments, a second drive shaft 114 with a second drive shaft inner diameter is connected to a second core 111 and a second piston 105. In some embodiments, both the first piston 104 and second piston 105 are housed in the same element fluid pump 103.

In some embodiments, the second drive shaft inner diameter is defined by a second drive shaft hollow portion where the second drive shaft hollow portion extends axially along the second drive shaft 114. In some embodiments, the second core 111 includes a second core inner diameter defined by a second core hollow portion where the second core hollow portion extends axially along the second coil. In some embodiments, the second piston 105 includes a second piston inner diameter defined by a second piston hollow portion where the second piston hollow portion extends axially along the second piston 105.

In some embodiments, the second drive shaft inner diameter, the second coil inner diameter, and/or the second piston inner diameter are greater than the first outer diameter, such that the dual coil linear motor 103 is configured to enable the first drive shaft 113 to pass through the second coil 109, the second drive shaft 114, and/or the second piston 105 to connect to first piston 104 as shown in FIG. 1.

In some embodiments, the controller 102 is configured to control one or more fluid (e.g., gas) supply valves 115, 116 which are each configured to supply fluid to the pump configurations described herein from one or more fluid supplies 117. In some embodiments, the one or more fluid supplies 117 include one or more heater jackets 118 configured to regulate temperature via the controller 102. In some embodiments, such as the non-limiting example in FIG. 1, the controller 102 is configured to control a first fluid supply valve 115 and a second fluid supply valve 116. In some embodiments, the element fluid pump 103 includes one or more piston chamber housings 119 one or more pistons chambers 120, 121. In some embodiments, the element fluid pump 103 includes a first piston chamber 120 housing the first piston 104 and a second piston chamber 121 housing the second piston 105.

In some embodiments, the non-limiting example graphical user interface 101 is configured to display one or more system parameters including one or more fluid setpoints 201 and/or one or more sensor measurements 202. In some embodiments, the graphical user interface 101 is configured to display one or more element pressure setpoints 203 for one or more piston chambers 120, 121 and/or one or more elements 124, 125. In some embodiments, the graphical user interface 101 is configured to enable a user to enter one or more gas setpoints by selecting one or more icons shown in FIG. 2. In some embodiments, the graphical user interface 101 is configured to enable a user to enter one or more fluid setpoints for the first piston chamber 120 and/or the second piston chamber 121. In some embodiments, a gas setpoint includes one or more of a pressure, a flowrate, a temperature, and/or an actuation status. In some embodiments, the graphical user interface is configured to display and/or control one or more of electrocardiogram (ECG), heart rate, inlet blood pressure, outlet blood pressure, flow rate, cycle settings, active cycle rate, gas pressure settings each element. active gas pressure each element, error codes, alarm, pump identification (ID), date, time, run time, operator ID, patient ID and/or asset location.

In some embodiments, a reciprocating action of a piston 104, 105 is configured to increase pressure and/or force fluid to one or more elements 124, 125 by decreasing the volume of at least a portion of a piston chamber. In some embodiments, the reciprocating action of a piston 104, 105 is configured to decrease pressure and/or retrieve gas from the one or more elements by increasing the volume of at least a portion of a piston chamber. In some embodiments, the controller 102 is configured to maintain a substantially constant mass of fluid within a piston chamber 104, 105. In some embodiments, the GUI 101 is configured to enable a user to enter a pressure range and/or display one or more warnings/and or alerts on the GUI 101 if the range is exceeded. In some embodiments, the controller 102 is configured to initiate emergency pressure control instructions configured to remove any fluid from a first fill lumen 122 and/or second fill lumen 123 and/or stop the operation of a piston 104, 105.

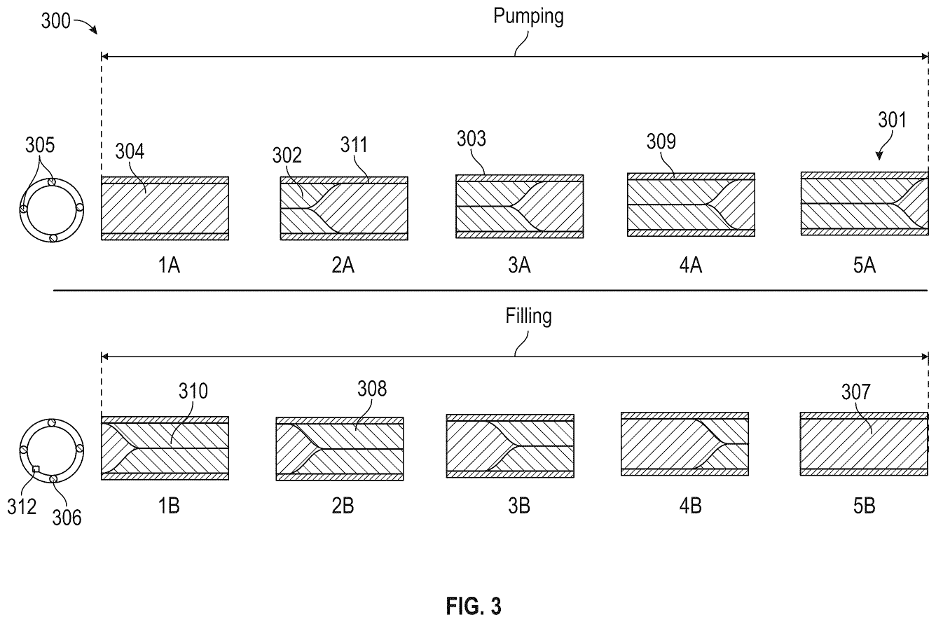

FIG. 3 shows an element filling operation 1A-5B for the heart assist pump 106 executed by the system in a tubular element arrangement 300 according to some embodiments. In some embodiments, as shown in the non-limiting example in FIG. 3, the heart assist pump 106 includes one or more elements 301, which can be located in the aorta pump section 2901 and/or the LV pump section 2902 formed by the hour-glass shaped stent 107. In some embodiments, the stent 107 may take on a non-hour-glass configuration such as those described in relation to FIGS. 47-58 when configured for placement in the descending aorta. Any tube or catheter described herein can be used in conjunction with any pumping element and/or combination of pumping elements described herein.

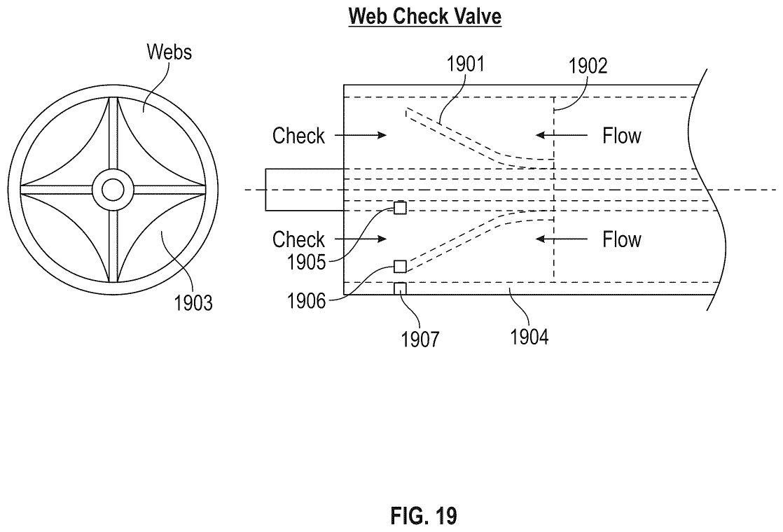

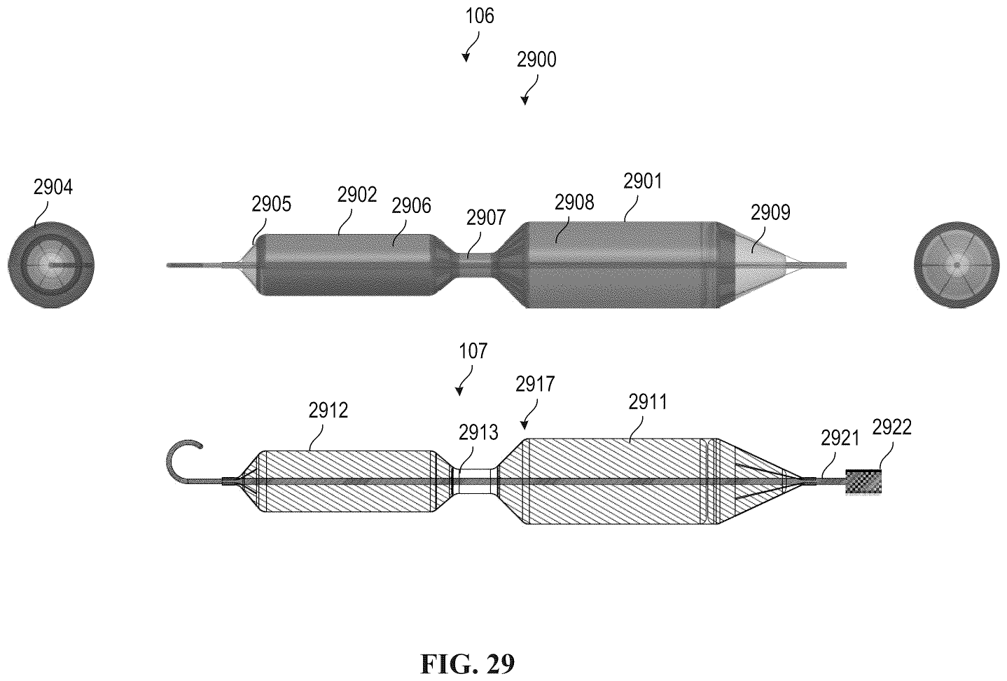

In some embodiments, the heart assist pump 106 includes a tube 303. In some embodiments, the element 301 includes a single balloon 302 positioned within at least a portion of the stent 107 and/or tube 303. In some embodiments, the length of the tube 303 extends past the length of the balloon 303. In some embodiments, a stent 107 surrounds the balloon as shown in FIG. 29. In some embodiments, the tube 303 is configured to transport a liquid 304 such as blood, as a non-limiting example. In some embodiments, one or more fill lumens 305 supplying fluid from one or more element fluid pumps 103 connect to the single balloon 302 by extending through a fill tube 305 to the balloon 302 to actuate a filling and pumping operation of the balloon 302. In some embodiments, a plurality of fill lumens 305 supplied to a single balloon 302 provides the benefit of applying pressure to a balloon 302 from a plurality of directions. In some embodiments, the tube 305 includes a catheter, such as catheter 1904 as described in relation to FIG. 19.

In some embodiments, each of the one or more fill lumens 305, which may include fill lumens 122, 123 comprise one or more fill orifices 306 configured to deliver the element fluid to one or more balloons 302. Although FIG. 3 depicts a single balloon 302, as further described herein the system comprises a plurality of balloons (elements) in series as shown in FIG. 29 according to some embodiments. Any property of an element described according to some embodiments is understood to be applicable to any other element arrangement (e.g., plurality arrangement) according to some embodiments. In some embodiments, each of the one or more fill lumens 305 are configured to supply fluid to a separate balloon in a plurality arrangement as described later. In some embodiments, one or more fill lumens 305 are configured to supply fluid to a first balloon of a plurality of balloons, and one or more other fill tubes are configured to supply fluid to one or more other balloons of the plurality of balloons. In some embodiments, the heart assist pump 106 is configured to at least partially trap and/or transport a fluid in a bi-directional manner by actuating one or more balloons in the series, with non-limiting examples of a bi-directional arrangement illustrated in FIGS. 47-58.

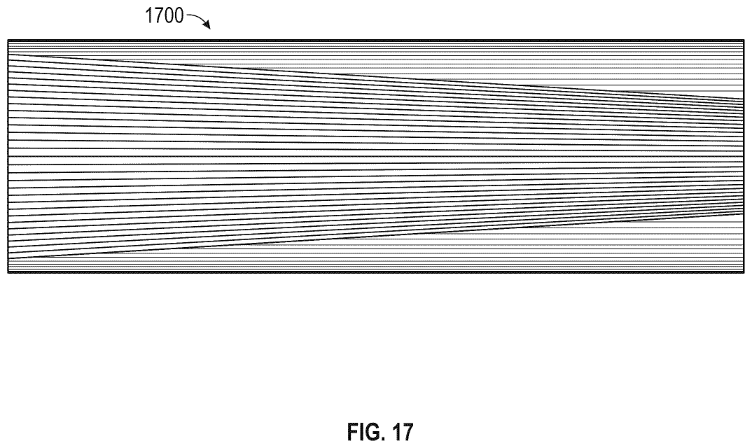

Referring back to FIG. 3, in some embodiments, one or more balloons 302 comprise a balloon hollow core 307 which give the balloon a tubular shape when at least partially deflated at a first pump stage 1A, which is the same as a final pump stage 5B. In some embodiments, one or more balloons 302 are configured to directionally inflate. In some embodiments, the one or more balloons each comprise one or more different densities and/or thicknesses in at least a portion a balloon wall along the balloon's length. FIG. 17 illustrates an exaggerated density profile according to some embodiments. In some embodiments, at least a portion of a balloon's outer wall 308 is attached to the fluid conduit 309. In some embodiments, directional inflation and/or directional deflation cause a pumping action of fluid within at least a portion of the fluid conduit 309.

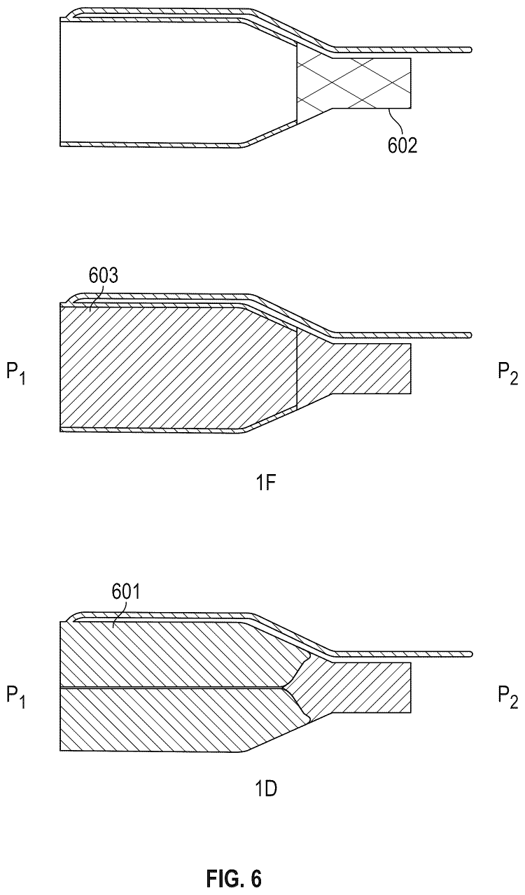

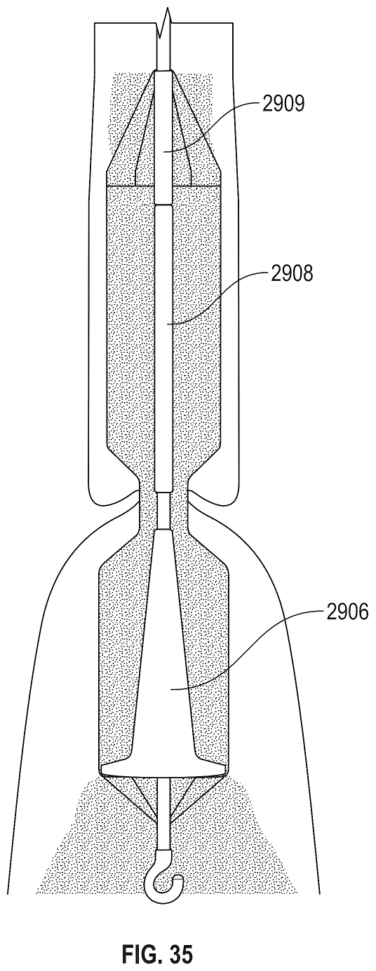

In some embodiments, upon an actuation of a piston 104, the balloon 302 is configured to at least partially seal a first end of the fluid conduit 309 (see FIG. 35), which may also include and/or be an element housing, by pressing the balloon inner faces toward each other at a stage 2A. FIGS. 6-12 provide an enlarged view of this action according to some embodiments, noting that features from any of the shown figures in all portions of this disclosure are interchangeable and readily incorporable with each other. Stages 3A-5A show continued expansion of the balloon 302 pushing the fluid to the right according to some embodiments. In some embodiments, this increases the pressure of the moving fluid greater than the pressure being supplied by incoming fluid from the left. In some embodiments, as a piston return stroke starts to remove pressure from the balloon 302 the incoming fluid from the left pushes balloon inner faces 310 back toward the fluid conduit wall 311 as the piston pulls the gas from one or more fill orifices 306. In some embodiments, one or more balloons, one or more fill tubes, and/or one or more fluid conduits comprise one or more sensors 312 (e.g., Wheatstone bridge) configured to feedback one or more balloon parameters to the controller in the form of electrical signals, electromagnetic signals, light signals, and/or fluid pressure signals. In some embodiments, a balloon parameter includes one or more of a balloon pressure, a balloon tension, a balloon activation, a percent inflation, and/or any other parameter that can be interpreted from a signal from a sensor.

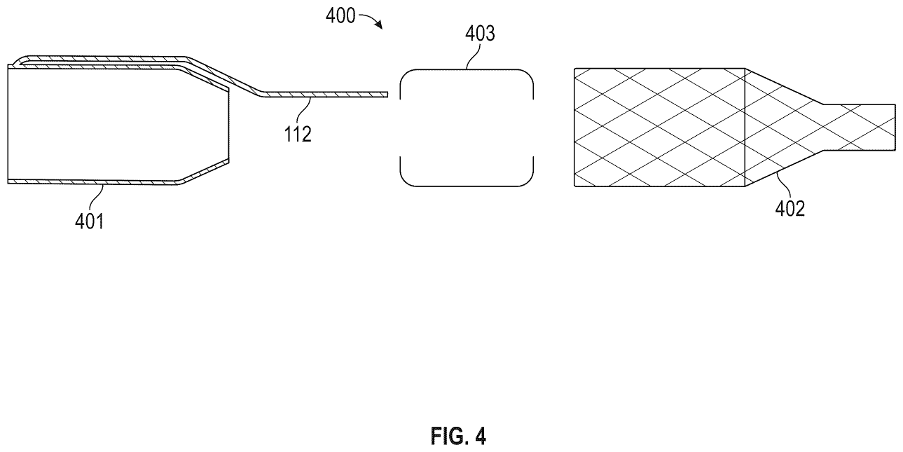

As shown in FIG. 4, in some embodiments, the heart assist pump 106 includes a stent arrangement 400 comprising one or more elements 401 and one or more stents 402 (e.g., nitinol stents as a non-limiting example). The stent arrangements in FIGS. 4-15 represent half of heart assist pump 106 stent 107, where the description and/or arrangement of the elements in any figure can be applied to either or both sides. In some embodiments, size is a limiting factor for the heart assist pump 106. In some embodiments, the stent arrangement 400 is configured and/or sized to be inserted into a human body. In some embodiments, one or more check elements (e.g., balloons, magnetic valves) are minimized in size to enable a larger main element which can induce a larger flow as further described below.

In some embodiments, the system comprises one or more elements 124, 125 within the stent 107. In some embodiments, the system comprises an element housing 403 (309), where the one or more elements 401 are located within the element housing 403. In some embodiments, the element housing 403 is configured to be placed within the stent section 402. In some embodiments, the element housing 403 is flexible and/or collapsible. In some embodiments, the element housing 403 and/or the one or more elements 401 are configured to be removed from the stent after the stent is placed inside a patient. In some embodiments, the element housing is coupled to a mandrel. In some embodiments, the element housing is coupled to the stent section 402. In some embodiments, the element housing 403 includes an integrated stent, which forms a tubular stent. In some embodiments, the element housing 403 is configured to trap fluid within and/or direct fluid through the heart assist pump 106.

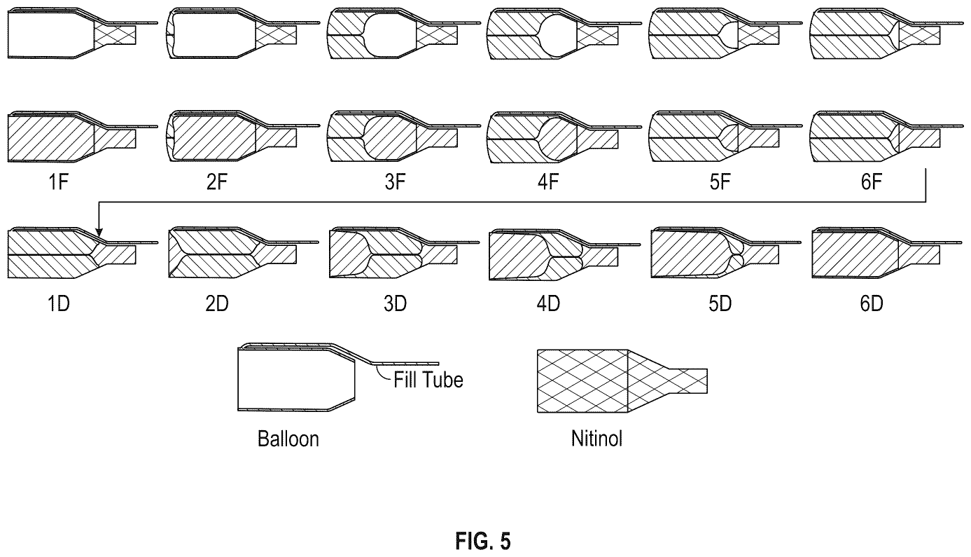

In some embodiments, stent 107 comprises a temperature dependent memory metal configured to expand when exposed to body temperature for a pre-determined time. FIG. 5 illustrates the operation of the stent arrangement 400 which is similar to the operation of the balloon within the tube arrangement 300 previously described according to some embodiments. Enlarged details of the stent arrangement 400 according to some embodiments can be seen in FIGS. 6-15.

While not to be limited to any principle or application of physics, a discussion of the operation of a stent (and/or tube) arrangement for a single element and/or portion of a plurality of elements is described below. Inflation and deflation steps are illustrated together to highlight the behavior of the balloon structure under opposite conditions.

In some embodiments, systems and methods described herein are directed to a number of cycles (e.g., per min) needed to achieve a desired flow rate while delivering a desired output pressure given a fixed pumping volume per cycle. In some embodiments, factors other than the fixed volume of the pumping element that influence cycle time include the fluid pressure required to inflate each element and the time to deflate each element. In some embodiments, the higher the final inflation pressure, the longer the deflation time given the use of dual function lumens, which perform both inflation and deflation functions. In some embodiments, one area that affects the pressure requirement is loss in the lumen due to the size of the lumens. In some embodiments, empirical data has revealed that approximately 40% of the overall element fluid lumens area allocated to each pumping element, approximately of 10% to the inlet check element, and approximately of 10% of the overall gas fill lumens to the outlet check element, is sufficient to achieve the desired pumping effect. Although the function of a check element is to prevent the flow of liquid, the inflation action of a check element, which may include a directional inflation, also provides pumping action according to some embodiments.

FIG. 6 shows a 1st filling step 1F and a 1st deflating step 1D in a balloon cycle according to some embodiments. In some embodiments, these two steps represent when an element 601 is at a completely deflated and fully inflated configuration, respectively. In some embodiments, when completely deflated at step 1F, the pressure on either side of the stent 602 P₁ and P₂ are equal, but generally lower than desired in the implant environment. In some embodiments, after the liquid 603 has been forced completely from the element 601 and/or stent 602, P₁ is slightly higher than P₂ at step 1D as pressure builds at the opening.

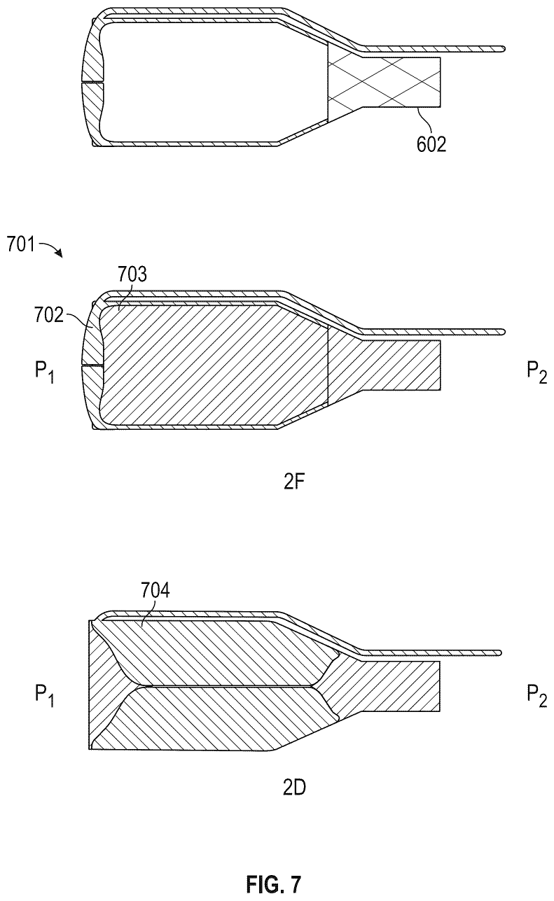

FIG. 7 shows a 2nd filling step 2F and a 2nd deflating step 2D according to some embodiments. In some embodiments, at a 2nd filling step the upstream opening 701 is closed by the element gate portion 702 trapping the liquid 703 in the balloon's central portion. In some embodiments, at a 2nd deflating step the balloon gate portion 702 is deflated before the balloon body portion 704. In some embodiments, the deflation of the gate portion 702 first is at least partially aided by one or more of the element's structure (e.g., density) and the higher pressure at P1.

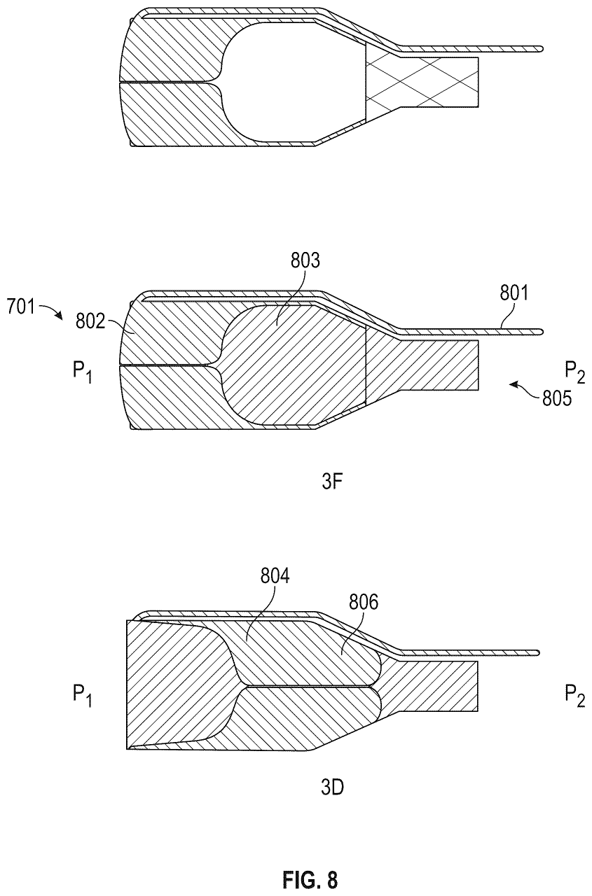

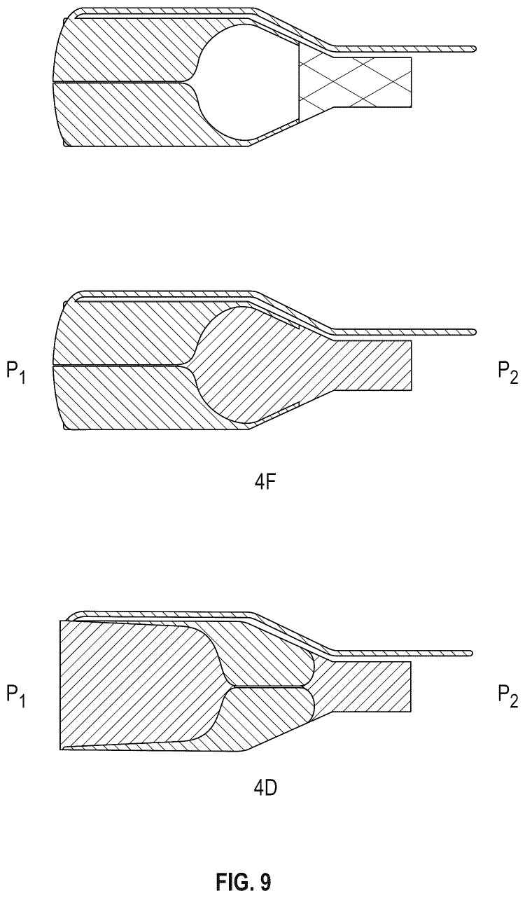

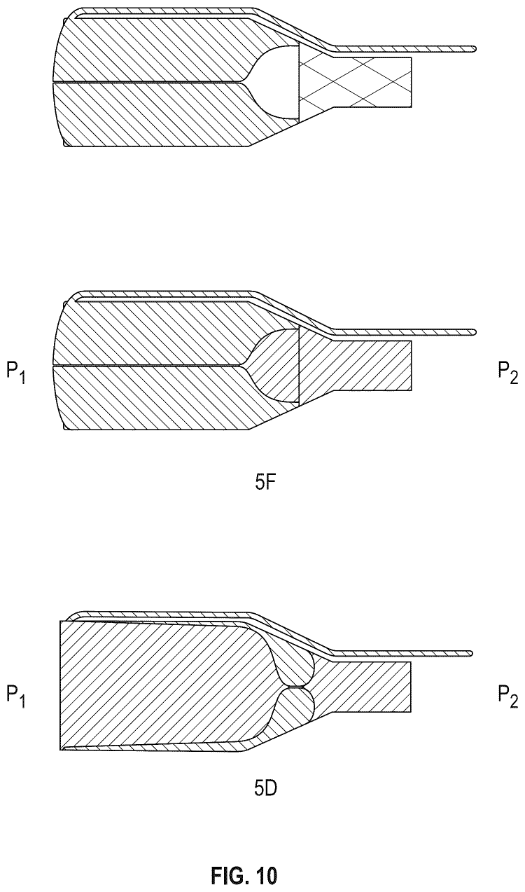

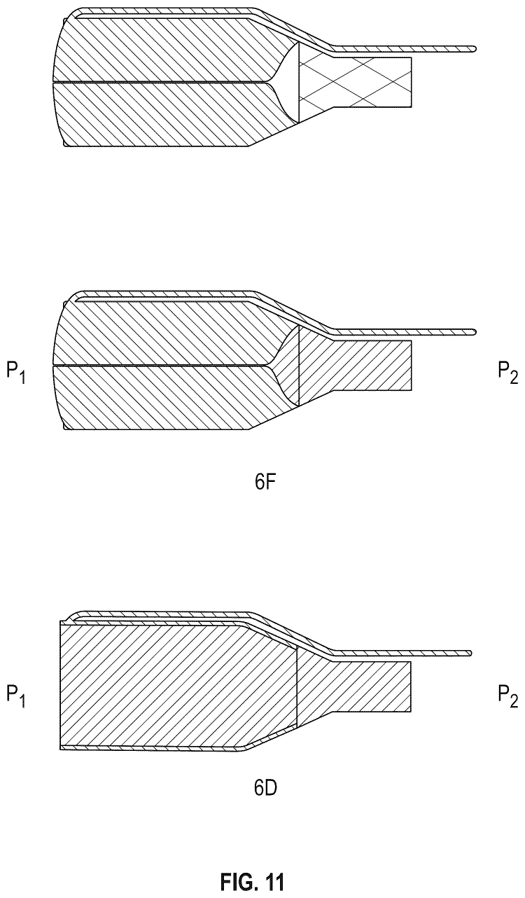

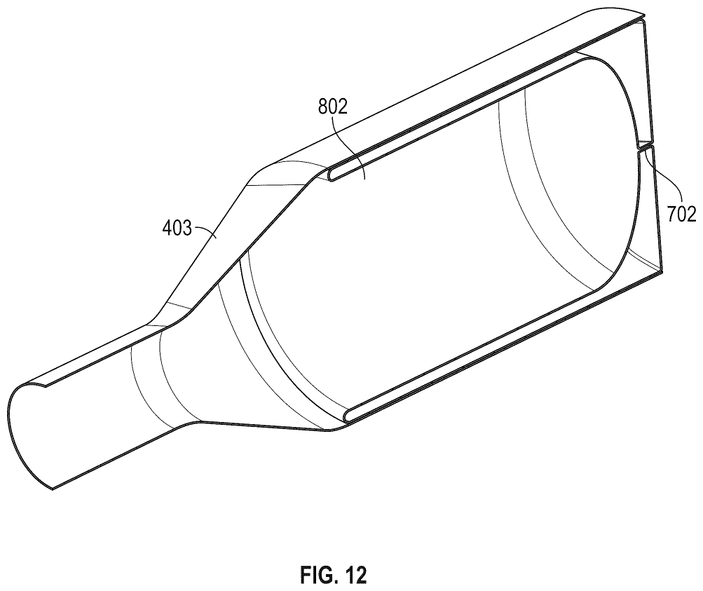

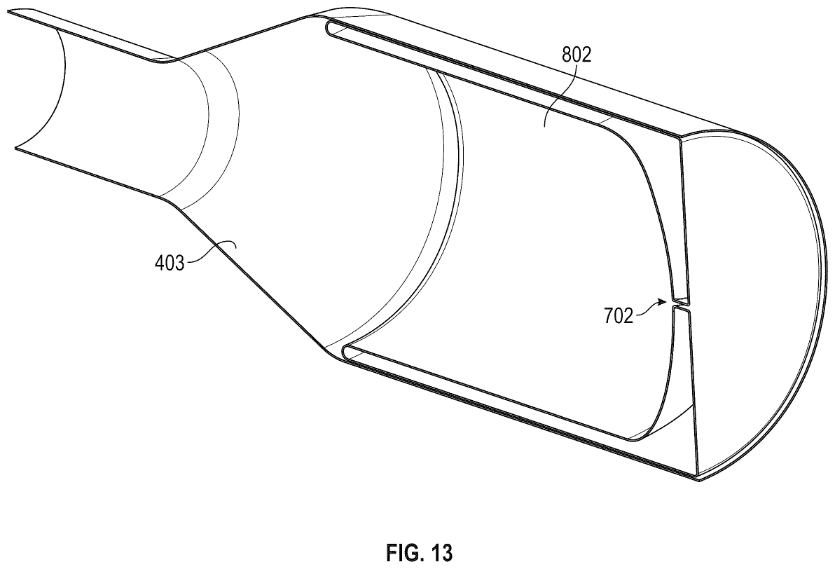

FIG. 8 shows a 3rd filling step 3F and a 3rd deflating step 3D according to some embodiments. In some embodiments, the pressure of fluid supplied from the fill tube 801 and/or the structure of the element at filling step 3F causes the element body portion 804 to inflate toward the downstream opening 805. In some embodiments, this inflation direction forces the liquid 803 out of the downstream opening increasing the overall pressure downstream. In some embodiments, at a 3rd deflation step 3D, the upstream pressure P₁ pushes the liquid 803 against the balloon body portion 804 which pushes element fluid 806 toward the downstream opening end maintaining a closed balloon configuration at the downstream opening 805. FIG. 9 is a continuation of the inflation and deflation illustrations of FIG. 8. FIG. 10 is a continuation of the inflation and deflation illustrations of FIG. 9. FIG. 11 shows a final inflation step 6F and a final deflation step 6D, which is also the beginning of the cycle as shown in FIGS. 5 and 6 according to some embodiments. FIGS. 12 and 13 show a non-limiting rendering of an element configuration according to some embodiments. Although the inflation direction is shown coming from the wider end of the element, in some embodiments the inflation direction is the opposite direction. In some embodiments, the element is configured to use the narrower section of the element housing and/or stent to force the element end to close first, aiding the directional inflation.

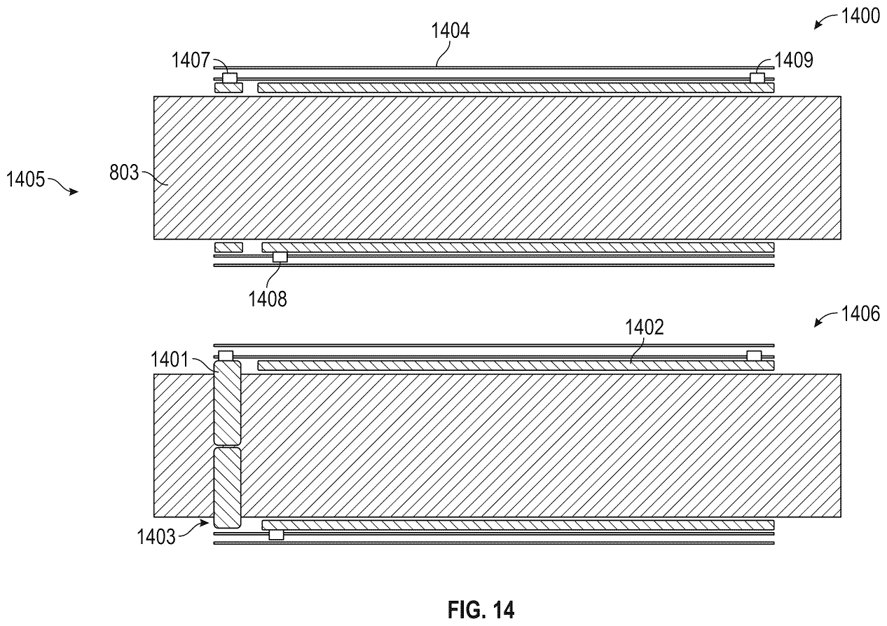

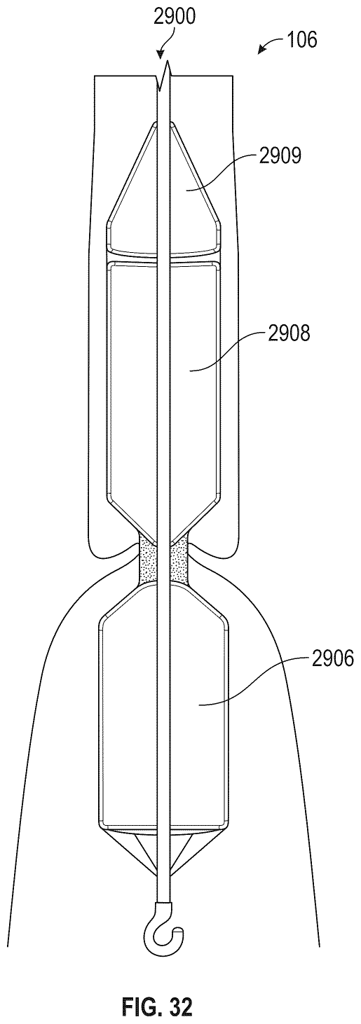

FIG. 14 shows a plural balloon system 1400 comprising two elements 1401, 1402 in this non-limiting example: note that the same directional inflation operation in this tube arrangement applies to the stent arrangement shown in FIGS. 32-38, which illustrates the interchangeability of the different arrangements described herein. In some embodiments, the controller 102 is configured to inflate the first element 1401 before inflating the second element 1402. In some embodiments, the controller 102 is configured to inflate the second element 1402 when the first element 1401 at least partially seals a first end 1403 of the tube 1404, thereby preventing further fluid flow from upstream 1405 (e.g., in the direction of natural blood flow) in this non-limiting example. In some embodiments, the controller 102 is configured to inflate the second balloon in a directional manner previously described with respect to FIGS. 3 and 5. In some embodiments, the natural downstream flow 1406 aids in the formation of the balloon as previously described.

In some embodiments, one or more balloons includes a single orifice 1407. In some embodiments, the one or more balloons include two or more orifices 1408. In some embodiments, a tube (element housing) 1404 may comprise one or more lumens, which may be embedded in the wall of the tube 1404 and/or couple to the tube 1404. In some embodiments, the second element 1402 comprises an upstream orifice 1408 and a downstream orifice 1409. In some embodiments, controller is configured to apply positive pressure to the upstream orifice 1408 while applying a negative pressure to the downstream orifice 1409. In some embodiments, a negative pressure is configured to hold the internal walls of the second element 1402 against one another while the positive pressure fills the upstream portion of the second element 1402.

In some embodiments, cycle time is influenced by the dwell time in between each element's actuation action driven by inflation time and deflation time before the next element can activate. In some embodiments, a 1 to 100 millisecond overlap between actuation of individual elements has been determined to ensure smooth flow with little to no back flow while decreasing cycle time. In some embodiments, the system is configured to begin inflating a pump element 1402 before the inflation of a check element 1401 is complete. In some embodiments, the system is configured to begin the inflation of a check element 1401 before a pump element 1402 has been fully inflated. In some embodiments, the system is configured to begin deflating a pump element 1402 before the deflating of a check element 1401 is complete. In some embodiments, the system is configured to begin the deflating of a check element 1401 before a pump element 1402 has been fully deflated.

In some embodiments, the system is configured to optimize a sequence of inflation and deflation for each element to produce a cycle time for the device that supports a target flow rate when given a targeted device size. In some embodiments, the system is configured to vary and/or supply different element fluid pressures to each element to be able to allow each element to perform the inflation and deflation in a way that reduces cycle time.

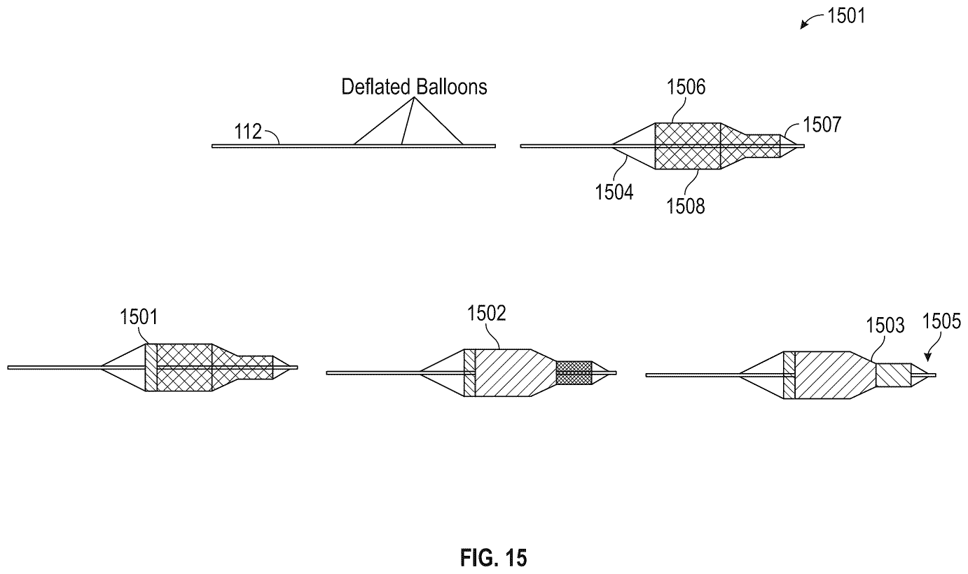

FIG. 15 shows a three-balloon arrangement 1500 for various configurations of the of heart assist pump 106, such as those described in relation to FIGS. 29-58, in accordance with some embodiments. In a non-limiting example where element 1501 is the inlet check element and element 1503 is the outlet check element, both are synchronized with pumping element 1502. In some embodiments, pumping element 1502 comprises an inflated volume at least twice that of check element 1501 and/or 1502. In some embodiments, in each case the inlet check element 1501 and outlet check element 1502 must execute their function to allow the pumping element 1502 to perform in a sequential way which generally results in reduced fluid movement time in the cycle. In some embodiments, the system is configured to supply different pressure settings to each element. In some embodiments, the system is configured to supply higher pressure to the check elements to create a quicker inflation time. In some embodiments, due to different inflated volumes of each element 1501, 1502, and/or 1503 (or any element described herein), the opportunity for overlapping activation exists by utilizing the pressurization time of the pumping element against the shorter inflation and deflation time of the check/pump elements. The check elements also provide some pumping action as a result of reducing volume according to some embodiments.

In some embodiments, the system is configured to apply a vacuum to one or more elements. In some embodiments, the vacuum assist deflation which influences cycle time which can be used to control cycle time of each element. In some embodiments, the system is configured to generate a maximum vacuum (i.e., highest the system will allow) to shorten the cycle time.

In some embodiments, one or more fill lumens have a dual function of delivering fluid at a set pressure and removing fluid under vacuum. In some embodiments, the size of the fluid passage influences the cycle time. In some embodiments, each element's size affects available volume to pump fluid (e.g., blood) and, by minimizing the size of the check/pump elements, the volume pumped per cycle increases. In some embodiments, empirical testing has shown an inlet check element at a maximum of 15% inflated volume of the pumping element inflated volume, and an outlet check valve of a maximum of 10% of the pumping element inflated volume produces acceptable results.

In some embodiments, when inserted to a specified location in a human body approximately the controller 102 is configured to apply 20-30 mmHg of positive pressure exist on the large end of the balloon housing. In some embodiments, the controller 102 is configured to inflate each element in a sequence that causes a decrease in volume and displacing the fluid through the small end 1507 of the element housing and/or stent 1508. In some embodiments, the controller 102 is configured to deflate the elements in a sequence, drawing fluid into the element housing filling at least a portion or the entirety of each void left by a balloon's deflation.

In some embodiments, the three-balloon arrangement 1501 (or any 3 element arrangement described herein) comprises a central pumping balloon 1502 that has an expanded volume greater than an upstream check element 1501 and/or a downstream check element 1502. In some embodiments, a pumping sequence starts with the controller 102 inflating all elements. In some embodiments, the controller 102 is configured to deflate the pumping balloon 1502 to create a vacuum. In some embodiments, the controller 102 is configured to deflate the upstream check balloon 1501 (e.g., in approximately 1/10 second or less) to create an additional vacuum. In some embodiments, the (combined) vacuum draws fluid (e.g., blood under 20-30 mmHG of positive pressure) into the balloon housing 1506. In some embodiments, the controller 102 is configured to deflate the downstream check element 1503 (e.g., in approximately 1/10 second or less and/or while the fluid is in motion) adding additional momentum from the vacuum created to eject the fluid from the balloon housing. In some embodiments, the momentum this arrangement provides creates a “slingshot effect” that results in greater fluid volume output than the sum of the balloon volumes and/or balloon housing volume. In some embodiments, the controller 102 is configured to repeat these initiation instructions one or more times to create a pumping cycle. In some embodiments, the repeating action creates a generally or completely sinusoidal wave fluid flow and/or fluid pressure profile. In some embodiments, the controller 102 is configured to initiate the inflating and deflating sequence in an opposite manner such that the downstream check element 1503 deflates before the upstream check element 1501 deflates thereby enabling flow in a bi-directional manner. In some embodiments, a controller-initiated pump sequence can include program instructions for a right-to-left and/or left-to-right inflation/deflation sequence for any combination of elements described herein where each element is inflated and/or deflated in a sequential order. In some embodiments, the GUI is configured to enable a user to program the controller to inflate and/or deflate one or more elements in any sequence described herein.

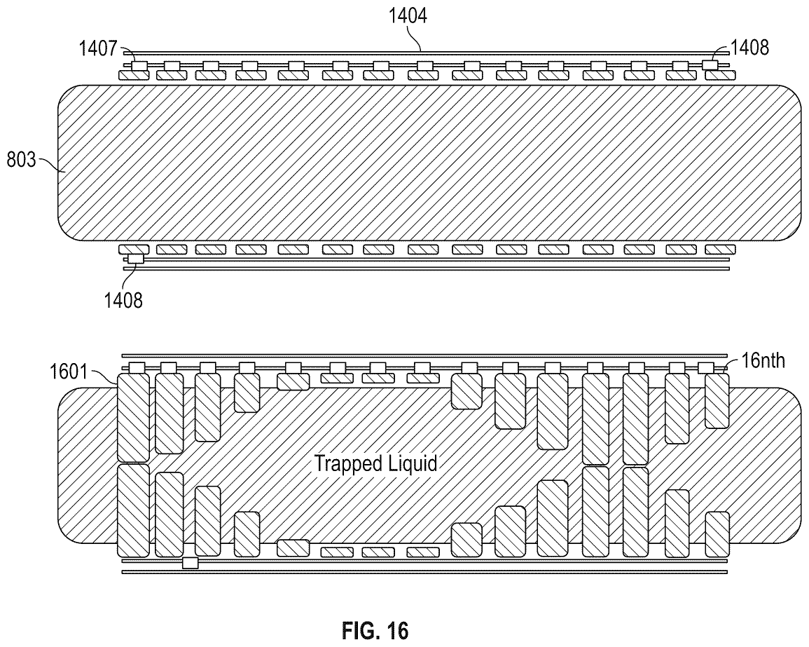

FIG. 16 shows a sequential element arrangement 1600 comprising a plurality of elements 16nth according to some embodiments. In some embodiments, each element 1601-16nth is approximately the same size. In some embodiments, one or more elements are different sizes and/or displace different volumes. In some embodiments, each element can exhibit the same or very different inflation characteristics depending on wall thicknesses, durometers, and other typical balloon characteristics. In some embodiments, the controller 102 is configured to acuate one or more elements, which include a first element 1601 up to an nth element 16nth (i.e., any number of elements) in a peristalsis sequence where trapped fluid is moved along a path by supplying fluid (e.g., gas) in a predetermined order. Although, in this example, the fluid is trapped between two or more fully inflated elements, in some embodiments one or more of the plurality of elements are configured to directionally inflate in a similar shape and flow profile as previously described with respect to a single, dual, and/or multiple element arrangements previously described and illustrated in at least FIGS. 29-58.

In some embodiments, the controller 102 is configured to generate 20-30 mmHg of vacuum at the distal (larger diameter) end of the peristalsis heart assist pump. In some embodiments, the controller 102 is configured to initiate a pumping cycle similar to those previously described herein according to some embodiments, such as with the three-element arrangement. In some embodiments, the controller 102 is configured to implement a sequence to generate fluid momentum by inflating and deflating the elements in an order as previously described.

In some embodiments, an element shape when deflated and/or inflated influences pumping action in a pumping element and/or fluid element. In some embodiments, a pumping element shape allows for the inflation to occur in a desirable direction which results in the fluid flow moving from inlet to outlet in a more controlled way.

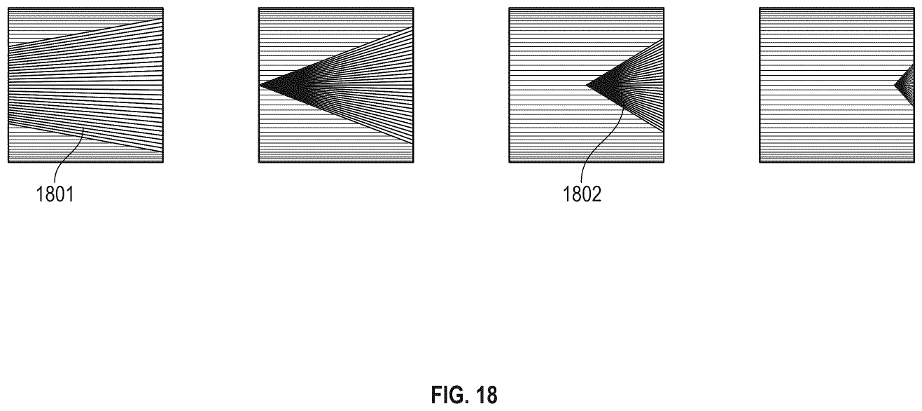

In some embodiments, one or more elements described herein include a tapered shape and/or are configured to inflate in a tapered shape. FIG. 17 shows a tapered shaped element 1700 according to some embodiments. In some embodiments, the tapered shape is configured to create a cone shaped volume pocket 1801 during inflation. FIG. 18 illustrates the cone shaped profile 1801 according to some embodiments. As shown in FIG. 18, in some embodiments, as this pocket 1801 collapses during inflation the angle 1802 of the cone increases as more of the inflated balloon fills the inner portion of a tube, stent, and/or element housing. In some embodiments, the tapered shape of the tapered element is configured to increase the velocity of the trapped fluid as the trapped fluid is moved from the inlet to the outlet end as the pocket 1801 collapses. In some embodiments, the increased velocity results in an increase in fluid pressure. In some embodiments, a tapered element 1700 which causes inflation to be directed to one direction is desirable in achieving a controlled flow and reduces high velocity areas in the fluid flow. In some embodiments, empirical data has shown taper angle range of 1 degree up to 60 degrees at any point along 80% of the element length which will achieve a desirable result. In some embodiments, one or more check elements and/or pump elements described herein include a tapered element 1700. In some embodiments, a tapered element with a pleated configuration is described below.

In some embodiments, the check/pump element shapes are configured to inflate in a substantially perpendicular direction with sufficient surface area force to at least partially seal the element housing with minimum gas volume. In some embodiments, element area ribbing is configured to direct inflation while limiting expansion to enable increased pumping element capacity. In some embodiments, check element inflation is configured to provide pumping action. In some embodiments, the pumping action is bidirectional.

In some embodiments, element material thickness, durometer value, and/or ribbing surface are configured to provide benefit in controlling inflation and/or configured to provide additional benefit of encouraging the delated element against a mandrel to decrease deflated volume. In some embodiments, a tapered thickness of material for the pumping element provides similar benefits for controlling flow direction by having the thinner material area inflate first, resulting in the controlled directional inflation (see FIGS. 17 and 18). In some embodiments, material thickness differential of minimum 5% to 300% in a taper or intermittent fashion along the element has been empirically determined to provide a desired effect.

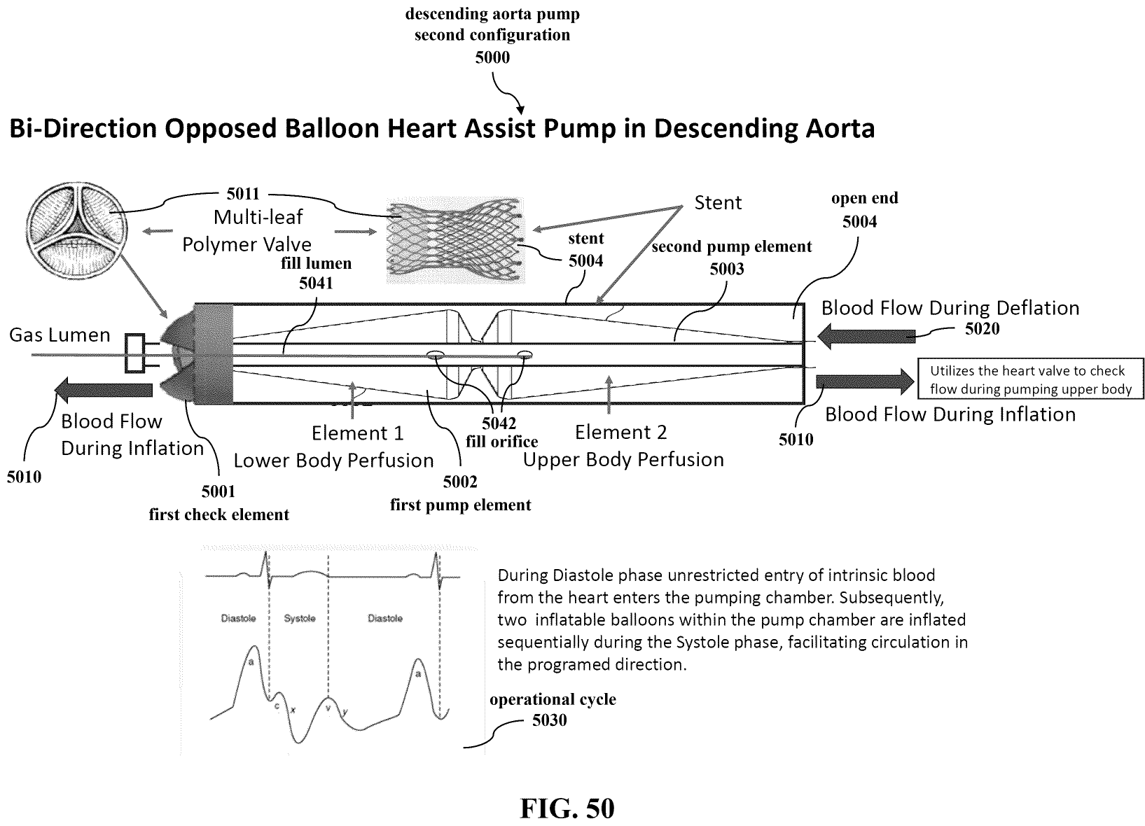

FIG. 19 shows one or more check elements 1901 according to some embodiments. In some embodiments, one or more check elements 1901 are not balloons. In some embodiments, the check elements 1901 include a multi-leaf (web) configuration, such as the non-limiting example pump configurations of FIGS. 50-58. In some embodiments, the one or more check elements 1901 are configured to curve upward and/or block liquid flow in one direction. In some embodiments, the one or more check elements 1901 are configured to enable flow in one direction. In some embodiments, the one or more check elements 1901 are configured to be pushed down by one or more inflating balloons, such as a pump element in a single balloon arrangement. In some embodiments, one or more check elements are a combination of balloons and web/leaf elements. In some embodiments, the one or more check elements 1901 are configured to cause at least a portion of the one or more balloons 1902 to lift and/or interfere with a direction of flow while allowing flow in the other direction when deflated. In some embodiments, the one or more check elements 1901 are the same material as the balloon 1902. In some embodiments, the one or more check elements 1901 are a different material as the balloon 1902. In some embodiments, the one or more check elements 1901 include a different density as the surrounding material. In some embodiments, the heart assist pump 106 comprises one or more balloons 1902 and/or one or more check elements (e.g., 1-2 elements) 1901. In some embodiments, the one or more check elements 1901 are configured to flatten during positioning into a patient and/or expand to a functional configuration once positioned in a patient.

In some embodiments, the one or more check elements 1901 include one or more expandable portions 1903 configured to be inflated by an element fluid. In some embodiments, the one or more expandable elements 1903 are housed in a stent 107. In some embodiments, the one or more expandable elements 1903 are configured to act as a check valve. In some embodiments, the one or more expandable elements 1903 are configured to act as a check valve when deflated. In some embodiments, the stent 107 comprises a nitinol stent. In some embodiments, one or more stents 107 are housed inside a catheter 1904, which may also be a lumen and/or tube according to some embodiments. In some embodiments, the one or more stents 107, check elements 1901, 1501 element housings 403, and/or elements 16nth are configured to project from the catheter 1904 once in position. In some embodiments, system is configured to enable the one or more stents 107, check elements 1501, element housings 403, and/or elements 124, 125 to be collapsed, rehoused, and/or be crimped back inside the catheter 1904 during extraction from the patient.

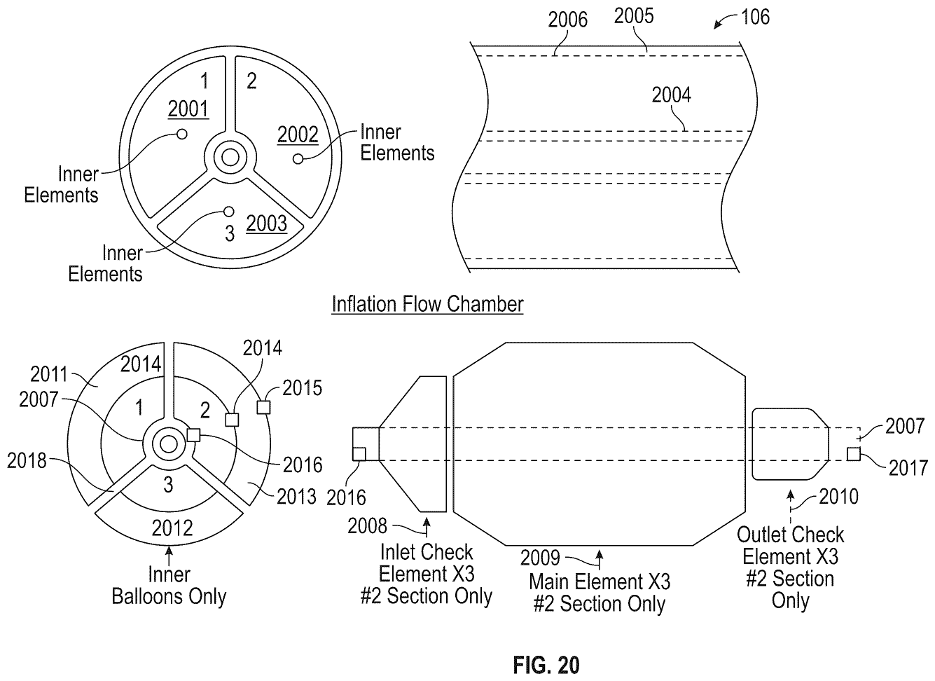

FIG. 20 shows a heart assist pump 106 that includes a mandrel flow chamber according to some embodiments. In some embodiments, the flow chamber includes one or more inflatable elements 2001, 2002, 2003 according to some embodiments. In some embodiments, the flow chamber includes one or more fill lumens 2004, 2005 incorporated into the flow chamber walls 2006 and/or the mandrel 2007. In some embodiments, element fluid is delivered from the mandrel to the flow chamber walls 206 via one or more arms 2018, which may include stent arms. In some embodiments, the one or more check elements 2008, 2010 and/or one or more pump elements 2009 are configured to be attached and/or are coupled to the inside of the flow chamber wall 2006 and/or the mandrel 2007. In some embodiments, the one or more check elements 2008, 2010 and/or one or more pump elements 2009 are configured to inflate toward the center of the flow chamber 2011. In some embodiments, the one or more check elements and/or one or more pump elements are configured to inflate downward and/or away from the flow chamber wall 2006.

In some embodiments, the flow chamber 2011 comprises a mandrel 2007. In some embodiments, the one or more check elements 2008, 2010 and/or one or more pumping elements 2009 are configured to be attached and/or are coupled to the mandrel 2007. In some embodiments, the mandrel 2007 includes one or more arms 2018 extending the inner diameter of the flow chamber 2011. In some embodiments, the one or more arms 2018 are each configured to form a separate chamber 2012, 2013, 2014 in the flow chamber 2011. In some embodiments, the one or more check elements 2008, 2010 and/or one or more pumping elements 2001, 2002, 2003 are configured to inflate upward and/or outward from the mandrel 2007 to the flow chamber wall 2006. FIG. 20 shows both arrangements where regions 1, 2, and 3 are either the elements inflating outward or the void as the elements inflate toward the mandrel.

In some embodiments, the catheter 1904 is configured to couple to the heart assist pump 106. In some embodiments, the heart assist pump 106 includes a plurality of fill lumens 2004 (and/or 2005) configured to feed element fluid (e.g., gas, liquid) to the one or more check elements 1501, 1503 and/or one or more pumping elements 1502. In some embodiments, the catheter 1904 and/or mandrel 2007 are configured to function as a guide for wire and/or an access for wire. In some embodiments, the one or more check elements 1501, 1503 and/or one or more pumping elements 1502 are configured to be inflated using one or more pressurized gas cylinders, valves, and/or compressed gas sources.

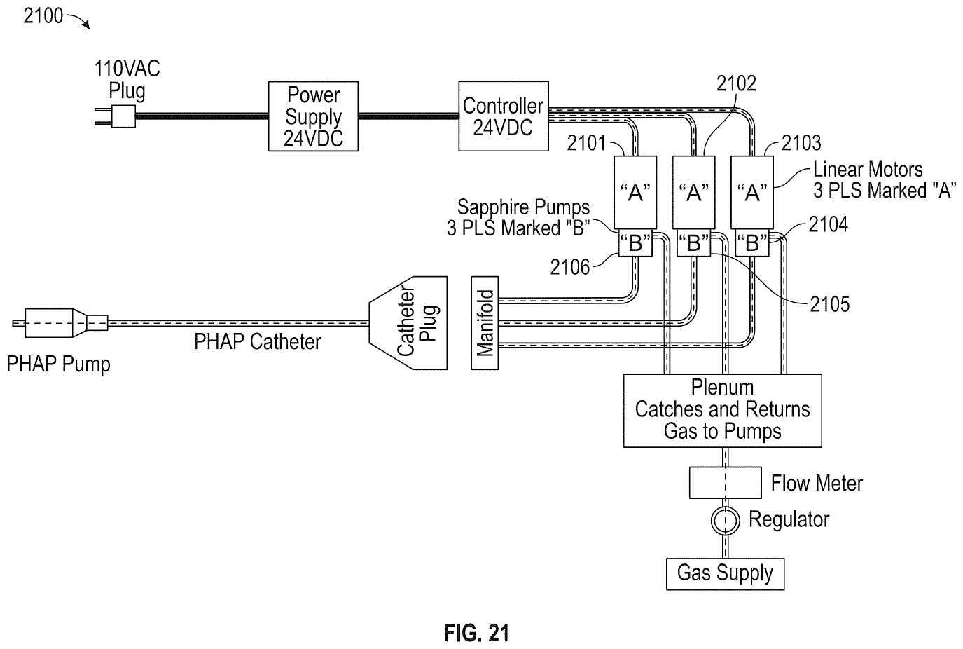

FIG. 21 shows a multi-element fluid pump arrangement 2100 according to some embodiments. In some embodiments, the system includes a plurality of element fluid pumps 2101, 2102, 2103 that are configured to inflate and/or deflate one or more check elements 1501, 1503 and/or one or more pump elements 1502 as previously described. In some embodiments, one or more pump pistons 2104, 2105, 2106 comprise sapphire and/or a similar material manufactured to a tolerance that needs no valve seals to enable the element fluid pumps 2101, 2102, 2103 to create pressure on a forward cycle and/or a vacuum on the reverse cycle. In some embodiments, the plurality element fluid pump arrangement 2100 and/or the dual linear pump 103 arrangement comprise a closed loop system configured to recirculate and/or reuse element fluid in a cyclical manner.

In some embodiments, the system comprises one or more linear motors, rotary motors, linear motion devices, and/or rotary motion devices configured to power one or more element fluid pumps 2101, 2102, 2103 to inflate and/or deflate various aspects of the system according to some embodiments as previously described. In some embodiments, one or more element fluid pumps 2101, 2102, 2103 are configured to generate a vacuum to increase the speed of deflation. Any number of element fluid pumps may be used to feed a respective number of lumens, or a single element fluid pump may be used to fill a plurality of elements through a single lumen, where the shape and/or material properties of each element determines a relative timing for inflation and/or partial inflation.

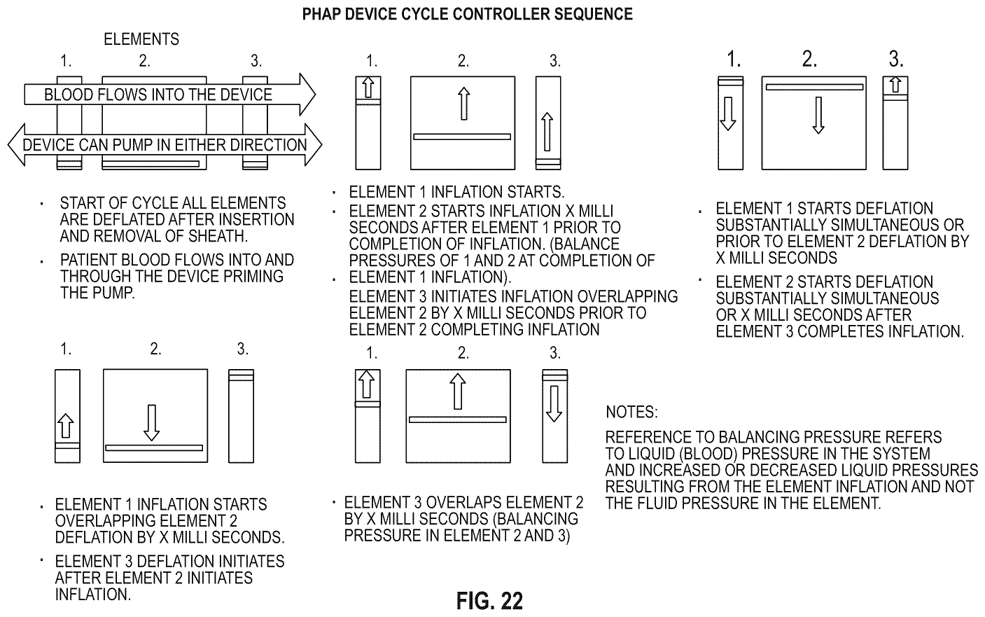

In some embodiments, the system comprises one or more computers comprising one or more processors and one or more non-transitory computer readable media. In some embodiments, the one or more non-transitory computer readable media include instructions stored thereon that cause the one or more computers to implement one or more programming steps by the one or more processors. FIG. 22 depicts steps implemented by one or more configurations described herein according to some embodiments, and also describe steps for a method of use.

Some embodiments include a step of executing an inflation sequence. In some embodiments, the inflation sequence includes a sequence for inflating two or more elements. Some embodiments include a step of executing a deflation sequence. In some embodiments, the deflation sequence includes a sequence for deflating two or more elements. In some embodiments, the inflation sequence for an element is different than a deflation sequence for a elements. Some embodiments include a step of executing an independent inflation and/or deflation command to a single element. Some embodiments include a step of executing multiple single elements commands to different elements in a pre-determined pattern.

Some embodiments include a step of executing an automatic and/or a manual mode. In some embodiments, a manual mode includes instructions to generate a graphical user interface (GUI) configured to enable an operator to manually set and/or change one or more system settings (e.g., desired resulting pressure). In some embodiments, one or more system settings includes element fluid and or heart assist pump speed and/or blood pressure set points, each of which the controller is configured to alter according to the system settings. In some embodiments, an automatic mode includes instructions executed by the one or more processors to maintain a blood pressure value. In some embodiments, the automatic mode includes instructions to automatically change its cycle rate (e.g., increase/decrease) to maintain a blood pressure value as conditions within a patient change.

In some embodiments, the heart assist pump 106 includes one or more sensors 312. In some embodiments, the one or more sensors 312 are located in the one or more of the proximal and distal ends of the heart assist pump 106. In some embodiments, the one or more sensors 312 are configured to monitor the blood pressure in the lower ventricle (LV) and/or (ascending) aorta. In some embodiments, the instructions cause the computer to receive input from the one or more sensors to implement one or more controls (e.g., when in automatic mode).

In some embodiments, the system includes a display (e.g., color touch screen) configured to display the GUI 101. In some embodiments, the GUI 101 comprises one or more control functions for the system. In some embodiments, the GUI 101 comprises a blood pressure reading in PSI and/or mmHg.

In some embodiments, the heart assist pump 106 includes a communication device 130. In some embodiments, the communication device 130 is configured to send one or more electronic transmissions to one or more controllers 102. In some embodiments, the communication device 130 is configured to send one or more electronic transmissions from the implant site and/or an area proximate the one or more components inside a patient. In some embodiments, an electronic transmission comprises a wireless signal. In some embodiments, the controller 102 comprises a receiver configured to receive the (wireless) electronic transmission. In some embodiments, an electronic transmission comprises data from one or more sensors 312. In some embodiments, an electronic transmission comprises a pump identification. In some embodiments, the pump identification is configured to verify the authenticity of the peristalsis heart assist pump 106.

In some embodiments, the heart assist pump 106 include a communication device 130. In some embodiments, the communication device 130 includes a pump identification device. In some embodiments, a pump identification device is configured to send patient data including one or more patient identification and/or medical details about the patient, the peristalsis heart assist pump, medical history, and/or any conventional information stored within the communication device 130. In some embodiments, the communication device 130 is configured to receive data through an electronic transmission. In some embodiments, the communication device 130 is configured to update, store, and/or replace data stored on one or more communication device non-transitory computer readable media with the received data. In some embodiments, the communication device 130 comprises one or more of a radio frequency identification (RFID) device, a Bluetooth® low energy (BLE) device, a near field communication device (NFC), and and/or an ultra-wide band (UWB) device, as a non-limiting examples.

In some embodiments, the heart assist pump 106 includes one or more check valves. In some embodiments, the one or more check valves are used in place of one or more balloons and or inlet and/or outlet check elements 1901. In some embodiments, one or more check valves include a collapsing valve configured to collapse against a wall and/or mandrel to allow and/or stop flow. In some embodiments, a collapsing valve configuration includes one or more of a modified umbrella, (multi-segmented) duck bill (e.g., FIG. 19), and other flexible material valve configuration. In some embodiments, a check valve includes an inflatable element. In some embodiments, at least a portion of an inflatable element is configured to function as a check valve, where the at least a portion is configured to inflate to substantially block the inlet of the housing before the rest of the element fully inflates forcing the fluid away from the inlet of the housing.

In some embodiments, the peristalsis heart assist pump 106 comprises one or more electromagnetic valves. In some embodiments, the electromagnetic valves are configured to control inlet and/or outlet flow through the peristalsis heart assist pump. In some embodiments, the electromagnetic valves are configured to operate in conjunction with the one or more check elements 1901. In some embodiments, the heart assist pump 106 is configured to pump fluid using the element fluid force and/or use electromagnetic elements 1905, 1906, 1907 to provide inlet and outlet check valve functions. In some embodiments, the heart assist pump 106 comprises one or more magnets 1905, 1906, 1907 configured to attract to one or more other magnets and/or ferromagnetic materials positioned in the area of the heart assist pump 106 inlet and/or outlet. In some embodiments, the mandrel 2007 and/or wall 2006 comprise one or more magnets 1905, 1907 configured to attract and/or repel one or more magnets 1906 and/or ferromagnetic materials 1906 coupled to one or more check valve elements 1901. In some embodiments, the one or more magnets 1905, 1907 include electromagnets configured to generate a magnetic field upon receiving an applied electrical current. In some embodiments, the electrical current is supplied and/or controlled by the one or more controllers 102. In some embodiments, an electromagnet 1905, 1907 is configured to repel the one or more magnets 1906 on the check element 1901 to open the check element 1901 (e.g., during a reverse flow). In some embodiments, an electromagnet 1905, 1907 is configured to attract the one or more magnets 1906 on the check element 1901 to open the check element 1901. Any element and/or check valve described herein may include magnets according to some embodiments.

In some embodiments, the pumping element 2001, 2002, 2003 comprises one or more pump magnets 2014 and/or ferromagnetic materials 2014 configured to control pumping action through heart assist pump 106. In some embodiments, the mandrel 2007 comprises one or more mandrel electromagnets 2016 configured to attract one or more magnets 2014 and/or ferromagnetic material 2014 integral and/or coupled to one or more pumping elements 2001, 2002, 2003. In some embodiments, the one or more mandrel magnets 2016 and/or one or more wall magnets 2015 are configured to enable the one or more pumping elements 2001, 2002, 2003 to directionally expand in the direction of the magnetic attraction. In some embodiments, the controller is configured to initiate one or more magnetic actuation sequences similar to the pneumatic sequences described herein to create directional fluid flow. In some embodiments, the one or more controllers 102 are configured to initiate one or more magnetic attraction and/or repulsion sequences in conjunction with one or more fluid (e.g., pneumatic) actuation sequences.

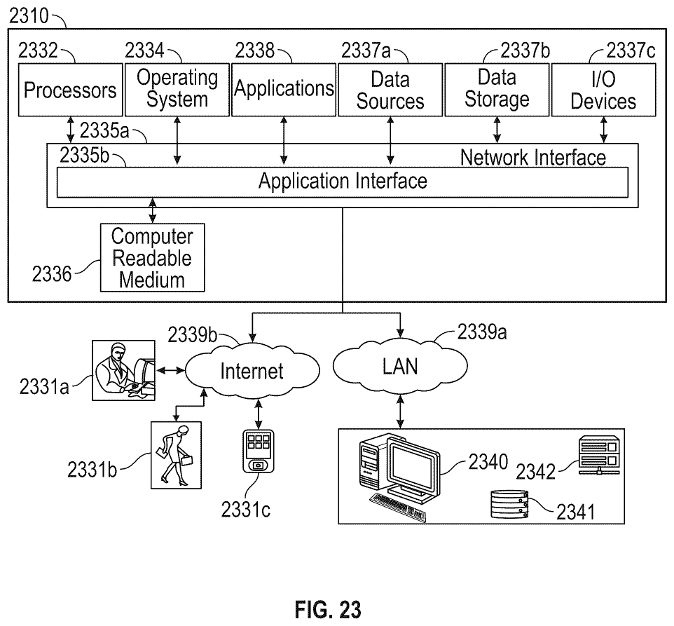

FIG. 23 illustrates a computer system 2310 enabling or comprising the systems and methods in accordance with some embodiments. In some embodiments, the computer system 2310 is configured to operate and/or process computer-executable code of one or more software modules of the aforementioned system and method. Further, in some embodiments, the computer system 2310 is configured to operate and/or display information within one or more graphical user interfaces (e.g., HMIs) integrated with or coupled to the system.

In some embodiments, the computer system 2310 comprises one or more processors 2332. In some embodiments, at least one processor 2332 resides in, or is coupled to, one or more servers. In some embodiments, the computer system 2310 includes a network interface 2335a and an application interface 2335b coupled to the at least one processor 2332 capable of processing at least one operating system 2334. Further, in some embodiments, the interfaces 2335a, 2335b coupled to at least one processor 2332 are configured to process one or more of the software modules (e.g., such as enterprise applications 2338). In some embodiments, the software application modules 2338 include server-based software. In some embodiments, the software application modules 2338 are configured to host at least one user account and/or at least one client account, and/or configured to operate to transfer data between one or more of these accounts using one or more processors 2332. Further program instructions are described below in relation to FIGS. 29-58.

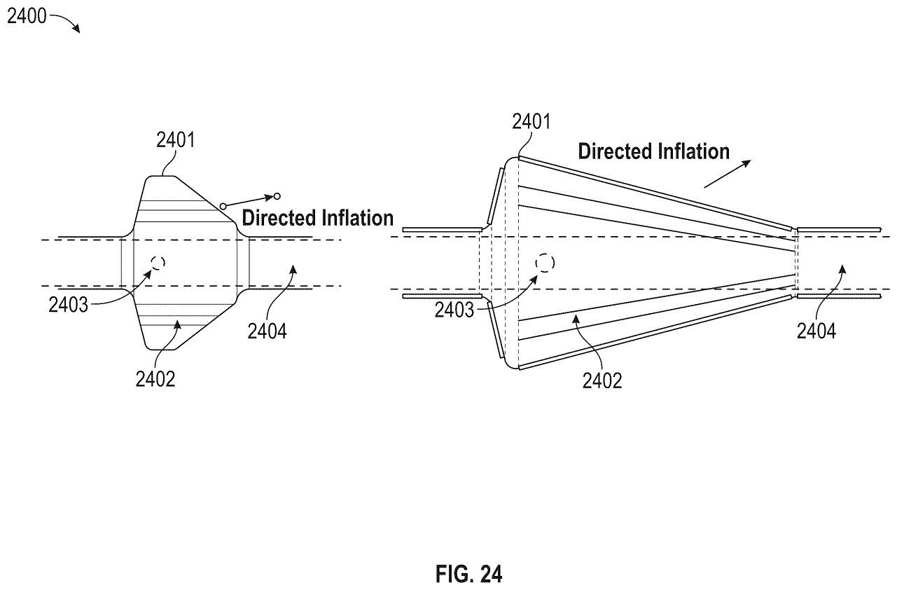

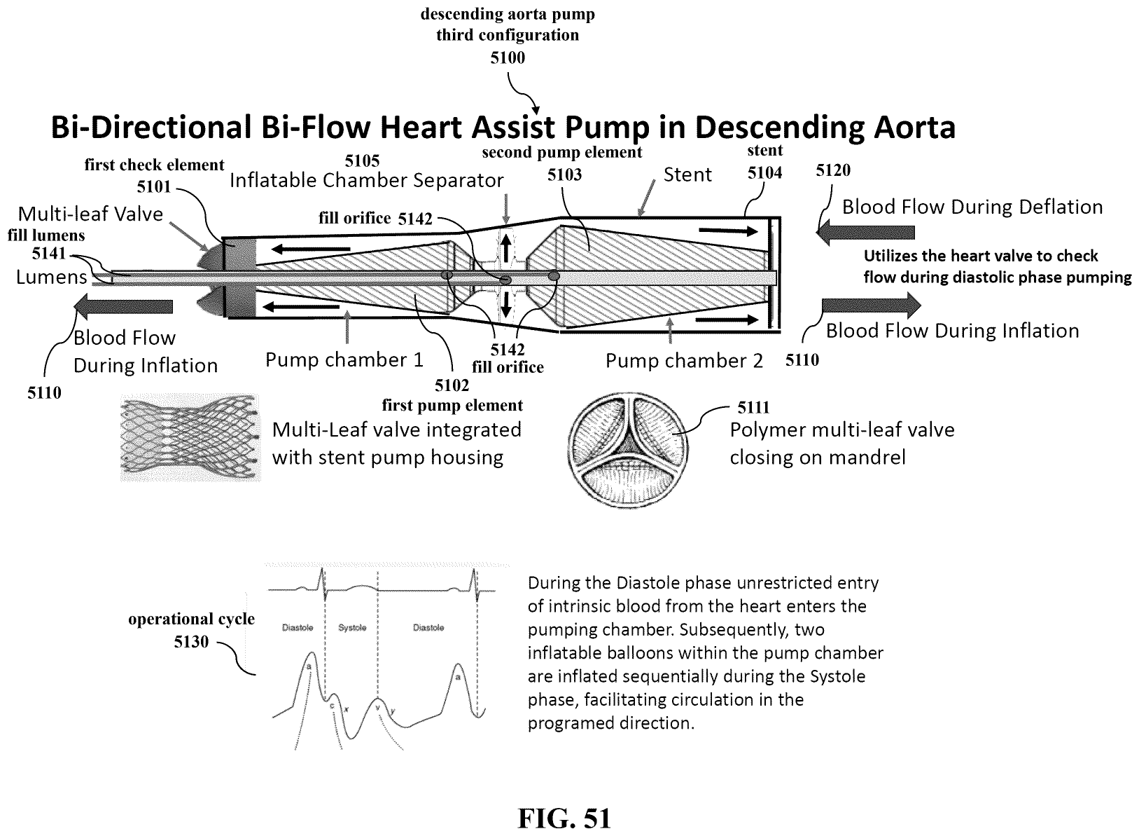

In some embodiments, the system comprises one or more pleated check and/or pumping elements. FIG. 24 shows a pleated element arrangement 2400 in a partially inflated configuration, according to some embodiments, suitable for directional pumping, such as in the non-limiting arrangement of FIGS. 50 and 51. In some embodiments, one or more pleated elements 2401 include one or more pleated balloons 2401. In some embodiments, one or more pleats 2402 are configured to cause a pleated element to lay down on a “filler mandrel” in an even and/or organized way 2501. This allows for a smaller diameter balloon when deflated according to some embodiments. In some embodiments, the pleated element comprises one or more fill ports 2403 which is configured to receive element fluid from a mandrel 2404 as previously described. In some embodiments, the system includes one or more balloons comprising varying wall thickness and/or densities. In some embodiments, the one or more pleats 2402 comprise one or more ribs including one or more rib configurations as shown in FIG. 24. In some embodiments, the one or more ribs are configured to create varying resistance across the one or more balloons. In some embodiments, the varying resistance is configured to force a desired inflation direction. In some embodiments, the varying resistance is a result of the varying density in balloon wall thickness.

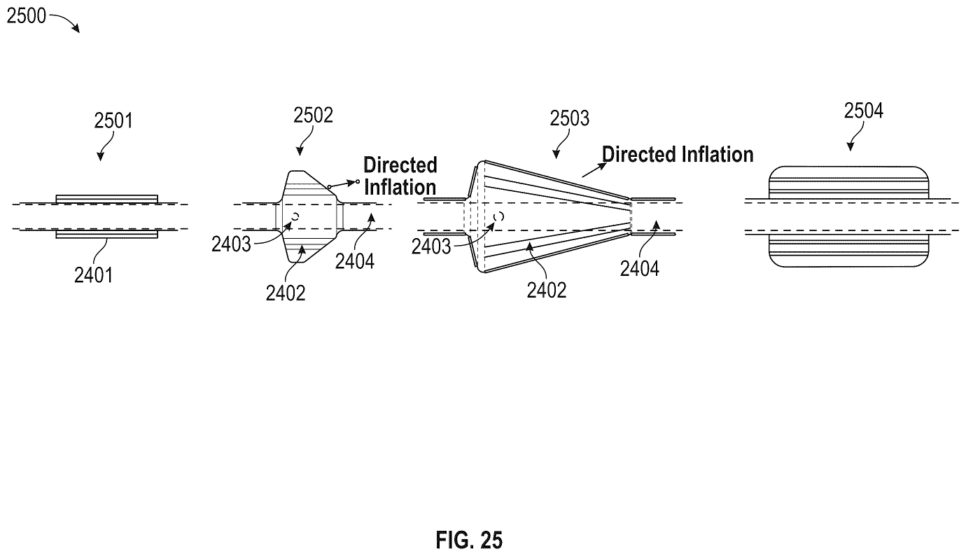

FIG. 25 illustrates the directional inflation 2500 of a pleated element according to some embodiments. In some embodiments, in a deflated state, the pleated element 2401 is configured to lay flat against the mandrel 2404. In some embodiments, upon receiving an initial element fluid through fill port 2403 the pleated element is configured to inflate to the shape 2502 which forces liquid forward. In some embodiments, shape 2502 acts to seal the housing inlet in a check valve manner as previously described. In some embodiments, as element fluid continues to pressurize the pleated element 2401 the pleated element 2401 continues to expand in the inflation direction 2503. In some embodiments, once a maximum pressure is achieved the pleated element is fully expanded 2504 inside a tube and/or stent as previously described.

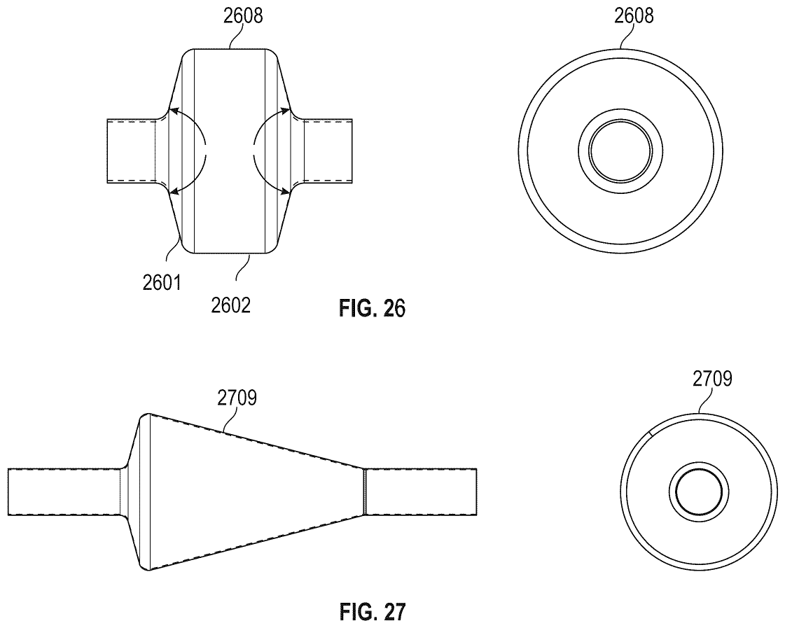

FIG. 26 shows a non-limiting example inlet check element 2608 in an inflated configuration according to some embodiments. In some embodiments, one or more outer portions 2601 of the check element 2608 comprise a higher density than one or more inner portions 2602. In some embodiments, while exact dimensions may vary, empirical data has shown that a minimum wall thickness of approximately 0.0007 inches is acceptable to maintain structural integrity in one or more elements described herein.

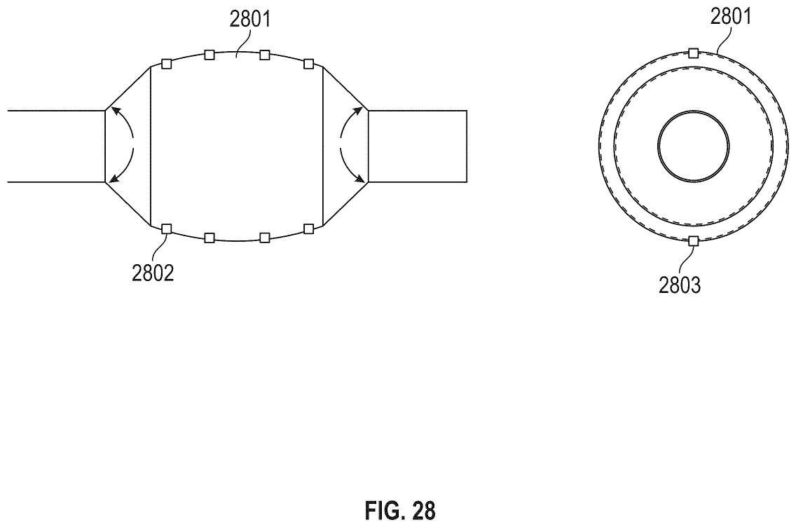

FIG. 27 shows a non-limiting example side and font view for a check element 2708 and a directional pumping element 2709 according to some embodiments. FIG. 28 shows a non-limiting example inflated bi-directional pumping element 2801 according to some embodiments. In some embodiments, one or more bi-directional pumping elements 2801 comprises one or more magnetic and/or magnet material 2802 configured to aid in shaping the bi-directional pumping element inflation and/or deflation as previously described. In some embodiments, one or more bi-directional pumping elements 2801 comprises one or more fluid ports 2803 configured to apply vacuum and/or pressure to aid in shaping the bi-directional pumping element inflation and/or deflation as previously described.

FIG. 29 shows an hour-glass configuration for the heart assist pump 106 according to some embodiments. In some embodiments, the one or more of the pumping elements described herein are configured to send fluid through a combination pump 2900 which is in the shape of an hour-glass in this non-limiting example. In some embodiments, the combination pump 2900 includes both an aortic pump 2901 and a left ventricle (LV) pump 2902. In some embodiments, the aortic pump 2901 is configured to pump fluid from the ascending aorta. In some embodiments, the LV pump is configured to pump fluid from the left ventricle.

In some embodiments, the stent 107 includes an hour-glass shaped stent 2917. In some embodiments, the hour-glass shaped stent 2917 includes an aorta pump section 2911 and an LV pump section 2912. In some embodiments, the aorta pump section 2911 and the LV pump section 2912 are separated by a reduced cross-section 2913. In some embodiments, the reduced cross-section 2913 is configured to cause the heart assist pump 106 to seat in the aortic valve, where in a deployed configuration the aorta pump section 2901 and the LV pump section 2902 are at least partially held in position by the force of the aortic valve against the reduced cross-section 2913. In some embodiments, the reduced cross-section 2913 is configured to be positioned in the aortic valve. In some embodiments, the reduced cross-section 2913 enables aorta pump section 2911 and LV pump section 2912 to both be larger than the reduced cross-section 2913, allowing larger flowrates than a single diameter pumping arrangement which is limited by the size of the aortic valve. In some embodiments, the reduced cross-section 2913 also enables isolation of the aortic valve to enable desired pumping characteristics.

In some embodiments, the aorta pump section 2911, the LV pump section 2912, and the reduced cross-section 2913 include a continuous material. In some embodiments, the continuous material includes an element housing 2904. In some embodiments, the element housing 2904 includes a tubular stent comprising a fluid impermeable wall configured to prevent the passage of fluid, enabling the pumping action described herein according to some embodiments. In some embodiments, the impermeable wall is integral with the stent 2917. In some embodiments, the impermeable wall is coupled to the stent 2917.

In some embodiments, the combination pump 2900 includes an LV check valve 2905, an LV pumping element 2906, a cross-section element 2907, an aorta pumping element 2908, and/or an aorta check valve 2909. As shown in further examples, one or more pumping elements 2908, 2906 may comprise a check valve 2905, 2909, respectively, according to some embodiments. In some embodiments, cross-section element 2907 may comprise any pumping element and/or check valve described herein, such as check element 2008, for example. In some embodiments, the reduced cross-section 2913 comprises no pumping element or check valve/element. Any reference to a “valve” as used herein can be replaced with “element” when defining the metes and bounds of the system, as a pumping element can also be configured as a check valve according to some embodiments. In some embodiments, the reduced cross-section 2913 is configured to expand the aortic valve to a diameter less than a diameter of the LV pumping element 2906 and/or the aorta pumping element 2908. In some embodiments, the stent 2917 reduced cross-section 2913 is configured to hold the aortic valve in an open position. In some embodiments, the aortic check valve 2909 and/or cross-section element 2907 is configured to prevent a backflow of fluid into the left ventricle while the aortic valve is held open by at least a portion of the stent 2917.

In some embodiments, one or more of the element housing 2904, LV check valve 2905, the LV pumping element 2906, the cross-section element 2907, the aorta pumping element 2908, the aorta check valve 2909, and/or the stent 2917 is couple to a mandrel 2921. In some embodiments, mandrel 2921 is similar in construction and/or function to mandrel 2404 and/or mandrel 2007, as non-limiting examples.

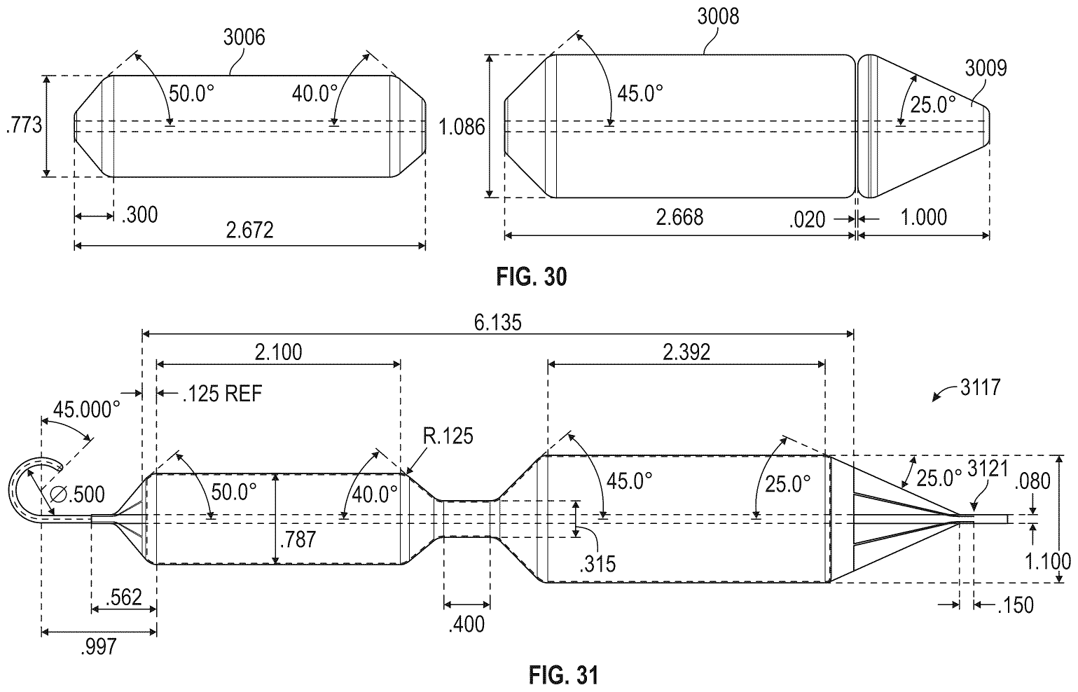

FIG. 30 shows non-limiting example dimensions for the LV pumping element 3006, the aorta pumping element 3008, and the aorta check valve 3009 of FIGS. 32-40, although any element or check valve describe herein can be used in accordance with some embodiments. FIG. 31 shows non-limiting example dimensions for the stent 3117 and mandrel 3121 of FIGS. 32-40 in inches according to some embodiments. In some embodiments, a range for a major diameter of one or more portions of a heart assist pump is between 0.197 in and 1.97 in (5 mm and 50 mm).

FIG. 32 shows a non-limiting heart assist pump 106 in a deployment configuration according to some embodiments. In some embodiments, the heart assist pump 106 is similar in configuration to the heart assist pump 2900. As shown in FIG. 32, the LV pumping element 2906 (element #1; first element), the aorta pumping element 2908 (element #2; second pumping element), and the aorta check valve 2909 (element #3; third element) are inflated in a first deployed configuration. In some embodiments, each element 2906, 2908, 2909 are similar in construction to FIGS. 24 and 25, where a portion of each element is configured to act as a check valve when partially inflated, limiting the number of elements, valves, and lumens needed for operation.

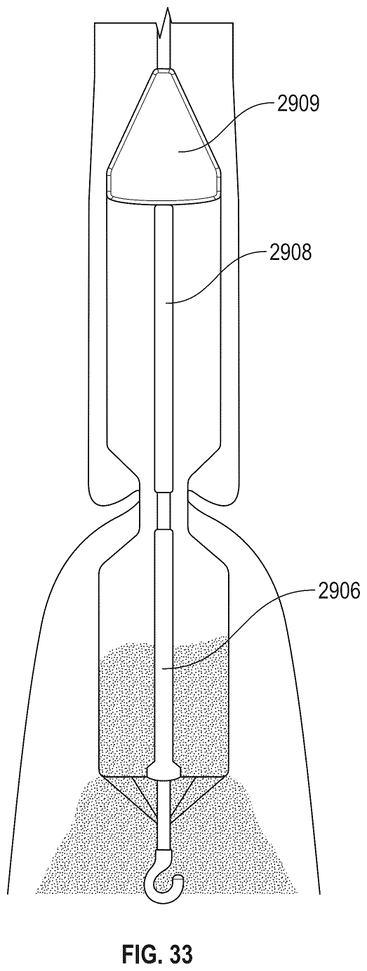

FIG. 33 depicts a flow chamber filling cycle according to some embodiments. In some embodiments, the controller 102 is configured to deflate the LV pumping element 2906 allowing the LV flow chamber in the LV pump section 2902 to fill with fluid. Simultaneously, (e.g., less than 1 second), the aorta pumping element 2908 is deflated in aorta pump section 2901, while the aorta check valve 2909 remains inflated, creating a low pressure zone in each section.

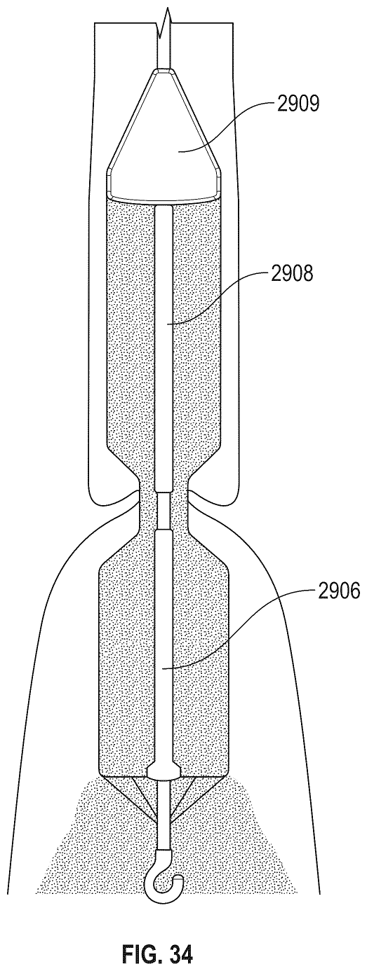

FIG. 34 illustrates a flow chamber sealing cycle according to some embodiments. One both sections 2901, 2902 are filled with fluid, the controller 102 is configured to keep the aorta check valve 2909 inflated and the aorta pumping element 2909 deflated as element fluid (e.g., air) is supplied the LV pumping element 2906 causing the element 2906 to partially inflate, blocking the inlet to the heart assist pump 2900.

FIG. 35 shows a left ventricle flow chamber pump cycle according to some embodiments. As shown in FIG. 35, the LV pumping element 2906 is configured to seal a fluid inlet and/or fluid outlet when partially inflated as previously described according to some embodiments. In some embodiments, this partial inflation acts as the check valve 2905 within the combination pump 2900. Once the LV pumping element 2906 has sealed the inlet, the aorta check valve 2909 deflates. At this time, in some embodiments, the LV pumping element 2906 continues to inflate forcing fluid from the LV pump section 2902 through the reduced cross-section 2913, into the aorta pump section 2901, and through the heart assist pump 106 outlet.

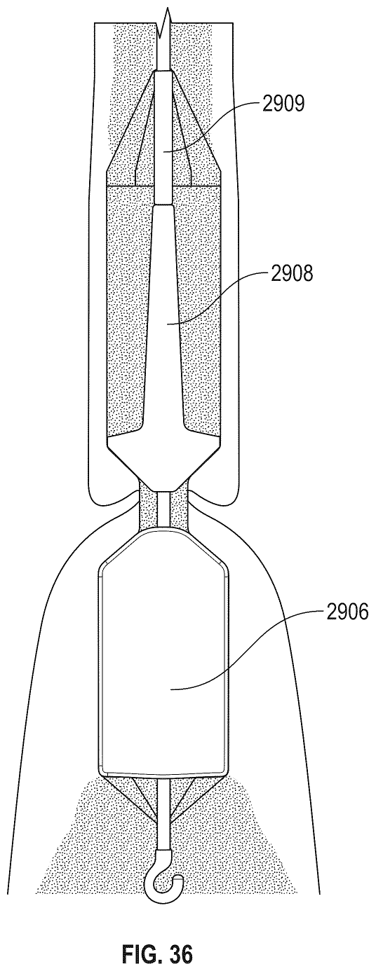

FIG. 36 depicts an aortic flow chamber according to some embodiment. In some embodiments, once LV pumping element 2906 is fully inflated, the aorta pumping element partially inflates blocking and/or sealing the reduced cross-section 2913, acting similar to check element 2907, which is not included in this non-limiting embodiment to demonstrate the range of functions the different elements/valves described herein can be adapted to perform.

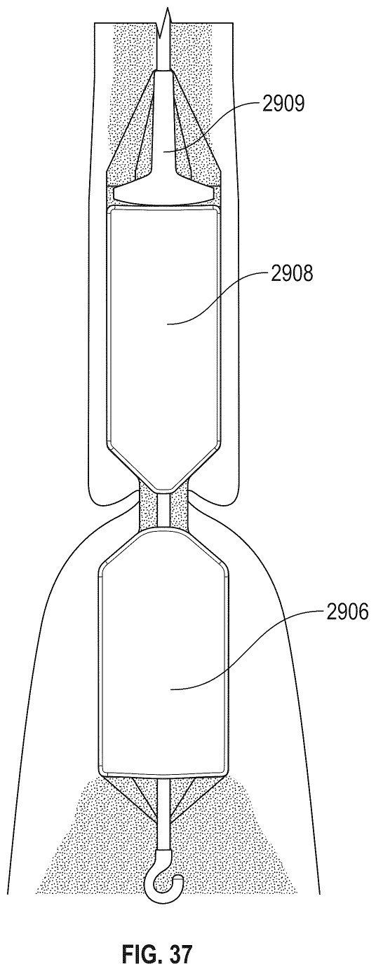

FIG. 37 illustrates an aortic seal and pump cycle according to some embodiments. In some embodiments, once aorta pumping element 2908 is fully inflated, aorta check valve 2909 partially inflates, sealing the outlet of the heart assist pump 106.

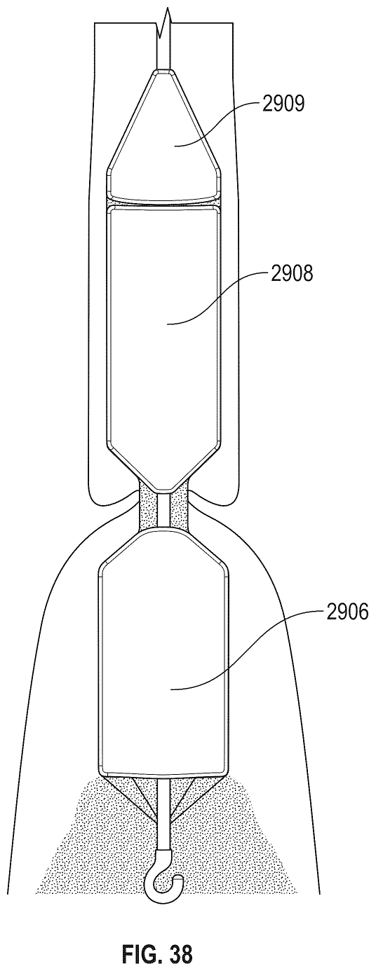

FIG. 38 shows a completed pump cycle according to some embodiments. In some embodiments, the aorta check valve 2909 continues to inflate, directional forcing remaining fluid from the combination pump 2900.

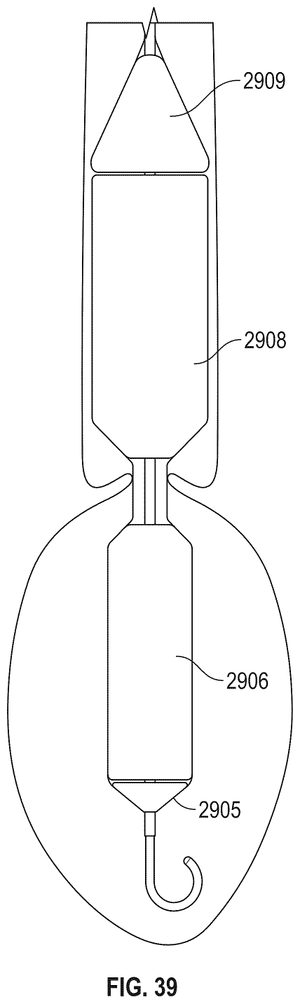

FIG. 39 shows another configuration of heart assist pump 2900 with an additional check valve 2905 included, where the check valve 2905 may include elements 2708, 2709, or any other element or check valve described herein. The functionality is the same as FIGS. 32-38 and will not be repeated in the interest of being concise.

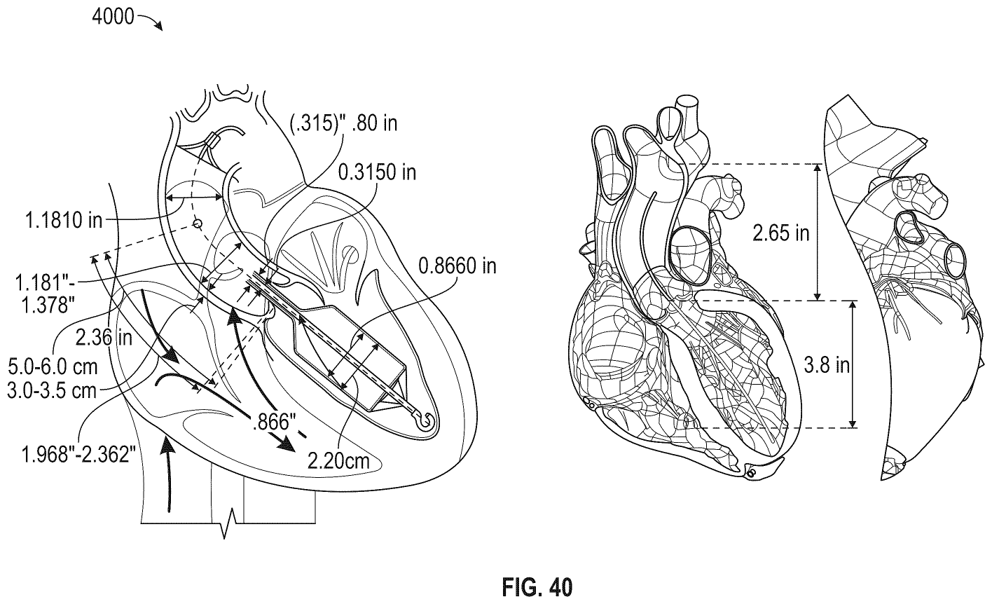

FIG. 40 illustrates a drawing of the system within a heart 4000 according to some embodiments. In some embodiments, the aorta pump section 2901 may extend between 1.5 and 3 inches (e.g., approximately 2.65 inches) into the ascending aorta as measured from the aortic valve. In some embodiments, the LV pump section 2902 may extend between 2 and 4 inches (e.g., approximately 3.8 inches) into the left ventricle as measured from the aortic valve. These distances will vary according to element and valve configurations according to some embodiments and are not to be construed as limiting.

The following description depicts steps for extracting the heart assist pump 106 when a procedure is complete according to some embodiments. Some embodiments include a step of moving an outer sheath 2922 back down the catheter and/or mandrel 2921. Some embodiments include a step of compressing, recapturing, and/or drawing the heart assist pump 106 back into the sheath 2922. Some embodiments include a step of pulling back the catheter with the heart assist pump 106 inside through an incision in the femoral artery. In some embodiments, once removed from the patient, the heart assist pump 106 and/or catheter/mandrel is configured to be disposed.

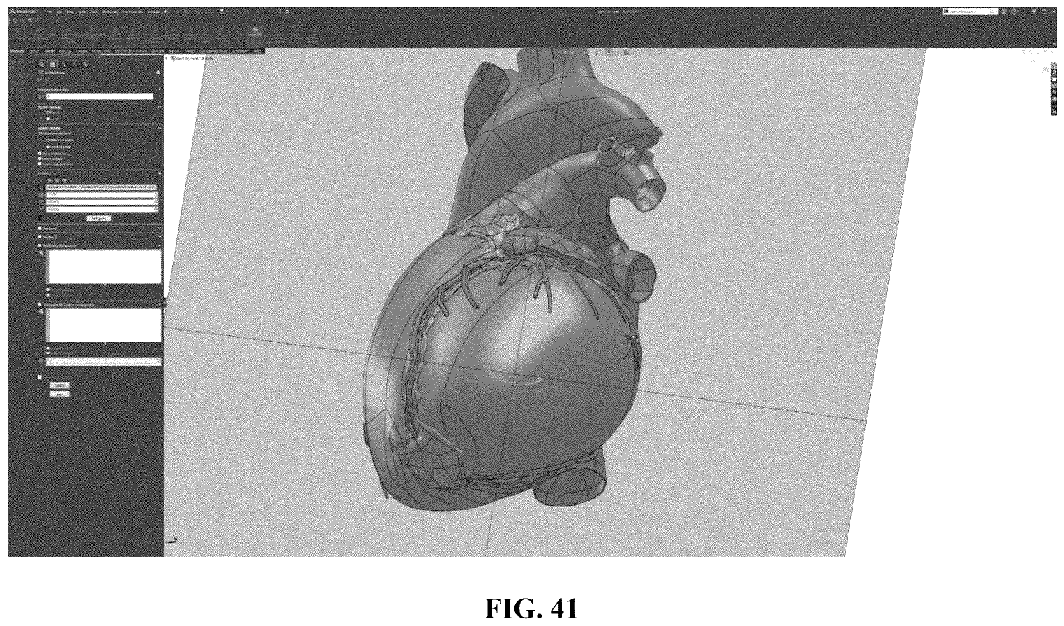

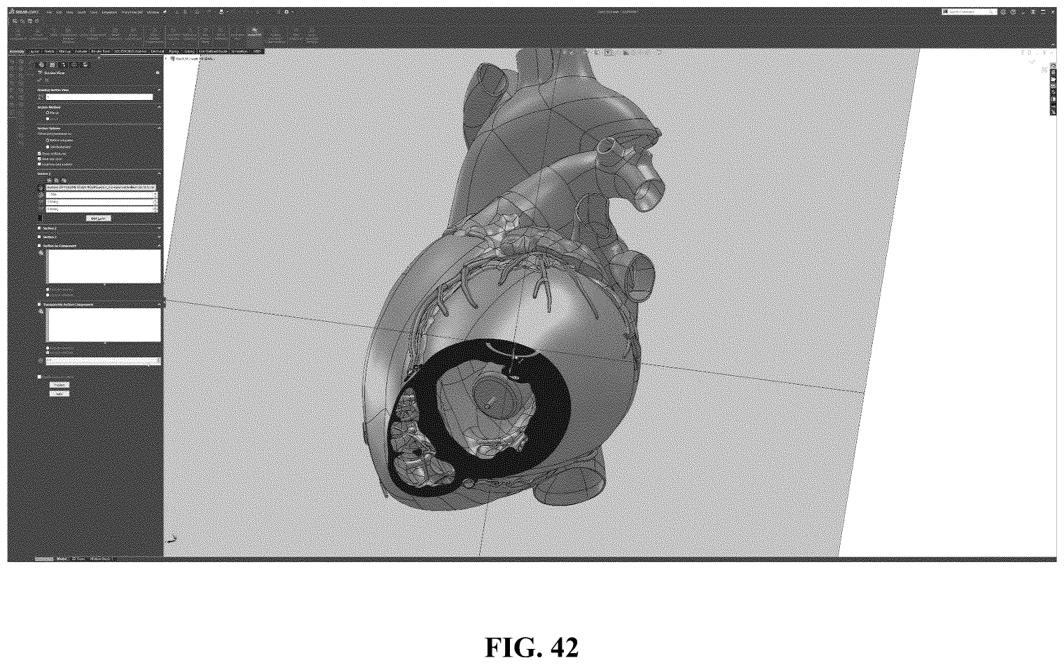

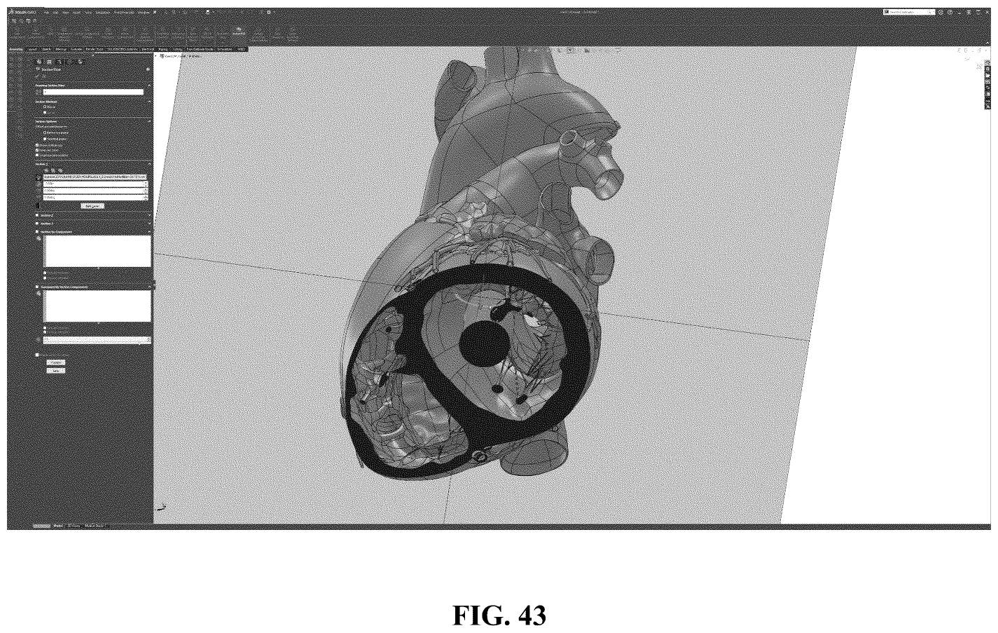

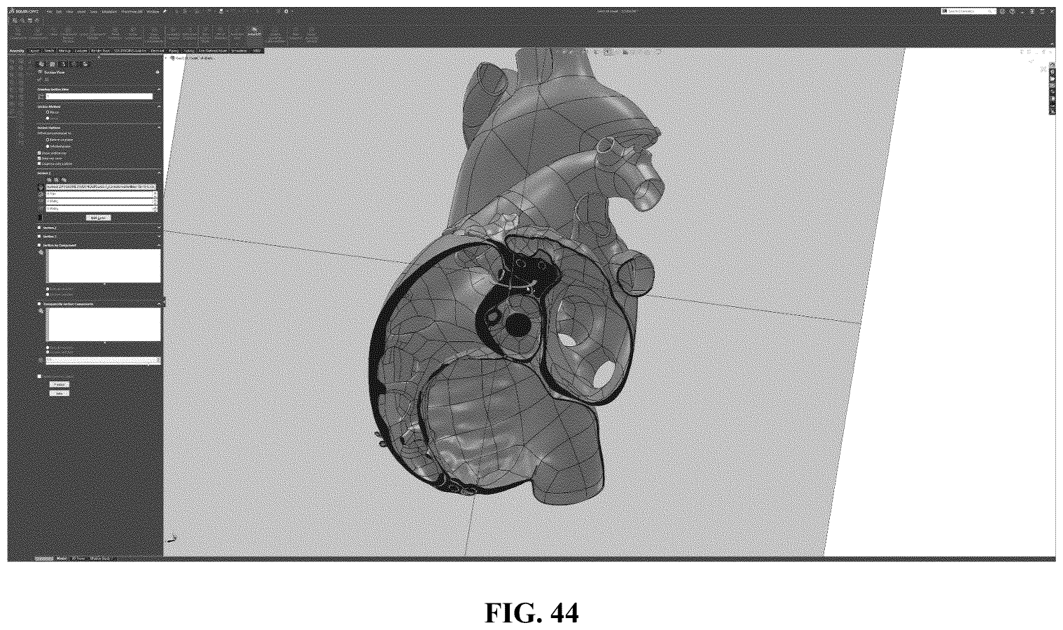

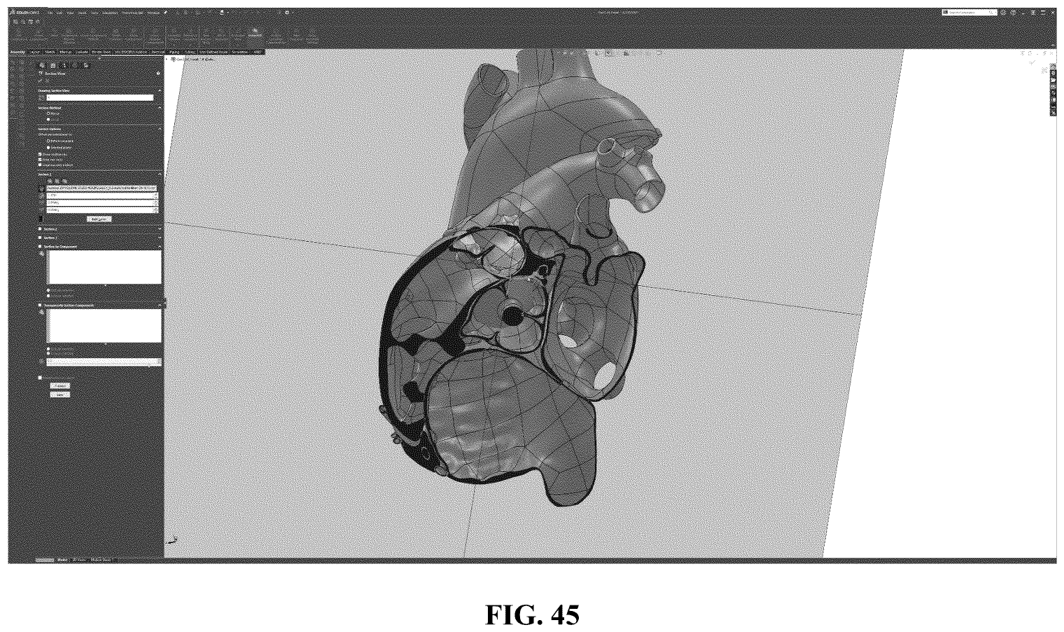

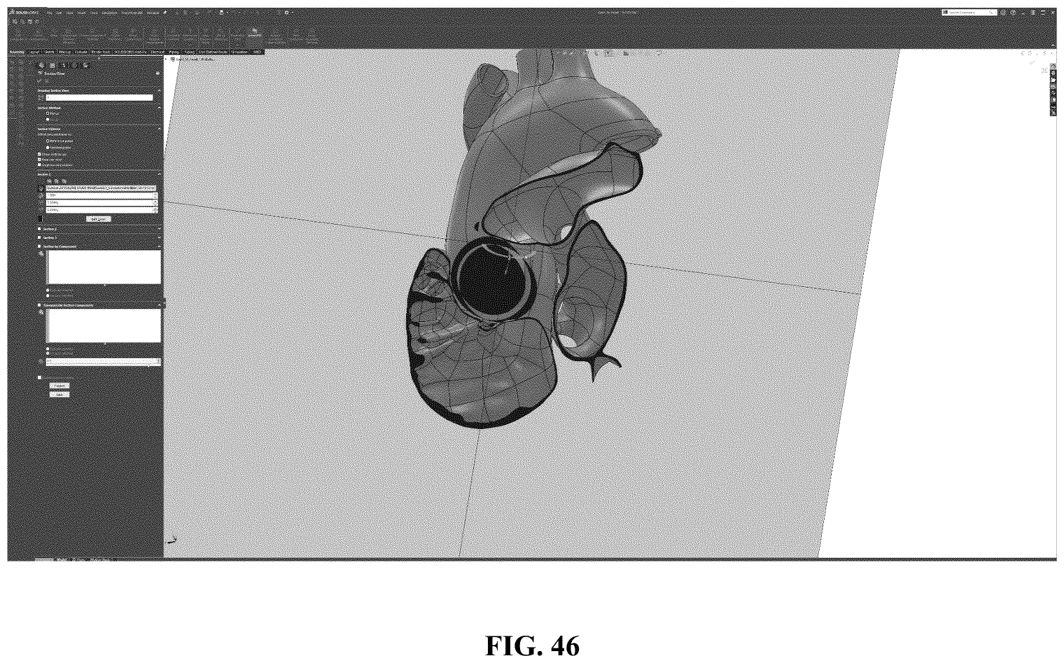

FIG. 41 shows a first rendered sectional view of an installed system according to some embodiments. FIG. 42 shows a second rendered sectional view of the installed system according to some embodiments. FIG. 43 shows a third rendered sectional view of the installed system according to some embodiments. FIG. 44 shows a fourth rendered sectional view of the installed system according to some embodiments. FIG. 45 shows a fifth rendered sectional view of the installed system according to some embodiments. FIG. 46 shows a sixth rendered sectional view of the installed system according to some embodiments.

FIG. 47 illustrates the system deployed as a descending aorta pump, in accordance with some embodiments. In some embodiments, the system described herein and/or one or more (e.g., any) combinations of element described herein and/or presented in any figures are configured to be deployed as a descending aorta pump. While FIG. 40 illustrates the system in a configuration that passes through the aortic valve and occupies a volume extending into the left ventricle, deployment within the descending aorta, as shown in FIG. 47, in accordance with some embodiments, provides several structural and physiological benefits.

As a non-limiting example, when the system is positioned entirely within the descending aorta, the system can use the aortic valve as a natural check valve, preventing retrograde flow into the heart during systole. In this configuration, the descending pump is configured to generate a reverse-directed flow that moves blood proximally toward the aortic arch during diastole, which may advantageously perfuse the upper extremities and cerebral vasculature. Because the aortic valve remains closed during diastole, the pump-generated proximal flow is redirected through the innominate, left carotid, and left subclavian arteries. In this manner, a bidirectional descending aorta pump positioned in the descending aorta enables controlled redirection of blood toward the upper body during a phase of the cardiac cycle when forward cardiac output is low or absent. The configuration shown in FIG. 47 eliminates the need for valve crossings, avoids mechanical interaction with the left ventricular outflow tract, and allows for isolated endovascular deployment without interference with the native valve function.

While various configurations and combinations of elements described herein i assist in pumping blood from the heart, in some embodiments, the system is configured to assist in delivering blood to the coronary arteries which feed the heart itself the nutrients it needs. In some embodiments, the system is configured to aid blood flow to the coronary arteries when the heart relaxes after a contraction.

During diastole, the aortic valve is closed and the myocardium is in a relaxed state, creating a natural opportunity for blood to flow into the coronary arteries, which originate near the aortic root just above the valve. In some embodiments, the system is configured to generate a controlled flow of blood in the retrograde direction from the descending aorta toward the aortic arch during diastole. This retrograde flow is timed by the controller 102 so that the blood flow is redirected by the closed aortic valve away from the left ventricle and instead directed into the coronary ostia, thereby enhancing perfusion of the coronary arteries. The aortic valve functions as a unidirectional barrier that prevents backward flow into the ventricle and creates a fluidic seal against which the system may deliver retrograde pressure. This action directs blood flow into the aortic arch and coronary vasculature, facilitating increased myocardial oxygen delivery during the diastolic phase without altering ventricular hemodynamics.

In some embodiments, the retrograde flow enters the branching vessels of the aortic arch, including the brachiocephalic (innominate) artery, left common carotid artery, and left subclavian artery. As a result, in some embodiments, the descending aorta pump is configured to augment perfusion to the upper extremities and cerebral circulation during diastole, when forward flow from the heart is minimal. The ability to direct pressurized flow proximally during the relaxation phase enables the system to enhance end-organ perfusion in a manner synchronized with the native cardiac cycle.

In some embodiments, by delivering blood toward the aortic root without physically crossing the valve, the system preserves the native valve's functional integrity while achieving bidirectional pumping. This configuration may be advantageous in minimizing the risk of hemolysis, thrombus formation, and interference with the ventricular outflow tract, all while supporting diastolic augmentation to both the coronary and arch-supplied circulations.

The pump shown in FIG. 40 includes a neck region 2907 positioned between two pumping chambers, in accordance with some embodiments, each of which may include inflatable elements or structures configured to generate pressure differentials during operation. The neck region 2907 has a narrowed cross-sectional diameter relative to adjacent components 2906 and 2908, forming a waist-like geometry that facilitates passage through the aortic valve. The relatively narrow central neck 2907 allows the system to straddle the aortic annulus and extend into both the left ventricular outflow tract and the ascending aorta without impeding native valve motion, thereby enabling the system to function as an inline pump within the natural blood flow path exiting the heart.

While the configuration in FIG. 40 may be used as a descending aorta pump (i.e., be configured to be placed in the descending aorta, the pump shown in FIG. 47 does not include a central neck region and may instead comprise a continuous, tapered, or variably contoured body configured to reside entirely within the descending aorta. The absence of a constricted neck maintains uniform and/or gradually transitioning diameters along its longitudinal axis, thereby facilitating a conformal fit within the aortic lumen. In some embodiments, the external surface of the pump may be shaped, coated, or structured to create a seal against the interior wall of the descending aorta to direct flow and prevent leakage. In some embodiments, the pump may be radially expandable, compliant, or segmentally inflatable to accommodate variations in vessel diameter and curvature, enhancing positional stability and sealing performance without the need for valve crossing or anchoring across the aortic annulus. Further sealing methods using inflatable elements are discussed, infra.

In some embodiments, the system includes a catheter. Some embodiments include a step of inserting the system (e.g., a catheter with or without a stent) into the aorta. Some embodiments include a step of creating an opening in the leg and/or arm to insert the system. Some embodiments include a step of guiding the pump (e.g., plurality of pump elements) to the aorta. Some embodiments include a step of initiating program instructions that are configured to cause the plurality of balloons to inflate according to one or more descriptions herein when the heart relaxes. Some embodiments include a step of initiating program instructions that are configured to cause the plurality of balloons to deflate according to one or more descriptions herein when the heart contracts. In some embodiments, the system is configured to receive, by the one or more processors, sensor data (e.g., pressure) indicating heart state (e.g., contraction, relaxation) and/or control initiation of one or more of the plurality of pump elements according to sensor data. In some embodiments, descending aorta pump configurations can comprise a single element as illustrated in FIG. 3, two elements similar to FIG. 14, three pump elements as described in FIGS. 22 and/or 48, or more than three elements as shown in FIG. 16.

In some embodiments, the present system improves over prior art systems by enabling control of the fluid (e.g., blood) flow direction, increasing flowrate, providing a smaller catheter, providing smaller pumping elements, and/or enabling increased cycle speeds.

In some embodiments, the intra-aortic pump provides the benefit of fluid flow direction control. In some embodiments, program instructions included to alter, by the one or more processors, a fluid flow direction to and/or from the heart. In some embodiments, the GUI comprises one or more icons for changing a fluid flow direction.

In some embodiments, the system is configured to provide flow rates up to 8 liters per minute. In some embodiments, the system is configured to provide flow rates up to 6 liters per minute. In some embodiments, the system is configured to execute, by the one or more processors, cycling speeds of a range of 10-200 cycles per minute (CPM).

In some embodiments, one or more sensors include one or more electrocardiogram (EKG) electrodes. In some embodiments, the system is configured to execute, by the one or more processors, a pump initiation based on values received by one or more EKG electrodes. In some embodiments, one or more sensors include one or more pressure sensors. In some embodiments, the one or more pressure sensors are proximate the one or more pump elements. In some embodiments, the pressure sensors are proximate the one or more element fluid pumps 103. In some embodiments, the one or more pressure sensors are located between the one or more pump elements and the one or more element fluid pumps 103. In some embodiments, one or more pressure sensors are configured to detect pressure changes in one or more fill lumens of any of the configurations described herein. In some embodiments, changes in fill lumen pressure are the result of changes in pressure within the heart. In some embodiments, the system is configured to execute, by the one or more processors, a pump initiation based on values received by one or more pressure sensors.

In some embodiments, the controller is configured to receive, execute, and/or maintain one or more pump speeds. In some embodiments, one or more commands described herein are received via the GUI, where the GUI comprises one or more inputs for each of the one or more commands (e.g., setpoints, configurations, etc.) described herein. In some embodiments, one or more commands includes one or more pulsation commands. In some embodiments, one or more pulsation commands include instructions to execute, by the one or more processors, an inflation and/or deflation sequence, where the inflation and/or deflation sequence includes a timing of the element fluid pump 103 execution.

FIG. 47 illustrates the system deployed as an intra-aortic pump in inflated and deflated configurations, during diastole and systole, respectively, in accordance with some embodiments. As used herein, the term “diastole” refers to a relaxation phase of the cardiac cycle during which the heart muscle relaxes and allows the heart chambers to fill with blood. During diastole, the ventricles are in a relaxed state, and blood flows from the atria into the ventricles through the open atrioventricular valves, which include the mitral valve on the left side of the heart and the tricuspid valve on the right side of the heart. The semilunar valves, which include the aortic valve and pulmonary valve, are closed during diastole to prevent blood from flowing backward into the ventricles.

The term “systole” refers to a contraction phase of the cardiac cycle during which the heart muscle contracts and forces blood out of the heart chambers. During ventricular systole, the ventricles contract, causing the atrioventricular valves to close and the semilunar valves to open. Closure of the atrioventricular valves prevents blood from flowing back into the atria, while the opening of the semilunar valves allows blood to be ejected from the right ventricle into the pulmonary artery and from the left ventricle into the aorta. Systole results in the circulation of blood to the lungs for oxygenation and to the systemic circulation to deliver oxygenated blood to tissues throughout the body.

The cardiac cycle alternates between diastole and systole in a rhythmic manner to maintain continuous blood flow. Diastole occupies a longer duration of the cardiac cycle than systole under resting conditions, allowing sufficient time for ventricular filling. In some embodiments, the sensor measurement (e.g., sensor 312) during these phases enable the system to time the pump to the cardiac cycle. In some embodiments, pressure sensors associated with controller 102 are configured to sense changes in pressure through one or more fill lumens.

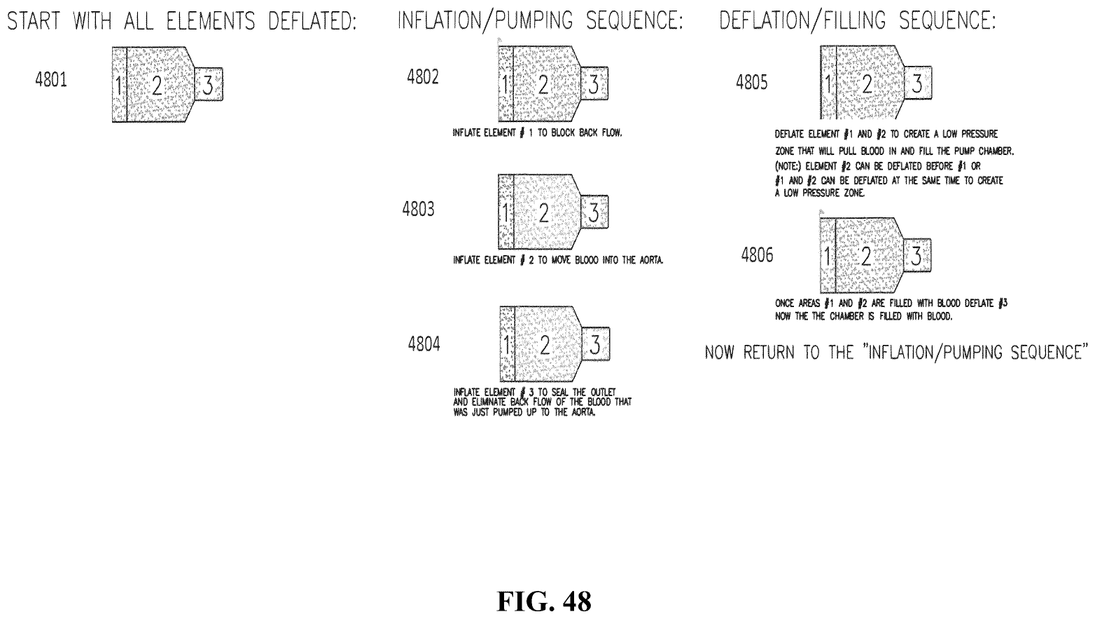

FIG. 48 depicts an inflation and deflation sequence and/or method for an intra-aortic pump actuation according to some embodiments. In some embodiments, the sequence begins at step 4801 with all elements deflated. Some embodiments include an inflation and/or pumping sequence step 4802 of inflating element 1 to block back flow. Some embodiments include a step 4803 of inflating element 2 to move blood into the aorta. Some embodiments include a step 4804 of inflating element 3 to seal the outlet and/or eliminate backflow of blood that was recently pumped up to the aorta. Some embodiments include a deflation and/or filling sequence that includes a step 4805 of deflating element 1 and 2 to create a low-pressure zone configured to pull blood in and help fill the pump chamber. In some embodiments, element 2 is deflated before element 1. In some embodiments, element 2 and element I are deflated at the same time. In some embodiments, once areas 1 and 2 are filled with blood, a step 4806 includes deflating element 3, thereby filling the entire chamber with blood and returning the system to the beginning for another cycle.

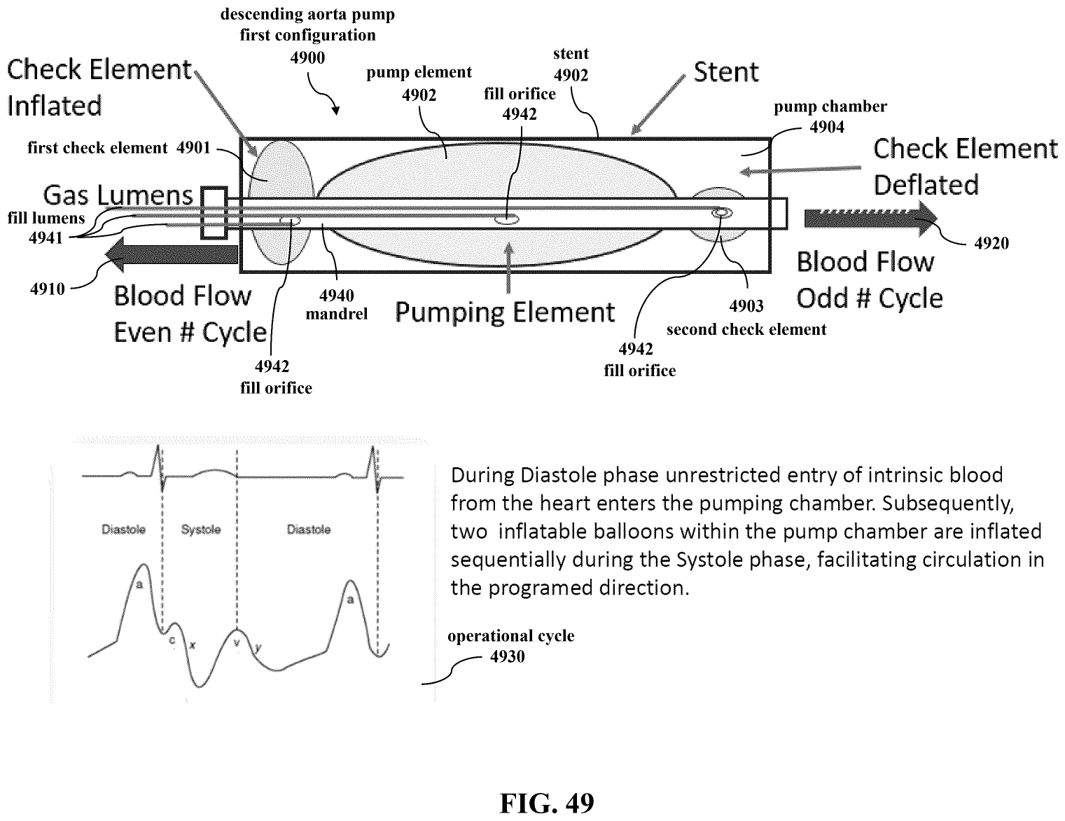

FIG. 49 illustrates a descending aorta pump first configuration 4900 for bidirectional blood flow control in the descending aorta, in accordance with some embodiments. The alternating directional counter pulsation pump configuration described in relation to FIG. 49, in accordance with some embodiments, solves a flow problem in unidirectional and balloon pumps situated in the descending aorta, where adequate blood flow to both upper and lower extremities may not be sufficiently maintained. In some embodiments, the system configuration illustrated in FIGS. 49-55 may include a combination of any of the structures (e.g., elements, lumens, check valves, balloons, stents) described herein to achieve the recited functionality to overcome the potential imbalance in blood perfusion.

In some embodiments, the system includes a flexible, expandable stent 4902 configured for aortic anchoring. In some embodiments, the stent houses a central inflatable pump chamber 4904 configured to enclose one or more inflatable elements including a pump element 4902, a first check element 4901 positioned proximally, and a second check element 4903 positioned distally. In some embodiments, the system is configured to execute a systematic cycle sequence of inflating check elements. In some embodiments, this sequence is configured to alternately direct blood flow in two distinct directions simultaneously.

In some embodiments, during the diastole phase, the system is configured to permit unrestricted entry of intrinsic blood from the heart into the pumping chamber 4904 through the proximal opening. In some embodiments, check element 4901 and pump element 4902 include inflatable balloons configured to inflate in a pre-programed sequence during the systole phase, which enables circulation in the direction programmed by the system's control logic. In some embodiments, this approach overcomes the limitations associated with unidirectional and balloon pumps in the descending aorta by maintaining desired blood flow to both upper and lower extremities.

In some embodiments, the alternating directional counter pulsation pump 4900 includes control logic configured to systematically sequence the inflation and deflation of two or more elements (e.g., 4901, 4902, 4903), enabling bidirectional blood flow. In some embodiments, the pump 4900 includes control logic to orchestrate the inflation and deflation of specific elements using a phased execution operational cycle 4930 synchronized with the cardiac cycle. In some embodiments, the system includes sensors or synchronization circuitry to align the cycle with intrinsic cardiac activity, as described supra.

In some embodiments, all inflatable elements are in a deflated state in the initial stage, allowing blood from the heart to enter the pumping chamber 4904 via the proximal end during diastole. In some embodiments, the first check element 4901 is selectively inflated to block retrograde entry, while the pump element 4902 inflates to direct flow forward during systole. In some embodiments, the mandrel 4940 provides structural support within the pump chamber 4904, and may incorporate fill lumens 4941 and fill orifices 4942 for delivering pressurizing gas to the inflatable elements. In some embodiments, fill lumens 4941 are integrated along the longitudinal axis of the mandrel 4940 to coordinate inflation pressures in the desired sequence.

In some embodiments, as the cycle progresses, the first check element 4901 and the pump element 4902 deflate, and the second check element 4903 inflates to prevent retrograde flow into the pump chamber 4904. In some embodiments, this alternating action propels blood in the forward (e.g., distal) direction during even-numbered cycles and reverses during odd-numbered cycles, as illustrated by directional flow indicators 4910 and 4920, respectively. In some embodiments, the second check element 4903 subsequently deflates, returning the system to the initial state, and initiating a subsequent or reversed operational cycle.

In some embodiments, the descending aorta pump first configuration 4900 is configured as an alternating directional counter pulsation pump to enable flexibility in directional flow. In some embodiments, the control logic includes program instructions that are configured to execute variations in the inflation sequence, wherein the instructions may cause one or more processors to execute a flow command to alternate flow direction with every cycle, and/or persist flow in a first direction for several cycles before reversing flow while the aortic valve is closed. This adaptability adds another layer of customization to the pump's operation, catering to individual patient needs and optimizing blood perfusion throughout the body. In some embodiments, the control system is configured to operate in a counter-pulsation mode timed to reduce interference with intrinsic heart function.

In some embodiments, the pump chamber 4904 and inflatable elements 4901, 4902, and 4903 are constructed from biocompatible polymer materials suitable for percutaneous delivery. In some embodiments, a helium or gas-based drive system is configured to sequentially actuate the inflatable elements via the gas lumens and fill lumens 4941 and fill orifices 4942. In some embodiments, the inflation sequence is programmable and adjustable based on patient hemodynamics, and may be dynamically modulated via control logic in response to input from sensors or user-defined clinical parameters. In some embodiments, the alternating directional counter pulsation configuration described herein effectively addresses the potential imbalance in blood perfusion associated with unidirectional pumps, ensuring adequate blood flow to both upper and lower extremities.

FIG. 50 illustrates descending aorta pump second configuration 5000 (a bi-directional opposed balloon heart assist pump in the descending aorta) configured for enhanced blood flow distribution, in accordance with some embodiments. In some embodiments, descending aorta pump second configuration 5000 includes structural and functional features derived from elements described in FIGS. 19-28, and other figures referenced throughout this disclosure. In some embodiments, pump elements 5002 and 5003 (e.g., inflatable balloons) are housed within stent 5004 configured for endoluminal anchoring within the descending aorta. In some embodiments, the pump includes first check element 5001 positioned at a proximal end and multi-leaf polymer valve 5011 (e.g., a heart valve-type valve) located distally to regulate unidirectional flow into or out of the system.

In some embodiments, instructions stored on one or more non-transitory computer-readable media are configured to synchronize the inflation and deflation cycles of descending aorta pump second configuration 5000 with the patient's cardiac cycle, as discussed in relation to FIG. 48, thereby preventing interference with intrinsic heart output. In some embodiments, during diastole, all inflatable elements remain in a deflated state, allowing unrestricted entry of intrinsic blood from the heart into the pumping chamber. In some embodiments, pump elements 5002 and 5003 are configured to inflate sequentially during systole to drive circulation in opposing directions—caudally and cranially—depending on the programmed sequence.

In some embodiments, the pump includes pump elements 5002 and 5003 configured with varying wall thicknesses or density to control inflation dynamics. In some embodiments, gas lumen and fill lumen 5041 are configured to deliver inflation media through fill orifices 5042 to the inflatable elements. In some embodiments, the system includes internal rib structures configured to introduce varying resistance across the balloon surface to achieve controlled inflation directionality. In some embodiments, the rib geometry or wall variation is engineered to preferentially expand one section of a pump element before another, resulting in directional flow control.

In some embodiments, descending aorta pump second configuration 5000 is configured to optimize blood perfusion to both the upper and lower extremities by leveraging a staged inflation sequence across pump elements 5002 and 5003. In some embodiments, the system uses multi-leaf polymer valve 5011 positioned at the distal end of the chamber to enforce directional flow during cranially directed pumping phases. In some embodiments, valve 5011 is collapsible and constructed from a biocompatible material compatible with delivery through a 10-12 French sheath.

In some embodiments, during a caudal pumping cycle, pump element 5002 inflates to displace blood distally through blood flow outlet 5010, while first check element 5001 prevents retrograde entry. In some embodiments, during a cranial pumping cycle, pump element 5003 inflates to displace blood proximally toward the aortic arch through blood flow outlet 5020, while multi-leaf polymer valve 5011 closes to enforce unidirectional flow. In some embodiments, each pump element is independently addressable through a gas regulation system configured to manage pressure and volume with precision.

In some embodiments, the control logic of descending aorta pump second configuration 5000 includes programmable instructions for selectively adjusting the inflation volume, speed, and sequencing of pump elements 5002 and 5003 to meet patient-specific hemodynamic needs. In some embodiments, operational cycle 5030 is defined based on systole and diastole timing and used to coordinate balloon actuation. In some embodiments, the system optionally includes sensors and/or feedback loops to dynamically adjust operation in real time. In some embodiments, by alternating or customizing directional flow patterns using pump elements 5002 and 5003, the system is configured to maintain or improve circulation in clinical scenarios where traditional unidirectional pumps may not meet perfusion demands.

In some embodiments, open end 5004 is positioned at the proximal end of descending aorta pump second configuration 5000 and does not require an independent check element. In some embodiments, when descending aorta pump second configuration 5000 is deployed in the descending aorta, the native aortic valve functions as a unidirectional barrier that closes during diastole, thereby preventing retrograde flow toward the left ventricle. In some embodiments, the aortic valve thus serves as a physiologic check element, blocking reverse flow when pump element 5003 is actuated to displace blood proximally through open end 5004. In some embodiments, this configuration advantageously reduces device complexity while maintaining directional flow control using intrinsic cardiovascular structures.

In some embodiments, pump elements 5002 and 5003 are configured such that a portion of each element adjacent to the center of stent 5004 inflates first during actuation. In some embodiments, this inflation pattern is configured to restrict or substantially occlude the central region of the stent, thereby creating a sealed barrier that inhibits cross-flow between the opposing flow paths. In some embodiments, this controlled inflation toward the center of stent 5004 ensures that when one of the pump elements inflates to drive blood in a given direction, the other pump element remains deflated, and flow is isolated to the desired path. In some embodiments, this localized expansion at the stent center enhances directional flow and facilitates alternating cranial and caudal perfusion by maintaining discrete flow chambers during each phase of the operational cycle. In some embodiments, for any multi-pumping element arrangement, the controller is configured to initiate a first pump element before a second pump element, where fluid is pumped in one direction before the other. In some embodiments, the system is configured to execute both pumps simultaneously, forcing fluid in both directions at the same time.

FIG. 51 illustrates descending aorta pump third configuration 5100 (e.g., a bi-directional bi-flow heart assist pump in the descending aorta) for improved systemic blood perfusion, in accordance with some embodiments. In some embodiments, descending aorta pump third configuration 5100 includes a stent-based housing containing two opposed directionally inflatable pump elements 5102 and 5103 (e.g., pumping balloons) separated by an inflatable chamber separator 5104 (e.g., separator balloon) that defines distinct pump chambers 1 and 2. In some embodiments, multi-leaf valve 5111 (e.g., a heart valve-type polymer valve) is integrated into the proximal end of the pump housing to regulate unidirectional flow during operational cycles.

In some embodiments, descending aorta pump third configuration 5100 is deployed such that open end 5120 faces proximally toward the aortic arch. In some embodiments, no additional check element is required at open end 5120 because the native aortic valve serves as a functional check valve, closing during diastole to prevent retrograde flow into the heart. In some embodiments, this use of the aortic valve as a physiologic check valve allows descending aorta pump third configuration 5100 to direct flow proximally through open end 5120 during a systolic pumping phase without requiring added mechanical components.

In some embodiments, the system is configured to execute an operational cycle 5130 aligned with the cardiac cycle, wherein all pump elements (e.g., 5102, 5103, 5104) are in a deflated state during diastole, allowing intrinsic cardiac output to fill the pump chambers. In some embodiments, during systole, the chamber separator 5104 is configured to inflate first to segment the internal space of the pump housing. In some embodiments, this inflation creates two isolated volumes within pump chamber 1 and pump chamber 2, allowing pump elements 5102 and 5103 to drive blood in opposing directions without pressure loss or mixing. In some embodiments, pump element 5102 inflates to drive caudal flow through blood flow outlet 5110, while pump element 5103 inflates to drive cranial flow through open end 5120.

In some embodiments, the inflation sequence of descending aorta pump third configuration 5100 begins with inflation of inflatable chamber separator 5105 (e.g., an inflatable central balloon) to divide the internal volume of the stent into two isolated pump chambers. In some embodiments, the inflation of chamber separator 5105 establishes a functional barrier that prevents fluid crossover between pump chamber 1 and pump chamber 2. Once the chamber is divided, first pump element 5102 and second pump element 5103 (e.g., directionally inflatable balloons) are inflated substantially simultaneously to drive blood flow in opposing directions. In some embodiments, first pump element 5102 is configured to displace blood distally through blood flow outlet 5110, while second pump element 5103 is configured to displace blood proximally through open end 5120. In some embodiments, valve 5111 (e.g., multi-leaf polymer valve) regulates inflow or prevents backflow during cranial-directed pumping. This coordinated inflation sequence enables bi-directional perfusion synchronized with the cardiac cycle while maintaining fluidic isolation between forward and reverse flow paths. While the term simultaneously is used herein with reference to bi-directional flow, in any of the examples of FIGS. 47-58, the timing between actuation of pumping elements, and/or time of bi-directional flow initiation, can be varied within a timeframe where the aortic valve is closed.

In some embodiments, fill lumens 5141 and fill orifices 5142 are configured to deliver inflation media to the respective pump elements and the chamber separator. In some embodiments, the fluid (e.g., gas) delivery system includes fill orifices of differing sizes or geometries to control the rate and sequence of inflation for each balloon. For example, chamber separator 5104 may include a larger orifice to ensure earlier inflation, establishing chamber isolation prior to directional flow generation by pump elements 5102 and 5103.

In some embodiments, the stent geometry of descending aorta pump third configuration 5100 is configured to vary along its length to accommodate differing balloon sizes or inflated volumes. In some embodiments, the stent may taper, bulge, or include segmented strut patterns to allow for simultaneous but asymmetrical inflation of pump elements 5102 and 5103. In some embodiments, this structural flexibility supports individualized tuning of cranial and caudal perfusion volumes, adapting to a patient's unique hemodynamic requirements. In some embodiments, the stent geometry is selected based on aortic anatomy to optimize chamber scaling, prevent stent migration, and reduce pressure losses during bi-directional operation.

In some embodiments, descending aorta pump third configuration 5100 supports a range of flow profiles including symmetric flow, alternating directional flow, or asymmetric persistent flow modes. In some embodiments, the programmable control logic is configured to vary inflation timing, duration, and volume to prioritize upper body or lower body circulation based on sensed physiologic demands or clinical settings.

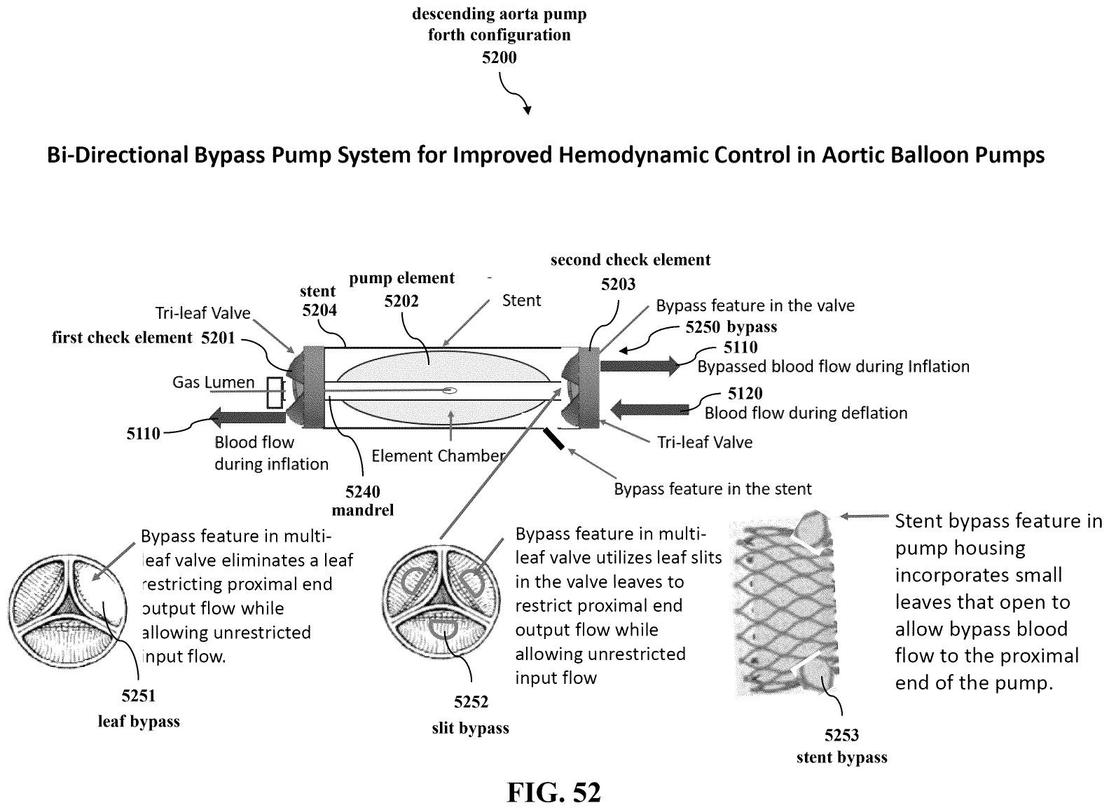

FIG. 52 illustrates descending aorta pump fourth configuration 5200 (e.g., a bi-directional bypass pump system for improved hemodynamic control in aortic balloon pumps), in accordance with some embodiments. In some embodiments, descending aorta pump fourth configuration 5200 includes pump element 5202 (e.g., an inflatable balloon) disposed within an element chamber supported by stent 5204. In some embodiments, pump element 5202 is positioned between first check element 5201 and second check element 5203 (e.g., collapsible valves). In some embodiments, the system includes gas lumen and inflation tubing for delivering pressurized gas or fluid to pump element 5202 via a central mandrel 5240, synchronized with the cardiac cycle.

In some embodiments, during diastole, all inflatable elements remain deflated, allowing unrestricted cardiac output to enter the element chamber. In some embodiments, during systole, pump element 5202 inflates and displaces blood both proximally and distally, with flow regulation achieved through the use of multiple bypass mechanisms. In some embodiments, blood is directed toward the lower extremities via second check element 5203 through blood flow outlet 5120. In some embodiments, a portion of the pressurized blood is redirected through bypass 5250 toward the proximal end via blood flow outlet 5110 to support perfusion of the upper body and brain.

In some embodiments, descending aorta pump fourth configuration 5200 includes one or more bypass mechanisms configured to enable fluid generated by pressure inside the stent housing to exit the housing and/or pass through one or more check valves, such as second check element 5203. In some embodiments, these bypass mechanisms are configured to allow a portion of blood displaced during pump element 5202 inflation to be redirected toward the proximal aorta and aortic arch, thereby improving upper body and cerebral perfusion during systolic phases.

In some embodiments, leaf bypass 5251 includes an orifice formed in one or more leaflets of a multi-leaf valve. In some embodiments, the size, shape, or placement of the orifice determines the amount of fluid allowed to flow through the check valve in the reverse direction toward the aortic arch. In some embodiments, the orifice is fixed in diameter and permits a calibrated degree of bypass flow without requiring leaflet displacement. In some embodiments, a leaf orifice is formed by a missing leaf in a multi-leaf check element.

In some embodiments, slit bypass 5252 includes one or more slits formed in the material of a multi-leaf valve. In some embodiments, the slits are configured to expand or open under internal pressure generated during inflation of pump element 5202, thereby allowing fluid to pass through the valve and into the proximal circulation. In some embodiments, the slit bypass provides a flow pathway that is normally closed at rest and opens elastically under pressure, with the degree of expansion determining the resulting bypass volume.

In some embodiments, stent bypass 5253 includes one or more flaps or openings in the stent structure configured to open under internal pressure, allowing blood to exit the housing through the stent wall. In some embodiments, stent bypass 5253 is configured with resilient elements that close passively when the internal pressure decreases, thereby preventing retrograde flow into the pump during diastole. In some embodiments, stent bypass 5253 may be formed in a tapered area and/or an area of the stent separated from the aortic wall when deployed. In some embodiments, stent bypass 5253 includes one or more orifices in the stint 5304.

In some embodiments, the bypass 5250 (e.g., 5251, 5252, and 5253) are configured individually or in combination to enable controlled redirection of flow during systole, with flow path selection determined by pressure dynamics, leaflet compliance, slit geometry, or orifice size. In some embodiments, the combined bypass configuration supports both forward (caudal) and backward (cranial) flow profiles to enhance balanced systemic perfusion and reduce the risk of under-perfusion to upper-body regions during active pump operation.

In some embodiments, descending aorta pump fourth configuration 5200 is configured to overcome limitations associated with unidirectional balloon pumps deployed in the descending aorta by enabling balanced perfusion to both upper and lower extremities. In some embodiments, this system reduces preferential lower-body perfusion by introducing resistance-moderated bypass flow pathways that direct blood volume back toward the proximal end during each systolic pump cycle. In some embodiments, the control mechanism is configured to time inflation with the patient's intrinsic cardiac rhythm, and, in some embodiments, valve geometry at the proximal end is selectively tuned to provide desired bypass flow.

In some embodiments, the system includes programmable flow control through any combination of the valve bypass features or stent-integrated pathways. In some embodiments, the bypass system is configured to enhance cerebral perfusion while still allowing sufficient output to the lower body through second check element 5203. In some embodiments, by combining pump element 5202, mandrel 5240, and multiple bypass features, descending aorta pump fourth configuration 5200 offers improved hemodynamic performance across varying physiologic conditions.

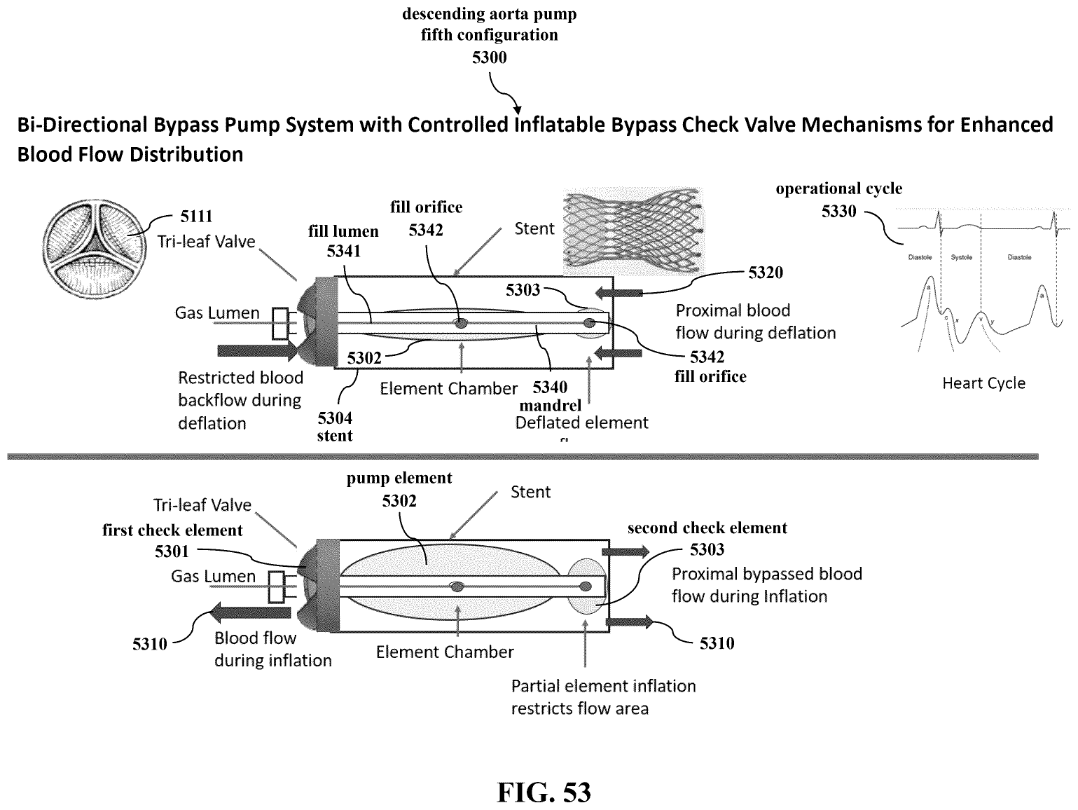

FIG. 53 illustrates descending aorta pump fifth configuration 5300 (e.g., a bi-directional bypass pump system with controlled inflatable check valve mechanisms for enhanced blood flow distribution), in accordance with some embodiments. In some embodiments, descending aorta pump fifth configuration 5300 includes pump element 5302 (e.g., an inflatable balloon) disposed within a stent-supported element chamber between first check element 5301 and second check element 5303. In some embodiments, first check element 5301 includes a tri-leaflet polymer valve, and second check element 5303 is an inflatable check valve configured to act as a controllable bypass mechanism at the proximal end of the system.

In some embodiments, during the diastolic phase, all inflatable elements are in a deflated state, permitting unrestricted entry of native heart blood into the element chamber. In some embodiments, pump element 5302 inflates during systole to displace blood in both directions, with flow regulated by the collapsible valve at the distal end and the inflatable check valve at the proximal end. In some embodiments, second check element 5303 is configured to operate as an adjustable bypass valve, wherein the valve can be uninflated, partially inflated, or fully inflated to control the amount of blood allowed to flow through proximal outlet 5320 toward the aortic arch.

In some embodiments, when second check element 5303 remains deflated, blood is permitted to exit the proximal end of the stent housing substantially unimpeded during the systolic phase, facilitating increased cerebral perfusion. In some embodiments, when second check element 5303 is partially inflated, it restricts but does not completely block flow through proximal outlet 5320, allowing a predetermined volume of blood to bypass through the proximal end while maintaining flow through the distal outlet. In some embodiments, partial inflation is achieved through control of gas pressure delivered via fill lumen 5341 and fill orifice 5342. In some embodiments, full inflation of second check element 5303 substantially seals the proximal end, redirecting the entirety of the output flow toward the lower extremities via the distal check valve. In some embodiments, at fully inflated the second check element 5303 does not seal the end of the pump chamber, where a predetermined amount of fluid can bypass the fully inflated second check element 5301.

In some embodiments, the system includes mandrel 5340 configured to provide structural support to pump element 5302 and second check element 5303. In some embodiments, the inflation and deflation of each element are coordinated using an operational cycle 5330 synchronized to the patient's cardiac rhythm. In some embodiments, variable fill orifice sizing between pump element 5302 and second check element 5301 facilitates sequential or staggered inflation, enabling tailored flow distribution based on desired perfusion targets.

In some embodiments, descending aorta pump fifth configuration 5300 allows for dynamic tuning of blood flow distribution between cranial and caudal regions by modifying the inflation state of second check element 5301. This provides clinicians with an adaptive tool for optimizing perfusion, particularly for cerebral protection. In some embodiments, the inflatable proximal valve is configured to fully deflate during the return to diastole, eliminating barriers to native blood entry and ensuring chamber refill is not impeded.

In some embodiments, the system's ability to modulate second check element 5301 through partial or full inflation presents a significant advantage over fixed bypass or passive check valves, as it provides programmable, hemodynamically responsive flow control. This functionality is useful in scenarios where perfusion balance is delicate, such as during cerebral support, and allows descending aorta pump fifth configuration 5300 to support a broader range of physiologic and clinical needs.

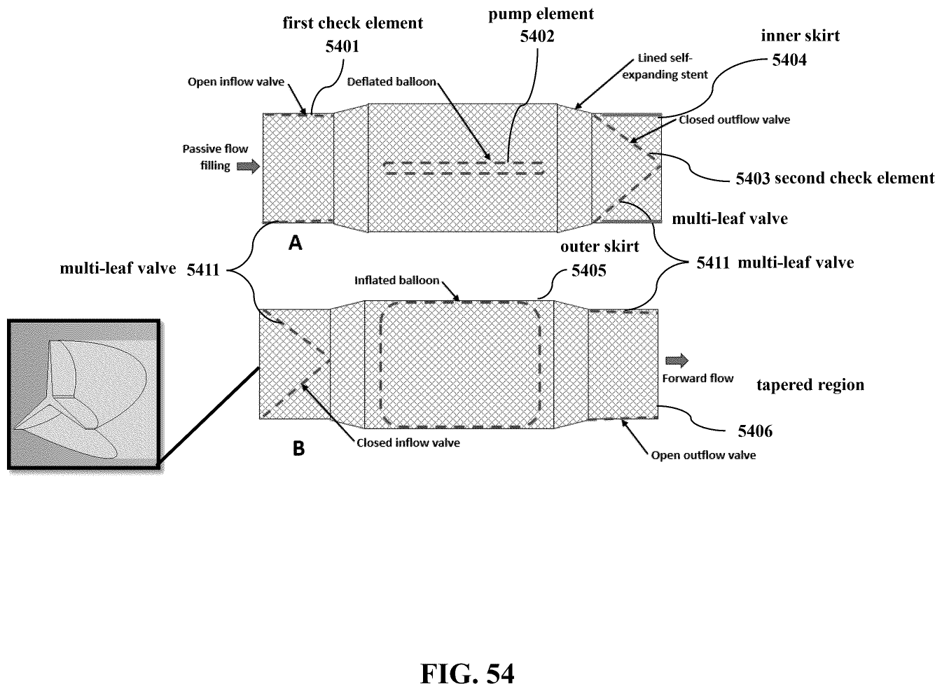

FIG. 54 illustrates descending aorta pump configuration similar to 5200 (e.g., a heart assist pump system with tri-leaflet valve integration and a single balloon element), in accordance with some embodiments. In some embodiments, descending aorta pump sixth configuration 5400 includes pump element 5402 (e.g., a single inflatable balloon) disposed between first check element 5401 and second check element 5403 within a lined, self-expanding stent housing. In some embodiments, first check element 5401 and second check element 5403 are multi-leaf valve assemblies 5411 (e.g., tri-leaflet polymer valves) integrated into tapered regions of the stent to maintain functionality even when full stent expansion is not achieved.

In some embodiments, stage A depicts the diastolic phase of the operational cycle in which pump element 5402 is in a deflated state, allowing passive inflow of intrinsic blood through first check element 5401. In some embodiments, during this phase, first check element 5401 is open while second check element 5403 is closed, maintaining unidirectional flow and enabling blood to fill the interior chamber of the stent housing.

In some embodiments, stage B illustrates the systolic phase in which pump element 5402 is inflated using externally delivered gas, such as helium. In some embodiments, the inflation of pump element 5402 increases internal chamber pressure and causes second check element 5403 to open while first check element 5401 closes. This configuration directs forward flow toward the lower extremities through second check element 5403, ensuring consistent perfusion in synchronization with the cardiac cycle.

In some embodiments, multi-leaf valves 5411 are fabricated from thin biocompatible polymer material, with each leaflet configured to collapse against the mandrel or stent interior when closed. In some embodiments, the valves include pre-folded geometries or engineered crease lines to facilitate compact folding during percutaneous delivery through a 10-12 French sheath and controlled unfolding upon deployment. In some embodiments, the valves are integrated into regions of reduced stent diameter relative to the main stent body, providing a functional seal even in anatomically restricted or non-uniform aortic segments.

In some embodiments, pump element 5402 is configured to cycle between inflated and deflated states in response to control signals generated by an external controller. In some embodiments, this inflation-deflation cycle operates in counter-pulsation with the heart or is programmable to alternate with the cardiac rhythm, as previously described

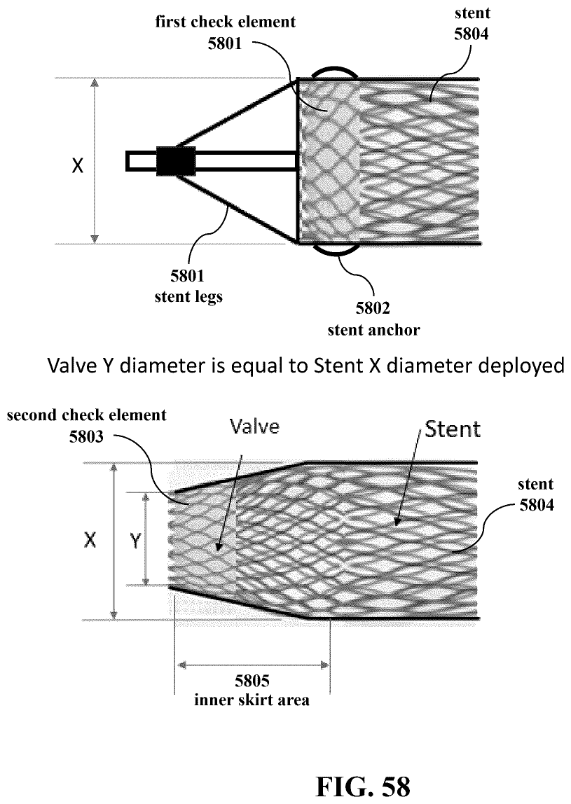

In some embodiments, an inner circumferential skirt is positioned along the inner surface of the stent, adjacent to and surrounding first check element 5401 and/or second check element 5403. This skirt is configured to interface directly with the multi-leaf valve 5411, providing a compliant sealing surface that supports valve coaptation during closure and reinforces leaflet seating. Further details of non-limiting inner skirt deployment configurations are discussed in relation to FIG. 58.

In some embodiments, the inner skirt 5404 is constructed from a biocompatible, elastomeric material, such as a polymer membrane or coated mesh, that lines the inner perimeter of the stent at the valve junction. When the leaflets of the tri-leaflet valve collapse inward (as seen during systole at the inflow valve and during diastole at the outflow valve), they contact the inner skirt to form a completely or substantially completely fluid-tight seal, helping prevent or greatly reduce perivalvular backflow through the stent wall. In some embodiments, the inner skirt may also absorb minor deformation in the stent structure caused by aortic wall irregularities, maintaining valve performance in anatomically constrained or calcified regions.

In some embodiments, an outer skirt 5405 is positioned on the outer surface of the stent, typically between or adjacent to the zones occupied by multi-leaf valve 5411 and pump element 5402. This outer skirt is configured to engage with the inner wall of the aorta and serves primarily as an anti-migration and stabilization feature.

In some embodiments, the outer skirt is formed from a flexible polymer membrane or woven fabric attached to the stent frame. This skirt is configured to deploy outward upon stent expansion and to conform to the aortic lumen, improving radial engagement. In some embodiments, the outer skirt may include textured surface features, such as ridges, micro-spikes, raised projections, or embedded filament loops, configured to increase friction and resist axial movement or rotational displacement of the pump during repetitive balloon actuation.

In some embodiments, the outer skirt performs a protective function, minimizing abrasion or trauma to the aortic wall by distributing mechanical loads caused by inflation and deflation of pump element 5402. In some embodiments, the skirt membrane is coated with a low-friction biocompatible surface, such as PTFE or silicone-based compounds, to further reduce wall irritation during prolonged use. In some embodiments, this outer skirt contributes to secure anchoring of the pump in cases where the aortic anatomy is non-uniform or subject to pressure variations, and helps prevent embolization or displacement during transient loss of balloon pressure or during catheter manipulation.

In some embodiments, a skirt's material properties, such as thickness, surface compliance, or hydrophilicity, may be tailored to enhance leaflet sealing under dynamic loading conditions, ensuring reliable valve function even when the stent is not fully expanded. In some embodiments, the inner skirt enables unidirectional control of blood flow through the pump while reducing mechanical stress on the valve leaflets.

Still referring to FIG. 54, in some embodiments, the system is a temporary percutaneous implanted system intended to assist the diseased native left ventricle by providing enhanced flow and cardiac output. In some embodiments, the system is configured to be implanted for a sub-chronic period of up to 42 days, or longer if configured with materials configured for extended durations. In some embodiments, implanted components include at least one inflatable pumping element (balloon) and one deflatable pumping element, where each is driven by an external pumping drive platform. In some embodiments, the pumping element is housed within a self-expanding stent pump chamber that is configured to be delivered and deployed through a minimally invasive catheter system within the vascular system.

In some embodiments, the system includes a catheter. Some embodiments include a step of inserting the system (e.g., a catheter with or without a stent) into the aorta. Some embodiments include a step of creating an opening in the leg and/or arm to insert the system. Some embodiments include a step of guiding the pump (e.g., plurality of pump elements) to the aorta.

In some embodiments, the system is configured to be a minimally invasive and delivered through a small incision. In some embodiments, the system is configured to be delivered to the descending aorta using a femoral artery access, typically used for Transcatheter Aortic Valve Replacement (TAVR). In some embodiments, the system includes an element contained within a lined self-expanding stent pump chamber that is configured to be compressed to a small diameter to fit within a 10 to 12 French catheter. In some embodiments, according to a method of use, once the catheter is advanced to the target deployment location, the stent pump chamber and element are configured to be released from the catheter, allowing the stent pump chamber to expand. In some embodiments, the catheter sheath is configured to then be retracted to leave the existing blood pump system in place.

In some embodiments, the check valves in the stint pump chamber are configured to keep flow moving away from the heart and prevent backflow. Preventing backflow as used herein includes complete prevention as well as preventing virtually all backflow, recognizing that a perfect seal is often not accomplished in clinical use. In some embodiments, during diastole, when one or more pumping elements are in the deflated configuration, the stint pump chamber is configured to enable passive filling of the system from blood in the ascending aorta, the inflow valve is open, and the outflow valve is configured to be closed due to systemic pressure in the arterial system downstream of the system. In some embodiments, at the start of systole, the element is inflated, causing the inflow valve to close and the outflow valve to open, ejecting the blood volume displaced from the inflated element as forward flow into the arterial vasculature. In some embodiments, although actuating the element pump in sync with the heart cycle is a normal mode, it is also possible to use counter-pulsation. In some embodiments, when the heart relaxes (diastole), the balloon inflates, pushing blood back toward the heart to improve coronary perfusion (feeding the heart muscle itself). In some embodiments, when the heart contracts (systole), the balloon deflates rapidly, creating a vacuum effect that reduces afterload (makes it easier for the heart to pump blood out).

In some embodiments, the element inflation and deflation are configured to be synchronized with the heartbeat by the control platform (FIG. 1) connected to the pumping element by a small diameter fill lumen. In some embodiments, the element supply tube is configured to be routed to the element through the distal outflow end of the pump housing so that when the outflow valve leaflets close and couple with each other, they also close around the tube to seal against backflow. In some embodiments, the output from the pump system is configured to be controlled by the element inflation volume and the beat rate through an EKG and pressure sensor input module in controller 102.

In some embodiments, the system includes a simpler single stented single element with other mechanisms replaced by simple inflow and outflow heart-type check valves. In some embodiments, the inflow and outflow valves are configured as a tri-leaflet prosthetic aortic valve. For example, in some embodiments, flexible tri-leaflet valves constructed from a thin polymer is mounted within the PHAP stent pump housing frame. In some embodiments, the system includes inflow and outflow valves configured and attached to the element pump chamber stent.

TAVR valves are designed for crimping and delivery in 15 to 21 French catheters. In some embodiments, the polymer used for the the present system allows for leaflet thicknesses on the order of 60-125 micrometers, with 100 micrometers enabling the system to be inserted into a 10-12 French catheter, marking a significant improvement over the prior art, as smaller catheters are much easier to manipulate.

An aspect that enables such low-profile delivery, in accordance with some embodiments, is the use of a thin-film multi-leaf valve 5411 (e.g., tri-leaflet check valve) integrated into one or both ends of the stent housing. In some embodiments, the leaflets of multi-leaf valve 5411 are fabricated from a polymeric material with a thickness of less than 125 microns, significantly thinner than conventional percutaneous heart valve systems, which often exceed 200 microns. This reduced leaflet thickness allows the valve, in its folded or pre-delivery configuration, to collapse into a much smaller diameter, thereby enabling the full assembly to fit within a compact catheter sheath for percutaneous navigation.

In some embodiments, the use of a single pumping element (e.g., pump element 5402) contained within a lined self-expanding stent also contributes to the reduced delivery profile. Because the system includes only one inflatable chamber and two check elements (rather than a long series of balloons or impeller blades), the overall axial length and mechanical complexity of the device are minimized. This allows the stent chamber and pump structure to be crimped and compressed as a unit prior to deployment.

In some embodiments, once the catheter reaches the target deployment site within the descending aorta, the stent is configured to self-expand upon release from the sheath. In some embodiments, the stent is configured to expand up to 25 mm, as a non-limiting example, in diameter to conform to a typical aortic lumen. However, in anatomies where the aorta is narrower, such as 20 mm, the stent may not fully expand. In such cases, the configuration of multi-leaf valve 5411 is configured to preserve functionality even under partial expansion conditions. In some embodiments, this is achieved by locating multi-leaf valve 5411 in a tapered region of the stent, such that the valve sits in a zone that maintains a smaller diameter (e.g., 20 mm) regardless of full stent expansion. Further details of a non-limiting example of a tapered area for a stent are discussed in reference to FIG. 58.

In some embodiments, the leaflets of multi-leaf valve 5411 maintain their ability to open and close around a central mandrel or guidewire due to their reduced thickness and high flexibility. Although leaflet coaptation efficiency may be slightly reduced under lower expansion, the valve remains sufficiently competent to regulate directional blood flow and prevent significant regurgitation during pump operation. This configuration flexibility ensures consistent valve performance across a range of aortic diameters, expanding the applicability of the system to diverse patient anatomies without requiring larger sheath sizes.

In some embodiments, the valve skirt (e.g., inner skirt between the valve and stent frame) provides sealing support for the partially deployed valve, further reinforcing leaflet alignment and reducing perivalvular leakage in constrained environments.

Collectively, the combination of a single inflatable pump element, a thin-leaf multi-leaf valve with pre-folded geometry, and a tapered valve region integrated into a self-expanding stent allows the entire heart assist system to be delivered through a 10-12 French catheter. This represents a significant advancement over impeller-based devices, which often require larger sheaths (e.g., 15-21 French), and enhances procedural safety by minimizing access site complications, vascular trauma, and time to deployment.

In some embodiments, the polymer selected for the valve leaflets is blood compatible and/or non-thrombogenic, with mechanical integrity for adequate durability. In some embodiments, the polymers for heart valves are configured to survive the equivalent of a minimum of 5 years of durability. In some embodiments, the system includes a service life of 43+ days, which is less than 5% of a typical heart valve's lifespan. Therefore, the configuration and material requirements for durability are not as demanding as those for long term use, and a number of available medical polymers are available as a benefit from limiting the lifespan.

In some embodiments, incorporating the inflow and outflow valves results in advantages such as hemodynamic efficiency, durability, case of crimping, blood compatibility, and low cost. In some embodiments, combinations of the system that include polymer check valves allow for the creation of a valve configured to seal on a mandrel, include a shape of the expanding pump chamber to enhance directional flow, allow for different size valves for inflow and outflow, include multiple gas outlets for balanced inflation, and include vacuum assist for greater control.

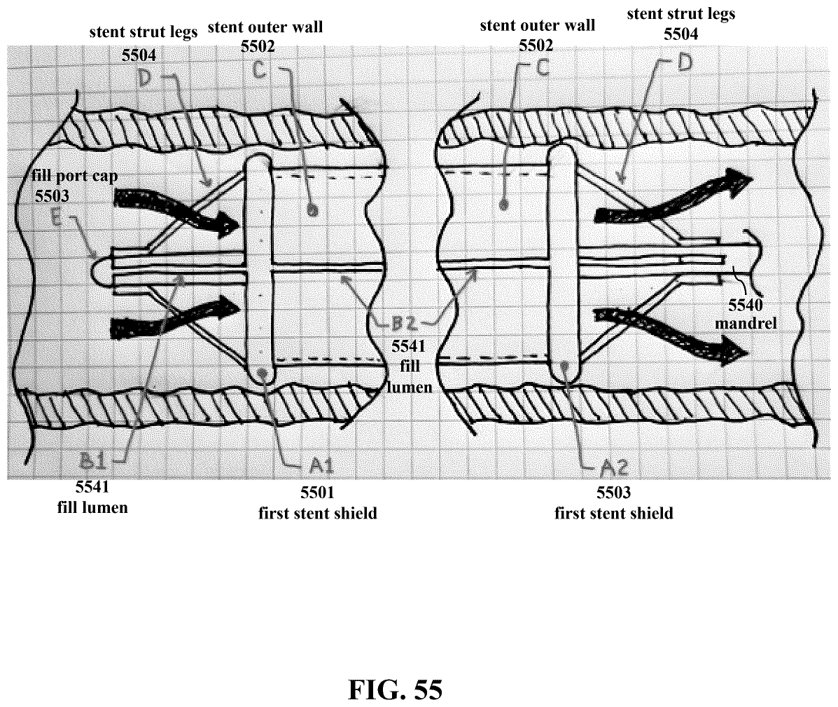

FIG. 55 illustrates a descending aorta pump shield configuration that can be applied to any system configuration described herein, and/or combinations thereof, in accordance with some embodiments. FIG. 55 illustrates a descending aorta pump seventh configuration 5500, including first stent shield 5501 and second stent shield 5503 disposed along an outer surface of stent outer wall 5502, in accordance with some embodiments. In some embodiments, first stent shield 5501 and second stent shield 5503 are inflatable structures (e.g., stent-mounted balloon rings) configured to surround stent outer wall 5502 and provide a soft, compliant interface between the stent and the aortic wall.

In some embodiments, the inflatable stent shields are positioned on the proximal and distal ends of the pump system and are configured to protect the aorta from direct contact with the stent which is typically formed of rigid metallic material such as nitinol, but may include skirt material as previously described. In some embodiments, the inflatable stent shields define a low-profile external cushion that reduces pressure concentration against the vascular wall during pump operation, particularly in high-cycle systems.

In some embodiments, first stent shield 5501 and second stent shield 5503 are selectively inflated via internal conduits routed through a central catheter shaft. In some embodiments, fill lumen 5541 is configured to carry inflation media to first stent shield 5501, and fill lumen 5541 is configured to carry inflation media to second stent shield 5503. In some embodiments, fill port cap 5503 includes valve-controlled access to fill lumen 5541 and/or fill lumen 5541 and may be positioned at the catheter tip or hub to interface with a controller or manual injection system.

In some embodiments, the stent shields are filled with a gas or liquid medium, such as helium, CO₂, or saline, which is delivered via mandrel 5540 or an internal fluid lumen structure, as described above, enabling real-time pressurization of each balloon after deployment. In some embodiments, mandrel 5540 includes integrated fill channels or radial outlets to direct flow into the volume between the shield material and the stent wall.

In some embodiments, the inflatable stent shields perform a sealing function, creating a boundary at both ends of the pump chamber. This function enables the stent pump to operate effectively even when the main stent outer wall 5502 is not fully sealed or coated. In some embodiments, first stent shield 5501 is positioned to limit or block retrograde flow at the proximal region of the pump chamber, enhancing pump operation. In some embodiments, the shields may also serve to retain the pump device within a defined aortic region by creating localized fixation pressure between the expanded shield and the aortic lumen wall.

In some embodiments, the compliant nature of first stent shield 5501 and second stent shield 5503 reduces the mechanical stress imposed on the aortic wall, especially during long-duration operation or repeated pulsatile expansion of internal pump components. In some embodiments, the inflatable stent shields reduce the area of contact between the rigid stent frame and the vessel wall, enabling extended indwelling time and minimizing the risk of dissection, irritation, and/or erosion.

In some embodiments, the materials used to form the stent shields include biocompatible elastomers or thermoplastic polyurethane films with high cycle durability and thromboresistance. In some embodiments, the outer surface of the shields is textured or treated with surface coatings to further reduce migration or slippage during use. In some embodiments, first stent shield 5501 and second stent shield 5503 provide anatomical conformity, vascular protection, and hemodynamic stability within descending aorta pump seventh configuration 5500, enabling advanced control of pump sealing, anchoring, and flow modulation through minimally invasive deployment. In some embodiments, this inflatable stent shield configuration is applicable across a range of device types, including single-chamber pumps, bi-directional pumps, and systems employing external perfusion loops or sensors.

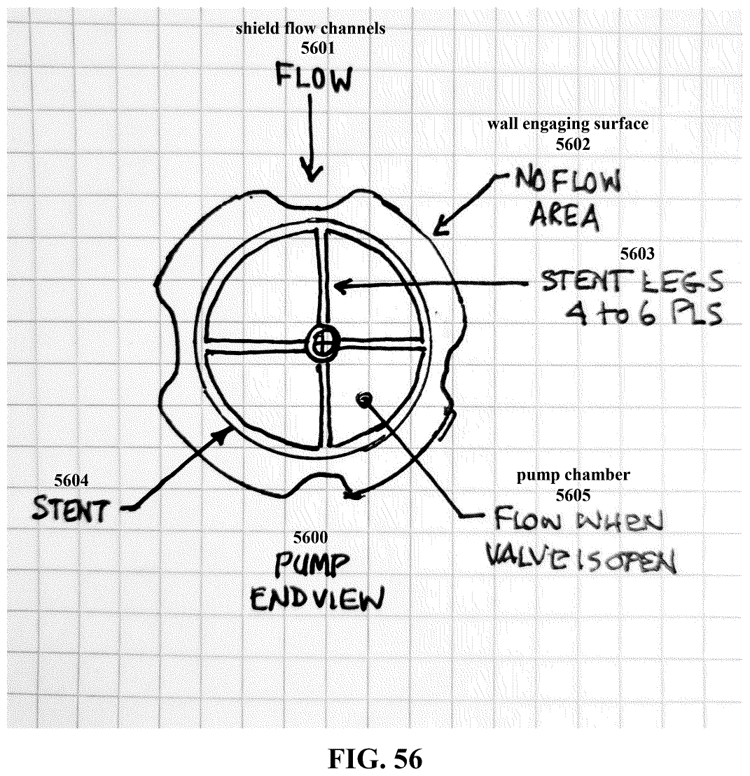

FIG. 56 illustrates an end view of FIG. 55, showing structural and functional details of the outer stent interface and bypass flow features, in accordance with some embodiments. In some embodiments, stent 5604 forms an expandable cylindrical framework for securing the pump system within the aorta, and includes a plurality of stent legs 5603 (e.g., four to six struts) radiating inward to support pump chamber 5605.

In some embodiments, the outer surface of stent 5604 defines a wall engaging surface 5602, configured to contact the inner surface of the aortic wall. In some embodiments, one or more shield flow channels 5601 are defined in or around wall engaging surface 5602, and are configured to allow trapped fluid, such as blood or perfusate, located between the stent and the vessel wall to escape or circulate. In some embodiments, this mitigates pooling or stagnation of blood within the no flow area 5602, which may otherwise promote thrombus formation during extended pump operation.

In some embodiments, pump chamber 5605 is aligned centrally and, when the pump element is deflated, permits axial blood flow 5610 in the forward direction during systole. In some embodiments, when the valve is closed, the configuration of the surrounding shield flow channels 5601 enables redirected flow to escape around the periphery of the device.

In some embodiments, the shield flow channels 5601 are integrally formed within the inflatable stent shield (e.g., as shown in FIG. 55) thereby defining discrete bypass segments that preserve hemodynamic circulation even in areas not directly contributing to pump flow. In some embodiments, the shield flow channels 5601 are configured to enable perfusion of adjacent vascular structures, such as intercostal arteries or spinal cord branches, and contribute to the overall physiological compatibility of the pump, providing a balance between fixation, sealing, and perfusion continuity.

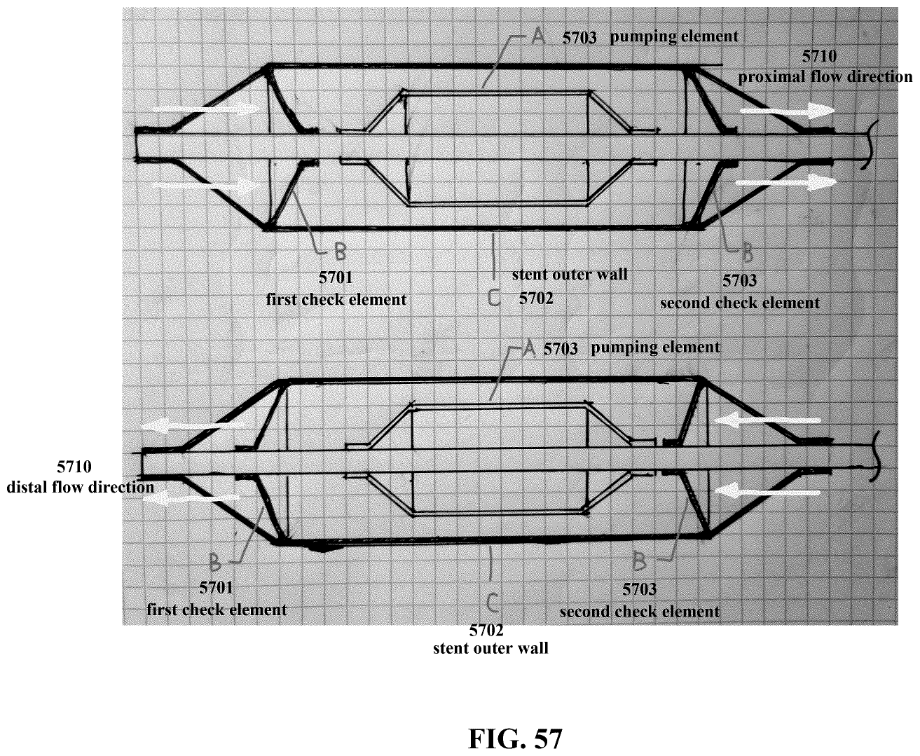

FIG. 57 illustrates a directional flow control configuration of a descending aorta pump, demonstrating how the orientation of check elements within the system determines the direction of blood flow through the pump chamber, in accordance with some embodiments. In some embodiments, stent outer wall 5702 supports a centrally located pumping element 5703, bounded at either end by one or more check valve elements.

The top portion of FIG. 57 shows a configuration in which blood is directed in the proximal flow direction 5710, with first check element 5701 located at the distal end and second check element 5703 positioned at the proximal end, relative to the direction of insertion. In this configuration, second check element 5703 is oriented to allow fluid to exit proximally during systolic pumping, while first check element 5701 is oriented to prevent retrograde flow back into the distal end during the pumping phase.

Conversely, the lower portion of FIG. 57 demonstrates a mirrored configuration in which blood flows in the distal flow direction 5710. Here, the orientation of first check element 5701 and second check element 5703 are reversed relative to the pump chamber. In this arrangement, first check element 5701 permits forward flow, which may be a useful configuration for a different part of the body. In some embodiments, the ability to reorient the check elements within the stent allows a clinician or device manufacturer to select a target flow direction based on clinical need. In some embodiments, the system may be preassembled in one configuration or include interchangeable valve components that permit reconfiguration before or during deployment.

In both configurations, pumping element 5703 is centrally located and configured to undergo inflation and deflation cycles that drive blood through the directionally restricted channel defined by the check elements. In some embodiments, the pump chamber is bounded by stent outer wall 5702, which maintains the shape and position of internal components relative to the surrounding vascular anatomy and may include any of the features previously described.

FIG. 58 illustrates non-limiting configurations of check valve arrangements that may be used in conjunction with any of the pump systems and stent assemblies described in previous Figures, in accordance with some embodiments. In some embodiments, the first check element 5801 is positioned at a proximal end of the stent 5804, which includes stent legs 5801 and a stent anchor 5802. In some embodiments, the stent legs 5801 extend outward from a central housing structure to engage the vessel wall or catheter interface during operation. In some embodiments, the stent anchor 5802 is configured to couple the stent to the aortic wall to maintain axial alignment and radial stability. In some embodiments, the stent anchor 5802 includes a stent shield such as described in relation to FIG. 55.

In some embodiments, the first check element 5801 includes a diameter equal to a deployed diameter X of the stent 5804. In some embodiments, the valve leaflets coupled to the first check element 5801 include a leaflet thickness ranging from approximately 80 microns to approximately 125 microns, with a thickness of approximately 100 microns found to be suitable for most applications. In some embodiments, a second check element 5803 is positioned within a valve region defined at an end of the stent 5804. In some embodiments, the second check element 5803 includes a diameter Y, where the check element diameter Y ranges from 70% to 99% of the deployed stent diameter X, with a diameter of approximately 85% of the stent diameter found to be suitable for most applications.

In some embodiments, the valve region (e.g., pump chamber first end or second end) includes an inner skirt area 5805 positioned between the second check element 5803 and the stent 5804. In some embodiments, the inner skirt area 5805 is configured to manage blood flow transition through the valve by providing a tapered and/or compliant (elastically deformable under applied force) conduit that minimizes turbulence and improves sealing. In some embodiments, the check element and skirt arrangement are configured to collapse during insertion and expand during deployment to conform with the stent 5804 and surrounding vascular anatomy. In some embodiments, the valve leaflets associated with the second check element 5803 also include a leaflet thickness ranging from approximately 80 microns to approximately 125 microns, with a thickness of approximately 100 microns found to be suitable insertion in a 10 to 12 French catheter.

In some embodiments, the stent 5804 includes a mesh structure that is configured to support valve operation by providing a rigid or semi-rigid scaffold for the check elements. In some embodiments, either the first check element 5801 or the second check element 5803 may be used with stent 5804 to implement unidirectional or bidirectional flow control. In some embodiments, the dimensional relationships between valve diameter Y and stent diameter X are selected to ensure effective sealing, backflow prevention, and hemodynamic compatibility during pulsatile flow.

In some embodiments, a method of augmenting blood flow and improving systemic and cerebral perfusion includes providing a descending aorta pump, such as any described above. In some embodiments, the method includes providing a catheter, inflatable pump elements, check valves, a stent housing, and/or any structure described herein configured for deployment within the descending aorta. Some embodiments include a method step of preparing the system by assembling the catheter, stent housing, pump elements, check valves, and associated components. Some embodiments include a method step of preloading the pump elements and check valves into the stent housing, and/or compressing the stent housing to fit within a catheter sheath. Some embodiments include a method step of configuring the catheter with fill lumens and ports for delivering inflation media to the pump elements. Some embodiments include a method step of sterilizing and packaging the system for use in a clinical environment. Some embodiments include a method step of programming a controller with operational parameters including inflation and deflation sequences, cycle timing, and/or pressure thresholds based on patient-specific hemodynamic data, according to some embodiment described herein.

Some embodiments include a method step of creating a vascular access point, such as in the femoral artery, and inserting the catheter through the vasculature under imaging guidance. Some embodiments include a method step of advancing the catheter to the descending aorta and positioning the system relative to a target deployment site. Some embodiments include a method step of guiding the catheter using a guidewire to facilitate precise placement. Some embodiments include a method step of releasing the stent housing and pump elements from the catheter sheath, and/or allowing the stent housing to expand and conform to the inner wall of the aorta.

Some embodiments include a method step of deploying the stent housing by allowing it to expand radially to anchor the pump system within the descending aorta. Some embodiments include a method step of maintaining the pump elements and check valves in a deflated state during deployment to minimize resistance to blood flow. Some embodiments include a method step of providing the stent housing with expandable stent shields configured to create a compliant interface with the aortic wall, thereby reducing mechanical stress and promoting vascular protection. Some embodiments include a method step of positioning the system to align with the aortic anatomy, such that the pump elements are oriented to direct blood flow proximally toward the aortic arch or distally toward the lower extremities, based on programmed control logic.

Some embodiments include a method step of actuating the pump system by operating the controller to synchronize inflation and deflation of the pump elements with the cardiac cycle. Some embodiments include a method step of deflating the pump elements during diastole to allow passive blood inflow and inflating the pump elements during systole to displace blood in a desired direction. Some embodiments include a method step of generating proximal flow toward the aortic arch to enhance perfusion to the upper extremities and cerebral vasculature. Some embodiments include a method step of providing alternating directional flow to maintain balanced perfusion to both upper and lower extremities.

Some embodiments include a method step of monitoring real-time sensor data, such as pressure and flow rate, and dynamically adjusting pump operation in response to detected conditions. Some embodiments include a method step of diverting a portion of the blood flow toward the aortic arch using one or more bypass conduits to further improve cerebral perfusion. Some embodiments include a method step of utilizing pump elements having pleated surfaces or tapered shapes to enhance inflation dynamics and fluid displacement characteristics.

Some embodiments include a method step of removing the system by advancing the catheter sheath over the stent housing to compress the pump elements and stent for retraction. Some embodiments include a method step of withdrawing the system through the same vascular access point and disposing of the removed components according to medical waste protocols.

Some embodiments include a method step of improving systemic and cerebral perfusion by enabling bidirectional or alternating directional pumping that operates in synchronization with the cardiac cycle. Some embodiments include a method step of reducing procedural risk and recovery time by utilizing a catheter-delivered system adaptable to varied anatomical and physiological conditions.

With the above embodiments in mind, it is understood that the system is configured to implement various computer-implemented program steps involving data stored one or more non-transitory computer media according to some embodiments. In some embodiments, the above-described databases and models described throughout this disclosure are configured to store analytical models and other data on non-transitory computer-readable storage media within the computer system 2310 and on computer-readable storage media coupled to the computer system 2310 according to some embodiments. In addition, in some embodiments, the above-described applications of the system are stored on computer-readable storage media within the computer system 2310 and on computer-readable storage media coupled to the computer system 2310. In some embodiments, these operations are those requiring physical manipulation of structures including electrons, electrical charges, transistors, amplifiers, receivers, transmitters, and/or any conventional computer hardware in order to transform an electrical input into a different output. In some embodiments, these structures include one or more of electrical, electromagnetic, magnetic, optical, and/or magneto-optical signals capable of being stored, transferred, combined, compared, and otherwise manipulated. In some embodiments, the computer system 2310 comprises at least one computer readable medium 2336 coupled to at least one of at least one data source 2337a, at least one data storage 2337b, and/or at least one input/output 2337c. In some embodiments, the computer system 2310 is embodied as computer readable code on a computer readable medium 2336. In some embodiments, the computer readable medium 2336 includes any data storage that stores data, which is configured to thereafter be read by a computer (such as computer 2340). In some embodiments, the non-transitory computer readable medium 2336 includes any physical or material medium that is used to tangibly store the desired information, steps, and/or instructions and which is configured to be accessed by a computer 2340 or processor 2332. In some embodiments, the non-transitory computer readable medium 2336 includes hard drives, network attached storage (NAS), read-only memory, random-access memory, FLASH-based memory, CD-ROMs, CD-Rs, CD-RWs, DVDs, magnetic tapes, and/or other optical and non-optical data storage. In some embodiments, various other forms of computer-readable media 2336 are configured to transmit or carry instructions to one or more remote computers 2340 and/or at least one user 2331, including a router, private or public network, or other transmission or channel, both wired and wireless. In some embodiments, the software application modules 2338 are configured to send and receive data from a database (e.g., from a computer readable medium 2336 including data sources 2337a and data storage 2337b that comprises a database), and data is configured to be received by the software application modules 2338 from at least one other source. In some embodiments, at least one of the software application modules 2338 are configured to be implemented by the computer system 2310 to output data to at least one user 2331 via at least one graphical user interface rendered on at least one digital display.

In some embodiments, the one or more non-transitory computer readable 2336 media are distributed over a conventional computer network via the network interface 2335a where some embodiments stored the non-transitory computer readable medium are stored and executed in a distributed fashion. For example, in some embodiments, one or more components of the computer system 2310 are configured to send and/or receive data through a local area network (“LAN”) 2339a and/or an internet coupled network 2339b (e.g., such as a wireless internet). In some embodiments, the networks 2339a, 2339b include one or more wide area networks (“WAN”), direct connections (e.g., through a universal serial bus port), or other forms of computer-readable media 2336, and/or any combination thereof.

In some embodiments, components of the networks 2339a, 2339b include any number of personal computers 2340 which include for example desktop computers, laptop computers, and/or any fixed, generally non-mobile internet appliances coupled through the LAN 2339a. For example, some embodiments include one or more personal computers 2340, databases 2341, and/or servers 2342 coupled through the LAN 2339a that are configured for use by any type of user including an administrator. Some embodiments include one or more personal computers 2340 coupled through network 2339b. In some embodiments, one or more components of the computer system 2310 are configured to send or receive data through an internet network (e.g., such as network 2339b). For example, some embodiments include at least one user 2331a, 2331b, coupled wirelessly and accessing one or more software modules of the system including at least one enterprise application 2338 via an input and output (“I/O”) 2337c. In some embodiments, the computer system 2310 is configured to enable at least one user 2331a, 2331b, to be coupled to access enterprise applications 2338 via an I/O 2337c through LAN 2339a. In some embodiments, the user 2331 includes a user 2331a coupled to the computer system 2310 using a desktop computer, and/or laptop computers, or any fixed, generally non-mobile internet appliances coupled through the internet 2339b. In some embodiments, the user includes a mobile user 2331b coupled to the computer system 2310. In some embodiments, the user 2331b connects using any mobile computing 2331c to wireless coupled to the computer system 2310, including, but not limited to, one or more personal digital assistants, at least one cellular phone, at least one mobile phone, at least one smart phone, at least one pager, at least one digital tablets, and/or at least one fixed or mobile internet appliances.

The subject matter described herein are directed to technological improvements to the field of heart assist pumps by actuating one or more inflatable elements to move fluid. The disclosure describes the specifics of how a machine including one or more computers comprising one or more processors and one or more non-transitory computer readable media implement the system and its improvements over the prior art. The instructions executed by the machine cannot be performed in the human mind or derived by a human using a pen and paper but require the machine to convert process input data to useful output data. Moreover, the claims presented herein do not attempt to tic-up a judicial exception with known conventional steps implemented by a general-purpose computer; nor do they attempt to tic-up a judicial exception by simply linking it to a technological field. Indeed, the systems and methods described herein were unknown and/or not present in the public domain at the time of filing, and they provide technologic improvements advantages not known in the prior art. Furthermore, the system includes unconventional steps that confine the claim to a useful application.

It is understood that the system is not limited in its application to the details of construction and the arrangement of components set forth in the previous description or illustrated in the drawings. The system and methods disclosed herein fall within the scope of numerous embodiments. The previous discussion is presented to enable a person skilled in the art to make and use embodiments of the system. Any portion of the structures and/or principles included in some embodiments can be applied to any and/or all embodiments: it is understood that features from some embodiments presented herein are combinable with other features according to some other embodiments. Thus, some embodiments of the system are not intended to be limited to what is illustrated but are to be accorded the widest scope consistent with all principles and features disclosed herein.

Some embodiments of the system are presented with specific values and/or setpoints. These values and setpoints are not intended to be limiting and are merely examples of a higher configuration versus a lower configuration and are intended as an aid for those of ordinary skill to make and use the system.

Any text in the drawings is part of the system's disclosure and is understood to be readily incorporable into any description of the metes and bounds of the system. Any functional language in the drawings is a reference to the system being configured to perform the recited function, and structures shown or described in the drawings are to be considered as the system comprising the structures recited therein. Any figure depicting a content for display on a graphical user interface is a disclosure of the system configured to generate the graphical user interface and configured to display the contents of the graphical user interface. It is understood that defining the metes and bounds of the system using a description of images in the drawing does not need a corresponding text description in the written specification to fall with the scope of the disclosure.

Furthermore, acting as Applicant's own lexicographer, Applicant imparts the explicit meaning and/or disavow of claim scope to the following terms:

Applicant defines any use of “and/or” such as, for example, “A and/or B,” or “at least one of A and/or B” to mean element A alone, element B alone, or elements A and B together. In addition, a recitation of “at least one of A, B, and C,” a recitation of “at least one of A, B, or C,” or a recitation of “at least one of A, B, or C or any combination thereof” are each defined to mean element A alone, element B alone, element C alone, or any combination of elements A, B and C, such as AB, AC, BC, or ABC, for example.

“Substantially” and “approximately” when used in conjunction with a value encompass a difference of 5% or less of the same unit and/or scale of that being measured.

“Simultaneously” as used herein includes lag and/or latency times associated with a conventional and/or proprietary computer, such as processors and/or networks described herein attempting to process multiple types of data at the same time. “Simultaneously” also includes the time it takes for digital signals to transfer from one physical location to another, be it over a wireless and/or wired network, and/or within processor circuitry.

As used herein, “can” or “may” or derivations there of (e.g., the system display can show X) are used for descriptive purposes only and is understood to be synonymous and/or interchangeable with “configured to” (e.g., the computer is configured to execute instructions X) when defining the metes and bounds of the system. The phrase “configured to” also denotes the step of configuring a structure or computer to execute a function in some embodiments.

In addition, the term “configured to” means that the limitations recited in the specification and/or the claims must be arranged in such a way to perform the recited function: “configured to” excludes structures in the art that are “capable of” being modified to perform the recited function but the disclosures associated with the art have no explicit teachings to do so. For example, a recitation of a “pump configured to receive a fluid from structure X at an upper portion and deliver fluid from a lower portion to structure Y” is limited to systems where structure X, structure Y, and the pump are all disclosed as arranged to perform the recited function. The recitation “configured to” excludes elements that may be “capable of” performing the recited function simply by virtue of their construction but associated disclosures (or lack thereof) provide no teachings to make such a modification to meet the functional limitations between all structures recited. Another example is “a computer system configured to or programmed to execute a series of instructions X, Y, and Z.” In this example, the instructions must be present on a non-transitory computer readable medium such that the computer system is “configured to” and/or “programmed to” execute the recited instructions: “configure to” and/or “programmed to” excludes art teaching computer systems with non-transitory computer readable media merely “capable of” having the recited instructions stored thereon but have no teachings of the instructions X, Y, and Z programmed and stored thereon. The recitation “configured to” can also be interpreted as synonymous with operatively connected when used in conjunction with physical structures.

It is understood that the phraseology and terminology used herein is for description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The previous detailed description is to be read with reference to the figures, in which like elements in different figures have like reference numerals. The figures, which are not necessarily to scale, depict some embodiments and are not intended to limit the scope of embodiments of the system.

Any of the operations described herein that form part of the invention are useful machine operations. The invention also relates to a device or an apparatus for performing these operations. All flowcharts presented herein represent computer implemented steps and/or are visual representations of algorithms implemented by the system. The apparatus can be specially constructed for the required purpose, such as a special purpose computer. When defined as a special purpose computer, the computer can also perform other processing, program execution or routines that are not part of the special purpose, while still being capable of operating for the special purpose. Alternatively, the operations can be processed by a general-purpose computer selectively activated or configured by one or more computer programs stored in the computer memory, cache, or obtained over a network. When data is obtained over a network the data can be processed by other computers on the network, e.g., a cloud of computing resources.

The embodiments of the invention can also be defined as a machine that transforms data from one state to another state. The data can represent an article, that can be represented as an electronic signal and electronically manipulate data. The transformed data can, in some cases, be visually depicted on a display, representing the physical object that results from the transformation of data. The transformed data can be saved to storage generally, or in particular formats that enable the construction or depiction of a physical and tangible object. In some embodiments, the manipulation can be performed by a processor. In such an example, the processor thus transforms the data from one thing to another. Still further, some embodiments include methods can be processed by one or more machines or processors that can be connected over a network. Each machine can transform data from one state or thing to another, and can also process data, save data to storage, transmit data over a network, display the result, or communicate the result to another machine. Computer-readable storage media, as used herein, refers to physical or tangible storage (as opposed to signals) and includes without limitation volatile and non-volatile, removable and non-removable storage media implemented in any method or technology for the tangible storage of information such as computer-readable instructions, data structures, program modules or other data.

Although method operations are presented in a specific order according to some embodiments, the execution of those steps do not necessarily occur in the order listed unless explicitly specified. Also, other housekeeping operations can be performed in between operations, operations can be adjusted so that they occur at slightly different times, and/or operations can be distributed in a system which allows the occurrence of the processing operations at various intervals associated with the processing, as long as the processing of the overlay operations are performed in the desired way and result in the desired system output.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein. Various features and advantages of the invention are set forth in the following claims.

We claim:

1. A pump comprising:
a stent,
a pumping chamber,
one or more pump elements,
a first check element, and
wherein the pumping chamber is located within the stent;
wherein the pumping chamber comprises a chamber first end and a chamber second end;
wherein the first check element is configured to prevent backflow through the chamber first end of the pumping chamber; and
wherein the one or more pump elements are configured to simultaneously force fluid through the chamber first end and the chamber second end.

2. The pump of claim 1,
wherein the pump is configured to be placed in a descending aorta of a heart.

3. The pump of claim 2,
wherein the first check element is configured to permit flow out of the chamber first end when the one or more pump elements are inflated.

4. The pump of claim 3,
further comprising a second check element.

5. The pump of claim 4,
wherein the second check element is configured to restrict flow out of the chamber second end when the one or more pump elements are inflated.

6. The pump of claim 2,
further comprising a controller;
wherein the controller is configured to cause the one or more pump elements to inflate when an aortic valve is closed.

7. The pump of claim 1,
wherein the pump includes one or more inflatable stent shields configured to contact a wall of an aorta.

8. The pump of claim 7,
wherein the one or more inflatable stent shields include one or more flow channels configured to allow bypass flow between the stent and an aortic wall.

9. The pump of claim 7,
wherein the one or more inflatable stent shields comprise a plurality of discrete inflatable segments spaced to permit perfusion around the one or more inflatable stent shields.

10. The pump of claim 4,
wherein the second check element includes one or more bypasses.

11. The pump of claim 10,
wherein the one or more bypasses are configured to enable enough fluid to escape to pressurize an aortic arch greater than normal blood pressure when the one or more pump elements are inflated.

12. The pump of claim 4,
wherein the stent includes a first tapered region configured to house the first check element.

13. The pump of claim 12,
wherein the stent includes a second tapered region configured to house the second check element.

14. The pump of claim 4,
wherein the second check element comprises an inflatable balloon element configured to partially or fully restrict flow through the chamber second end.

15. The pump of claim 14,
wherein the inflatable balloon element is configured to allow regulated bypass flow to an upper extremities during pump inflation.

16. The pump of claim 1,
wherein the stent, the one or more pump elements, and the first check element are configured to be delivered to a deployment sight using a catheter having a diameter of 10 to 12 French.

17. The pump of claim 1,
wherein the one or more pump elements include a first pump element and a second pump element.

18. The pump of claim 17,
where in the first pump element is configured to direct fluid through the chamber first end.

19. The pump of claim 18,
where in the second pump element is configured to direct fluid through the chamber second end.

20. The pump of claim 19,
further including an inflatable chamber separator between the first pump element and the second pump element.
